SEQUENCE LISTING (45 sequences)

SEQ ID NO: 1 - DNA, 1410 bp, synthetic construct
  source 1..1410: mol_type = other DNA
atgccttcttggaaaaccctgttcctcctcctgggccctatcgccaccgctgccgctgctcccgttgacaagcagtctctgcctaccggcaacagcaccattagctcttgggtcagcaagcaggaggacatcagcttcagcgagatgctccgaaacgtcaaccctgagggcaccgccaagggcttcgttgccgcgtcgctcagcaccgctggccccgactacttctacacctggacccgagacgccgctctcgtttcccgagttattgcttacaagtacaacaccaccaacgctggcgactctaagatccacggcgttctggacgactacgttaacttccagattaacacccagtctgagcccaccccttgcaactgcctgggcgagcctaagttcaaccctgacggcagctctttcaccggcccttggggccgcccccagaacgacggccccgctgagcgagcctccagtttcatgctgattgccgactctttcctcagccagaccaagaacgcctcttacttcaccaacaccctcaagcctgccatttacaaggacctcgactacgtcgttgacacctggtctaacccttgcttcgacctgtgggaggaggttaacggcatccacttctacaccctcatggttatgcgacgatctctcctcgacggcgctaacttcgccacccgaaacggcgacaactccaaggcctcaacctactccggcgtcgccgccaagattcaggctcgactgaactctttctgggacgccggcaagaactacattaccgttacccaggactacaagaacggcgttgagaagcctagcggcctcgacgttagcaccctcattgctgctaacgtcgctggcatgggcgacggcttctacacccccggcagcgagcgcatcctcgctaccgctctggctttcgagaagagcatggcaagtctgtaccctctgaacaacaacctgcctagccacctgggcaacgccattggccgataccccgaggacacctacaacggcaacggcaactcccagggcaacccttggttcctcgctaccaccgctttcaccgagctgtactaccgcgccattctggagtggaagaacaccggcgttaccgtcacccctatttctaaggacttcttcgtccgattcgacagcagcgctgctcctggcaagaagtacaaccccggctcccaggagttcgctaccctgacccagtctattgctgccgccgctgaccgattcatgtccaccgtccagtaccaccagaaccctaacggctctctgtctgaggagttcgaccgatctagcggctacatgaccggcgcccgagacctgacctggagtcacgctgctttcattaccgctgctcaggcccgagctggctctccttctttctaataa

SEQ ID NO: 2 - protein, 468 aa, synthetic construct
MPSWKTLFLLLGPIATAAAAPVDKQSLPTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 3 - protein, 449 aa, synthetic construct
APVDKQSLPTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 4 - protein, 449 aa, Saksenaea vasiformis
APVDKQSLPTGNSTISSWVSKQEDISFSEMLRNVNPEGTAKGFVAASLSTAGPDYFYTWTRDAALVSRVIAYKYNTTNAGDSKIHGVLDDYVNFQINTQSESTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 5 - protein, 448 aa, synthetic construct
PVDKQSLPTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 6 - protein, 446 aa, synthetic construct
DKQSLPTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 7 - protein, 609 aa, Mucor ambiguus
MKFSSLLKKPLLLIAGILAATVVAETVPTTAEVKVKSFTYDGSTFAGQIYIKNIAYTKTVTVIYSDGSNNWNNNGNTIAASYSAAISGSNYEYWTFSAPVSSIKQFYVKYVVSGTTYYDNNSSGNYQVSTTTTTTTAPTTTTSGGSSTTTGGSTTTATSVPTAVPSGFPTGNSTISSWIDGQTSVSRYAMLRNINPAGTVTGFIAASMSTSGPDYFYAWTRDSALTSHVVAYDYNTTLAGNSTILGLLKNYVTFSLNSQTTSTVCNCLGEPKFNKDGSSYTGAWGRPQNDGPASRADTFILIADSILKQTGDATYVTGTLAPAIYKDLDYVVSTWSNGCFDLWEEVNGVHFYTLMVMRRGLVKGASFASRNGDSTRANTYTNTAASIKTKIDSFWNSNGQYVSVSQSVTGGVSKAGYDASVLIASNLGSLQDGFYTPGSDKMLATAVAIESKFASLYSINQNLNGYLGNAIGRYPEDTYNGNGNSQGNPWFICTNAFAELYYRAIKEWFNNGGVTVTSISLNFFKKFDSSAAVGTKYTVGTSAFNSLVQNVAVAADAFFSTIKFHAATNGSMSEQYGRTDGLMTGARDLTWSHASLISASYAKAGSPAA

SEQ ID NO: 8 - protein, 605 aa, Rhizopus microsporus
MKLMNPSIKACVFFILSYLSLLVSSAAVPTSAAVQVESYKYDGTTFSGRIFVKNIAYSKVVTVIYSDGSDNWNNNNNKISAAYSEAISGSNYEYWTFSAKVSGIKQFYVKYEVSGSTYYDNNGTKNYQVQATSATSTTATATTTTTTSTPATSTGPANNAPVSFPTGNSTISSWIKKQEEISRFAMLRNINPPGSATGFIAASLSTAGPDYYYSWTRDSALTANVIAYEYNTTFTGNTTLLKYLKDYVTFSVKSQSVSTVCNCLGEPKFNADGSSYTGPWGRPQNDGPAERAVTFMLIADSYLTQTKDASYVTGTLKPAIFKDLDYVVSVWSNGCYDLWEEVNGVHFYTLMVMRKGLILGADFAARNGDSSRASTYKKTASTMESKISSFWSDSNNYIQVSQSVTAGVSKKGLDVSTLLAANIGSLPDGFFTPGSEKILATAVALENAFASLYPINSNLPSYLGNSIGRYPEDTYNGNGNSQGNPWFLAVNAYAELYYRAISEWISNGKVTVSNISLPFFKKFDPSATSGKTYTAGTSDFDNLAQNIALGADRFLSTVKFHAYTNGSLSEEYDRSTGMSTGARDLTWSHASLITAAYAKAGSPAA

SEQ ID NO: 9 - protein, 605 aa, Rhizopus azygosporus
MKLMNPSMKACVFFILSYFSLLVSSAAVPTSAAVQVESYNYDGTTFSGRIFVKNIAYSKVVTVIHSDGSDNWNNNNNKVSAAYSEAISGSNYEYWTFSAKLSGIKQFYVKYEVSGSTYYDNNGTKNYQVQATSATSTTATATTTTATGTTTTSTGPTSTASVSFPTGNSTISSWIKNQEEISRFAMLRNINPPGSATGFIAASLSTAGPDYYYSWTRDSALTANVIAYEYNTTFTGNTTLLKYLKDYVTFSVKSQSVSTVCNCLGEPKFNADGSSFTGPWGRPQNDGPAERAVTFMLIADSYLTQTKDASYVTGTLKPAIFKDLDYVVSVWSNGCYDLWEEVNGVHFYTLMVMRKGLILGADFAARNGDSSRASTYKKTASTMESKISSFWSDSNNYIQVSQSVTAGVSKKGLDVSTLLAANIGSLPDGFFTPGSEKILATAVALENAFASLYPINSNLPSYLGNSIGRYPEDTYNGNGNSQGNPWFLAVNAYAELYYRAIKEWISNGKVTVSNISLPFFKKFDSSATSGKTYTAGTSDFNNLAQNIALGADRFLSTVKFHAYTNGSLSEEYDRSTGMSTGARDLTWSHASLITAAYAKAGSPAA

SEQ ID NO: 10 - protein, 582 aa, Backusella circina
DTVPTTQVKLQSYTYSGGVLSGTIYVQNIDYTKVVTVIYSDGSGNWNNNGNTISASYSSSISGTNYEYWTFSASVSSIQQFYISYLVDGTTYYDNNGGYGANYAVTATTTTASSTTKTTSGTTKTSTTSTATATSTGTSSFPSGNSTISTWSPSQHSISLYAMLRNINPPGSAAGFISASLSTSGPDYYYSWTRDSALVAHVIVNEYNTTYQGNSTLLGILKDYVTYSLNAQTTSTVCNCLGEPKFNPDGSSFTGAWGRPQNDGPAERAVSFIYFADSYLTQTSDSSYVTGTLAPAIYKDLDYVVSVWSNGCFDLWEEVNGIHFYTLMFMRRGLLDGANFASRNGDSTRASTYTSTAASIKTKIDGFWVSSGNYVQVSQSVTSGVSKAGYDASTLIAANQASRGDGFYTPGSDKMLATAVAIESQFSSLYSINTNKASYLGNAIGRYPEDTYNGNGNSQGNPWFLCTNAFGELFYRAISEWNAAGSVTVNSVNLAFFKKYDSSTSSGTTYTVGTSAYNNLVQNVALAADAYFSTVKYHALTNGSMSEQYDRSSGMATGARDLTWSHAAMITALKAKSGTPVY

SEQ ID NO: 11 - protein, 578 aa, Backusella circina
DTVPTTQVEVKTYTYSGGELSGTIYVENIDYTKVVTVIYADGSDDWNNNGNTIAAAFSESISDTNYEYWTFSSSVNSIKEFYVKYDVDGNTYYDNNGNANYQVSASSTTTTTATKTTTTATKTTATATSTATGSTSFPSGNSTISSWIKGQEDTSRSVMLGNINPPGSATGFISASLSTSGPDYYYHWTRDAALVAHVIVNDYNTTLSGDSSTLQVIKDYVTFSVNSQSESTVCNCLGEPKFNPDGSSYTGAWGRPQNDGPAERATTFIFFADTYLAQGGDSSYVTGTLAPAIYADLDYVVNNWSTGCYDLWEEVNGIHFYTLMVMRRGLIDGASFASRNGDSTRSSSYTSTAKSIATKIDSFWSASNNYVAVSQSVTSGVSKAGYDASTIIAANQASLGDGFYTPGSDKMLATSVAVENAFSSLYTVNTGKASYLGNAIGRYPEDTYNGNGNSQGNPWYLCTNALGELYYRAIKEWNAAGSVTVNSVNLGFFQKIDSSISSGTTFTLGTSDYSTLVDNVALAADKFFAVVQYHERSNGSMPEQFGREDGLPTGARDLTWSHAAMISAARAKAGTPVY

SEQ ID NO: 12 - protein, 577 aa, Benjaminiella poitrasii
ETVPTTSQVKLKSYTYDGSTLSGQIYIQNIAYTKVVTVIYSDASSNWNNNGNTIAASYTNGISGTNYEYWNFTGPVSSINAFYIKYDVSGNTYYDNNNNANYQVTKTSTTTTTSTATATTTTKTSTPTSTGVPSNFPTGNSTISTWLKSQIGISRYAMLRNINPAGSAVGFIAASLSTANPDYYYAWTRDSALTSYVIANDYNSTLAGNSTILQIMKDYVTFSVKSQSVSTACNCLGEPKFNPDGSSYTGAWGRPQNDGPAERATTFILFADSYLKQTGDASYVTGTLKPAIFKDLDYVVNTWTNGCFDLWEEVNGVHFYTLMVMRKGLIRGANFATRNGDSTRASTYTNTAASIKTKMDSFWSSGNNYIAVSQSVTGGVSKAGYDVANIIAANVGSLQDGVYTPGSDRILATAVAVEAKFASLYGVNSNLPGYLGNSIGRYPEDTYNGNGNSQGNPWFLATNAYAELYYRAIREWYDNGGVTVNSVNLPFFKKFDSSAASGTTYTVGTTAFNTMVSNVAAAADKFFSTVKFHAYTNGSMSEQFGRNDGLCTGARDLTWSHASLISAALAKAGTPSV

SEQ ID NO: 13 - protein, 574 aa, Choanephora cucurbitarum
QSVPTSDPVKVKTFSYDGNSFSGQIYIKNIAYEKTVTVIYSDGTGNWNNNNNKIAAAFSEAISGSNYEYWTFSASVPSIKQFYVKYDVSGKTYYDNNGSKDYNVVTSGPTTTSSGPSKTTTNGPAPTSTNTNFPSGNPSITSWIDKQIDISRSAMLKNINPAGTVKGFIAASLSTSNPDYFYAWTRDAALVAHVVANDYNRTKSGDATYLGLLKDYVTFSINSQNTPTACNCLGEPKFNKDGSGYNGPWGRPQNDGPAERADTFVLIADSILTQTKDVSYVTGTLRPAIYTDLDYVVRTWSNGCFDLWEEVNGVHFYTLMVMRRALLVGANFASRNGDSARASNYNNAANSIKSKIDSFWSSNNNYVAVSQSVTGGVSKAGYDVSTLIAANVGSLSDGFYTPGSERMLATAVAIEDKFANLYGINRNLDSSLGNAIGRYPEDTYNGNGNSQGNPWFIATNAYAELYYRAIKEWNNNGGVTVTNVNLNFFKKFDGSASVGTKYTAGSAAYNTLTQNIALAADKFFNTVKVHAATNGSMSEQYHRDTGSMTGARDLTWSHASLITAALAKAGTPVA

SEQ ID NO: 14 - protein, 584 aa, Rhizopus stolonifer
ASIPSSSEVQLDTYAYDGAKFSGKIYVKNIAYEKTVTVVYADGSDNWNNNGNIISATFSTPISGSNYEYWTFSSSISGIKEFYIKYVVSGKTYYDNNGTKNYQVSTTSSTTTASTTTATRTTTAGTTSTSTTAAPTSTSSGSFPSGNSTVSSWIKRQEKISRFAMLRNINPPGSAAGFIAASLSTSGPDYYYSWTRDSALTSNLIAYEYNTTLSGNTTILNILKDYVKFSISSQTTSTVCNCLGEPKFNHDGSSYTGAWGRPQNDGPAERANTFILFADSYLDQTKDASYVTGTLKPAIFKDLDYVVNVWSNGCYDLWEEVNGVHFYTLMVMRKGLLLGADFAKRNGDSTRASTYTNTASTIATKISSFWVSSSNWIQVSQSVTAGVSKKGLDVSTLLAANLGSVEDGFFTPGSEKILATAVAIEDAFASLYPINSNLPSYLGNSIGRYPEDTYNGNGNSQGNPWFLAVNGFAELYYRAIKEWVNNGSVTVSNISLSFFKKFDSSATAGKTYTAGTADFNNLAQNIALGADRFLSTVQTHAFNNGSLAEEYDRTTGVSTGARDLTWSHASLITAAYAKAGSPAA

SEQ ID NO: 15 - protein, 584 aa, Mucor circinelloides
ETVPTTAAVKVKSFTYDGSTFAGQIYVKNIAYTKTVTVIYSDASNNWNNNGNTIAASYSEGISGTNYEYWTFSAPVSGIKQFYVKYVVSGTTYYDNNNSGNYQVTTTTTTTAPTSTTSGGSSTTTGGSTTTATSVPTGVPSGFPTGNSSISSWIDGQTSVSRYAMLRNINPAGAVSGFIAASMSTSGPDYFYAWTRDSALTSHVVAYDYNTTLAGNSTILGLLKNYVTFSINSQTTSTVCNCLGEPKFNKDGSSYTGAWGRPQNDGPASRADTFILIADSILKQTGDATYVTGTLAPAIYKDLDYVVSTWSNGCFDLWEEVNGVHFYTLMVMRRGLIKGANFASRNGDNTRANTYTNTAASIKTKIDSFWNSNGNYVTVSQSVTGGVSKAGYDASVLIAANLGSVQDGFYTPGSDKMLATAVAIESKFASLYSINQNLNGYLGNAIGRYPEDTYNGNGNSQGNPWFICTNAFAELYYRAIKEWFNNGGVTVTSISLNFFKKFDSSAAVGTKYTVGTSAFNSLVQNVAVAADAFFSTVKFHAATNGSMSEQYGRSDGLMTGARDLTWSHASLISASYAKAGSPAA

SEQ ID NO: 16 - protein, 581 aa, Dichotomocladium elegans
ATVPKTQVKLEAYTYSDSIFSGRIFVQNIDYTKIVTVYWSDASNKWDSSKYYTEAAYTHSIPGTNYEYWDFSATIGPSGIKQFYISYQVRGVTYYDNNGGYGVNYDVISSPPTTSDATTIPTTTTSAIPSSTTTIPASTGVPSGNSTITVWADSQHKISWKAMLANINPPGSATGFIAASLSTSGPDYYYAWTRDAAMVAHVIVNAYNTTKAGDATTLGVLKDFVTFQIKAMATSTVCNCLGEPKFNPDGSSYTGPWGRPQNDGPAERATTFILFADSYLSQTGDAAYVTTLKRAIFTDLDYVVTTWQDNCFDLWEEVNGLHMYTLAVMRRSLVDGASFAARNGDNTRASTYTNTAKSIETKLASFYNSSSNYVVVTKNFAGGVNKAGLDTSTLIAANTAGLGDGFFTPGSPEILATAAAIEKSFADLYGINKQIPNWLGTAIGRYPEDTYNGNGNSQGNPWFICTATFAELYYRAILEWQHAGSVTVNNVNLPFFKKFDSSTSSGTTYTVGTSSFDSLISKVAYAADNFFSTIKYHAATNGSMSEQFNRDTGFMTGARDLTWSHAAFITAAKAKAGTPVY

SEQ ID NO: 17 - protein, 582 aa, Fennellomyces sp.
QTVPSVPIKLESYTYSDNVFAGRIFIRNIAYTKVVKVFWSDESGNWNGNGNYVDASYSGSISGTNYEYWEFSTTIGSAGISQSYLRYDVSGSTYYDNNGNANYDIEEDSTPTPTTTTTSTTTITTTTTSGTSTPTSVPTEVPAGNTTVTDWVNDQLEISWPSLLKNVNPSGAVTGFIAASLSTNDPDYFYCWTRDAALVARVMVYMYNTTEAGDTSLRSKLQDYVTFQINSMKTATVCNCLGEPKFNKDGSGYSGAWGRPQNDGPADRAITLILFADSFIAQGGDVSYITNTLKPAIYTNLDYVVNTWSNVCFDLWEEVNGVHIYTLSVMRKGLLEGADFASRNGDSTRANTYRSTASSIKTRLESFWSSSNNYITVTQSYSGGVNKAGLDVSTLLAANGASMNDGFYTPGSDKMLATAVAIENSFAGIYAVNQNRPDWKGTAIGRYPEDTYDGHGNSQGNPWFIATAAYAEMYYRAILEWQQQPSITVNSINLSFFKKFDSSAAVGTVYKPGTQAFNNMVSNVAFAADEFFSTMNFHSATNGSMSEQYNRNTGIMQGARDLTWSHAAFITAAKAKLGTPVF

SEQ ID NO: 18 - protein, 577 aa, Gilbertella persicaria
ETVPTSAQVQVKSFNYDGSTLSGQIYVQNIAYEKTVTVVYSDGSDNWNNNGNTIAASYSSSISGSNYEYWTFSSSVPSIKQFYIKYVVAGKTYYDNNGTKNYQVSASTPTTTTTTTSSGATKTTTTIGPTSTSTVFPSGNSTISSWLKGQIETSRFAMLRNINPAGTVKGFIAASLSTANPDYFYAWTRDAALVGHVIANDYNRTLAGNSTYLGLLKDYVTFSVNSQSTSTICNCLGEPKFNKDGSGYSGAWGRPQNDGPAERADTFILIADSILKQTGDATYVTGTLRPAIYKDLDYVVNVWSNGCFDLWEEVNGVHFYTLMVMRRSLILGANFASRNGDSTRASTYTNTANSIKTKIDTFWSSSNNYVAVSQSVTGGVNKAGYDVANLIAANVGSLDDGFYTPGSERILATAVAVESKFASIYGVNQNLPSWLGNSIGRYPEDTYNGNGNSQGNPWFIATNTYAELYYRAIKEWTNNGGVTVTNVNFNFFKKFDSSASVGTKYTVGTSAFNTLTQNVALAADNFFSTVKVHAATNGSMSEQFGRDSGVMTGARDLTWSHASLITAALAKTGAPVA

SEQ ID NO: 19 - protein, 585 aa, Mucor circinelloides
ETVPTTEAVKVKSFTYDGSTLAGQIYIKNIAYTKTVTVIYSDASDNWNNNGNTIAASYSAAIAGTNYEYWTFSAPVSGIKQFYVKYVVSGTTYYDNNNSGNYQVSVTTTTTTAPTTTTSGGSSTTTGGSTTTATSVPTGVPSGFPTGNSTISSWIDGQTSVSRYAMLRNINPAGAVTGFIAASMSTSGPDYFYAWTRDSALTSHVVAYDYNTTLAGNSTILGLLKNYVTFSLNSQTTSTVCNCLGEPKFNKDGSGYSGAWGRPQNDGPASRADTFILIADSILKQTGDATYVTGTLAPAIYKDLDYVVSTWSNGCFDLWEEVNGVHFYTLMVMRRGLVKGANFASRNGDSTRATTYTNTAASIKTKIDSFWNSNGQYVSVSQSVTGGVSKAGYDASVLIASNLGSLQDGFYTPGSDKMLATAVAIESKFASLYSINQNLNGYLGNAIGRYPEDTYNGNGNSQGNPWFICTNAFAELYYRAIKEWFNNGGVTVTSISLNFFKKFDSSAAVGTKYTVGTSSFNSLVQNVAVAADAFFSTIKFHAATNGSMSEQYGRTDGLMTGARDLTWSHASLISASYAKAGSPAA

SEQ ID NO: 20 - protein, 583 aa, Circinella umbellata
APTSEIELDSYTYTGGVFSGRLYVKNIAYTKEVNVYWSDASEDWANNGNYVAATYSEAISGTNYEYWEFSAKIGSSGISESYIKYTVSGSTYYDNNGGKNYAITETTTPTITATPTSTTATTTISSTTTTATSIPTSVPSTVPEGNVTVTEWVNKQLKISWSDLLQNVNPSGTVTGFIAASLSTSNPDYFYCWTRDAAMVARVMTYMYNTTEAGDSSLESALKDYITFQINSMKTATACNCLGEPKFNTDGSGYSGPWGRPQNDGPAERATTMILFADSYLAQGGDTSYVTNTLKPAIYTNLDYVVGTWSNNCFDLWEEVNGVHIFTLAVMRKSLLDGADFAARNGDTSRVSGYQSTASSIKTKLESFWSSSNNYITVTQSYSGGVQKAGLDVSTLIAANQGSMGDGFYTPGSDKMLATAVAIENSFANIYTINKNKPSWLGTAIGRYPEDTYNGNGNGQGNPWFIATATYAELYYRAILEWQQQESVTVNSVNFDFFSKFDSSAKVGTVYTPGTDTFNTMVSNVAFAADEFLSTMEHYAATNGSMSEQFNRETGSLTGARDLTWSHAAFITAGKAKLGIPSF

SEQ ID NO: 21 - protein, 585 aa, Mucor cordense
ETVPTTANVLVKSYTWDGATLSGQIYIKNLAYAKVVSVIYSDANDNWNNNGNKVAASYSAGIDGTNYEYWTFSGAVSGIKQFYVKYDVSGTSYYDNNGTKNYQVTKTSTTTTAATTTTTATTTTGGTGTTTTTTATATATSTDFPTGNSTITTWLKSQEDISRGAMLRNINPPGAATGFIAASLSTSGPDYYYAWTRDSALTSHVIAHDYNTTLAGNTTILNILKDYVTFSVKSQSVSTVCNCLGEPKFNPDGSSYTGAWGRPQNDGPAERASTFILFGDSYLKQTGDATYVTGTLAPAIYKDLDYVVNTWSNGCFDLWEEVNGVHFYTLMSMRRGLLDGANFAKRNGDTTRATTYTNTAASIATKIDTFWVSSGNYVQVSQSVTGGVSKAGYDCANILAANMAANHDGFYTPGSSKILATAVAIESKFASLYSINSGLASWLGTAIGRYPEDTYNGNGNSQGNPWFICTNAFAELYYRAIKEWTQAGSVTVDSTSLNFFKKFDSSAAAGTKYTVGTSAFTNLVQNIANGADKFLSTSKFHAATNGSMSEQYNRDSGLMTGARDLTWSHASLISASRAKAGSPSL

SEQ ID NO: 22 - protein, 590 aa, Phascolomyces articulosus
DVPTSANIELDYYTYKDKVFSGRIYVKNIAYEKTVAVYWSDASGDWNNNGNNVAASYSESISGTDYEYWDFSTTIGSGGIKQSYLKYTVSGNTYYDNNGSNNYDITETTATTTTTTTTTTTTGSTATTSTTSGPTSTTSTTPGPTPTNVPDGNVTVTEWVNTQFAISWPTLLKNVNPSGTVKGFIAASLSTNNPDYFYSWTRDSALVAHTMTYLYNTSEAGDSTIESALKDYVTFSINAMNAATVCNCLGEPKFNTDGSGYTGAWGRPQNDGPASRATTMILFADSFLAQGGDVSYVINTLKPAIYKDLDYVVSTWSNTCYDLWEEVNGVHIYTLSVMRRALIDGANFAQRNGDTSRVSGYTSTATTIKTRLESFWSDSNNYITVTQSYSGGVQKAGLDVSTLIAANIGSVGDGFYTPGSDKVLATAVAIEKSFANLYTINQNKPSWLGNAIGRYPEDTYDGYGNSKGNPWFIATATYAELYYRAILEWQQQASVTVNSINLGFFSKFDSSASVGTVYTPGTDSFANMVSNVAFAADEFLSTIDYHAMNNGSMHEQYNRDTGISQGARDLTWSHAAFITAAKAKLGAPAF

SEQ ID NO: 23 - protein, 578 aa, Rhizopus microsporus
VPTSAAVQVESYKYDGTTFSGRIFVKNIAYSKVVTVIYSDGSDNWNNNNNKISAAYSEAISGSNYEYWTFSAKLSGIKQFYVKYEVSGSTYYDNNGTKNYQVQATSATSTTATATTTTSTSTTTTSTGPTSTASVSFPTGNSTISSWIKNQEEISRFAMLRNINPPGSATGFIAASLSTAGPDYYYSWTRDSALTANVIAYEYNTTFAGNTTLLKYLKDYVTFSVKSQSVSTVCNCLGEPKFNADGSSYTGPWGRPQNDGPAERAVTFMLIADSYLTQTKDASYVTGTLKPAIFKDLDYVVSVWSNGCYDLWEEVNGVHFYTLMVMRKGLILGADFAARNGDSSRASTYKNTASTMESKISSFWSDSNNYVQVSQSVTAGVSKKGLDVSTLLAANIGSLPDGFFTPGSEKILATAVALENAFASLYPINSNLPSYLGNSIGRYPEDTYNGNGNSQGNPWFLAVNAYAELYYRAIKEWISNGKVTVSNISLPFFKKFDSSATSGKTYTAGTSDFNNLAQNIALGADRFLSTVKFHAYNNGSLSEEYDRSTGMSTGARDLTWSHASLITAAYAKAGSPAA

SEQ ID NO: 24 - protein, 443 aa, Spinellus fusiger
VPSGNSTITAWGSKQDGISFSTMLGNINPPGSSKGFIAASLSTAGPNYYYSWTRDSALVARAITYKYSTSYQNDPKILGLLKDYVTYQVNEQTESTVCNCLGEPKFNPDGSSFSGPWGRPQNDGPAERASTMILFAKSYYAQTNDVGYVSNTLKPAIYKDLDYIVNVWGNNCFDLWEEVNGVHFYTLMMMRHGLVQGSIFANTLGDSTRANTYKTAAQNIKNRIDTFWESGSNYIVVTQNQSAGVNKPSGLDVAVLLAANQGGLGDGVYTPGSDKVLATAVALEKSFASLYPINKNLPSYYGTAIGRYPEDTYNGNGNSEANPWFIATTTYAELYYRAISEWTNGSGVTVNSINKEFFSKFDASATNGKVYTPGSDSFNSLVNNVAIAADNFLSTVRYHQTSNGSLSEQFNRYTGFMTGARDLTWSHAAMVTALAAKAGTPSP

SEQ ID NO: 25 - protein, 588 aa, Syncephalastrum racemosum
ATVPTTQVQLDYYTYSNNVLSGRIYVQNIAYSKVVKVIYSDASGNWNNNGNTISASYVESISGTNYEYWDFSATIGTAGIKQFYLRYDVSGSTYYDNNGGDNNNYNVVATTSTTSSSTTTTTATTTTATKTTSTTSATATPTSSATFPSGNSTITTWAKSQKDISWKTLLTSLNPSGTAKGFIAASLSTSNPDYYYAWTRDSALVARTMVNMYNTTEAGSASVLGLLQDYVTFQINAMSTSTVCNCLGEPKFNPDGSSYTGAWGRPQNDGPAERASTFILFADSYIAQGGQLSYVTGTLAPAIYKDLNYVVSTWSNNCFDLWEEVNGRHMFTLAVMRRALLDGVNFASRIGDTTYSSTWSSTASSIQSTLSGYYLSSGNYITTVQNFQSGVSKAGYDVSTLIAANVAGMGDGFFTPGSDEVLATAVAIENKFSSLYGVNANKASYLGTAIGRYPEDTYNGYGNGQGNPWFIATAAYAELYYRAILEWQTKSSIVVNSKNLGFFSKFDSSAAVGTTYTPGTTAYSNMVQNVALAADRFLSTVQLHAATNGSMSEQFNRDTGVMQGARDLTWSHSAFITAARAKLGSPVY

SEQ ID NO: 26 - protein, 448 aa, Syncephalastrum racemosum
ANSTVPDGNSTTTAWVKKQEAISWTDLKTNVNPEGAAKGFIAASLSTSEPDYYYAWTRDSALVARVMVNKYNTTDAGDANLLGLLQDYVSFQINAMGESTVCNCLGEPKFNPDGSSYTGAWGRPQNDGPAERASTFILLADSMIAQKSANGSYVSDTLAPAIYKDLAYVASTWENACYDLWEEVNGKHMYTLSVMRRALLDGADFASRQGQTANVTSWKSTADKIKSSLEGFFSSDNGYIEVTQDMQGGVQKKGLDVSTLIAANIGSMGDGFYTPGSDEVLATAVAVEAAFADLYKINQNTNSTGLGTAIGRYPEDTYNGVGNSQGNPWFIATNTFAELYYRAILEWQNKGTITVNSVNAAFFSKFDSSAKAGTTYKSGSTEFDSLINKVALAADAFLNTVQTYAASNGSMSEQYNRDTGALTGARDLTWSHASLITAANAKLGTPYN

SEQ ID NO: 27 - protein, 452 aa, Thermomucor HACC 243
VVTAAAAASVPSGNATITSWIQEQLAISWNTMLTSVNPVGAVTGFIAASLSTANPDYYYCWTRDAALVARVMTFMYNTTQAGDSGLLGILQDYVSFQIHAMGESTVCNCLGEPKFNPDGSSYTGAWGRPQNDGPAERASTFIKIADSYLTQTGDVSYVTNTLKPAIYEDLDYIVNVWQNTCFDLWEEVYGMHMYTLAVMRRGLLDGADFATRNGDTDKASTYTSTATSIQTRLATFWSDSNGYITVTQDYQSGVSKAGLDVSTLIAANVAGMNDGFFTPGSDEILATAVKVEAAFANLYGININKASYLGNAIGRYPEDTYNGNGNSEGNPWFIATAAFAELYYRAIMEWQLRGSSITVNSVNQGFFTKFDPSATAGTTYTPGTDAFNSLIDNVALAADQFFSTIHLHRATNGSMSEQYNRDTGFMQGARDLTWSHAAFITAAKAKQGTPSF

SEQ ID NO: 28 - protein, 589 aa, Zychaea mexicana
VDVPSVPIQLESYTYSENVFAGRIFVQNIAYTKEVNVFWSDASDDWNDNGNSVAASYSESIADTNYEYWEFSTTIGSAGISQSYLRYDVSGSIYYDNNDSQNYDITETSTPTTTTAAPTSTTSTPTTTTDGGSTTTTTATSVPTSTGVPEGNATISEWASAQLDISWPNLMMNVNPSGAVTGSIVASLSTSNPDYFYIWTRDAAMVARVMVYMYNTTEAGDTNLLNALTDYVTFSISSMNVDTVCDCLGEPKFNVDGSGYTGAWGRPQNDGPAERASTMILIADSYIAQGGDVSYVTDTLKPAIYTDLDYVVDTWSNVCFDLWEEVNGIHMYTLSVMRKALLDGANFATRNGDTSRVSGYESTASSIKTRLESFWSSSNNYITVTQSFSGGVSKAGLDVSTLIAANLASMNDGFYTPGSDEILATAVAIENSFISEYTLNQNRPSWLSTAIGRYPEDSYDGYGNSQGNPWFIATATYAELYYRAILEWQQQASISVNSVNLGFFSKFDSDASVGTVYTPGTEDFANMVSNVAFAADEFLATIENHSAVNGSLSEQYNRDTGIMQGARDLTWSHAAFITAAKAKQGAPIH

SEQ ID NO: 29 - protein, 449 aa, Saksenaea oblongispora
APVNKQSFPTGNPDISSWVSKQEGISFSEMLRNVNPPGTAKGFVAASLSTAGPDYFYTWTRDAALVSRVIAYKYNTTNAGDNNIHGALQDYVTFQINTQTESTPCNCLGEPKFNPDGSSFTGPWGRPQNDGPAERASSMILIADSFLAQTKDTAYVTNTLKPAIYKDLDYVVNTWSNPCFDLWEEVNGVHFYTLMVMRRGLLDGADFATRNGDASKASSYSDAASKIKTRLDSFWVSDKNYITVTQDYKEGVKKDTGLDVSTLIAANVAGKDDGFYTPGSDKILATAVAFENAMAKLYPLNQNLDSHLGNSIGRYPEDTYNGNGNSQGNPWFLATTAFTELYYRAILEWKDSGVTVTPVSKDFFAKFDSNAVAGKKYSPNSEEFATLTANIAAAADRFMSTVKYHQNPNGSLSEQFDRHSGYMTGARDLTWSHAAFITAAQARAGTPSF

SEQ ID NO: 30 - protein, 443 aa, Apophysomyces ossiformis
AIPTGNSSLNAWVSSQEDISFSVMLGNINPPGTVKGFVAASLSTAGPDYFYSWVRDSALVSRVVTHKYNTTETGNSTVAGFLEDYVHFQINTQTESTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSNKNYIIVTQDYQNGVQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSNEFASLAQNIASAADRFLSTVNYHRNSNGSLSEEFDRNTGYMTGARDLTWSHAAFITAGLARAGTPSF

SEQ ID NO: 31 - protein, 442 aa, Apophysomyces elegans
AIPTGNSSLNAWVSSQEDISFSVMLGNINPPGTVKGFVAASLSTAGPDYFYSWVRDSALVSRVVTHKYNTTETGNSTVAGFLQDYVHFQINTQTESTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIMVTQDYQNGIQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSDEFNSLAQNIASAADRFLSTVNYHRNSNGSLSEEYDRNSGYMTGARDLTWSHAAFITAGLARAGTPS

SEQ ID NO: 32 - protein, 443 aa, Apophysomyces variabilis
AIPTGNSSLNAWVSSQEDISFSVMLGNINPPGTVKGFVAASLSTAGPDYFYSWVRDSALVSRVVTHKYNTTETGNSTVAGFLQDYVHFQINTQTESTVCNCLGEPKFNPDGSSYTGPWGRPQNDGPAERASTMILIADSFLAQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIMVTQDYQNGVQKPSGLDISTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSDEFNSLAQNIASAADRFLSTVNYHRNSNGSLSEEYDRNSGYMTGARDLTWSHAAFITAGLARAGTPSF

SEQ ID NO: 33 - protein, 443 aa, Apophysomyces trapeziformis
AIPTGNSSLNAWVSSQEDISFSVMLGNINPPGTVKGFVAASLSTAGPDYFYSWVRDSALVSRVVTHKYNTTETGNSTVAGFLQDYVHFQINTQTESTVCNCLGEPKFNPDGSSYTGPWGRPQNDGPAERASTMILIADSFLTQTKNTSYVDNTLKPAIYKDLDYVVNTWSNPCFDLWEEVNGIHFYTLMVMRRGLLDGANFATRNGDTSKASSYSSTASQIQNKIDSFWSSSKNYIIVTQDYQNGVQKPSGLDVSTLIAANVAGMNDGFYTPGSDKMLATAVAIENAMANLYPLNKNLPSYLGNAIGRYPEDTYNGDGNSQGNPWFLATTAFSELYYRALLEWQETGVTVTSISKDFFSKFDPNASAGKKYSPGSNEYASLAQNIASAADRFLSTVNYHRNPNGSLSEEYDRNTGYMTGARDLTWSHAAFITAGLARAGTPSF

SEQ ID NO: 34 - protein, 449 aa, synthetic construct
APVDKQSLPTGNSTISSWVSKQEDISFSEMLRNVNPEGTAKGFVAASLSTAGPDYFYTWTRDAALASRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 35 - protein, 447 aa, synthetic construct
VDKQSLPTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 36 - protein, 444 aa, synthetic construct
QSLPTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 37 - protein, 443 aa, synthetic construct
SLPTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 38 - protein, 449 aa, synthetic construct
APVDKQSLPTGNSTISSWVSKQEDISFSEMLRNVNPEGTAKGFVAASLSTAGPDYFYTWTRDAALVSRVIAYKYNTTNAGNSTIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 39 - protein, 449 aa, synthetic construct
APVDKQSLPTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNTSYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 40 - protein, 449 aa, synthetic construct
APVDKQSLPTGNSTISSWVSKQEDISFSEMLRNVNPEGTAKGFVAASLSTAGPDYFYTWTRDAALVSRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSNASPGKKYNPGSQEFATLTQSIAAAADRFMSTVQYHQNPNGSLSEEFDRSSGYMTGARDLTWSHAAFITAAQARAGSPSF

SEQ ID NO: 41 - protein, 449 aa, synthetic construct
APVDKQSLPTGNSTISSWVSKQEDISFSEMLRNVNPEGTAKGFVAASLSTAGPDYFYTWTRDAALVSRVIAYKYNTTNAGNSTIHGVLDDYVNFQINTQSEPTPCNCLGEPKFNPDGSSFTGPWGRPQNDGPAERASSFMLIADSFLSQTKNTSYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 42 - protein, 449 aa, synthetic construct
APVDKQSLPTGNSTISSWVSKQEDISFSEMLRNVNPEGTAKGFVAASLSTAGPDYFYTWTRDAALASRVIAYKYNTTNAGNSTIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 43 - protein, 449 aa, synthetic construct
APVDKQSLPTGNSTISSWVSKQEDISFSEMLRNVNPEGTAKGFVAASLSTAGPDYFYTWTRDAALASRVIAYKYNTTNAGDSKIHGVLDDYVNFQINTQSEPTPCNCLGEPKFNPDGSSFTGPWGRPQNDGPAERASSFMLIADSFLSQTKNTSYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

SEQ ID NO: 44 - protein, 449 aa, synthetic construct
APVDKQSLPTGNSTISSWVSKQEDISFSEMLRNVNPEGTAKGFVAASLSTAGPDYFYTWTRDAALASRVIAYKYNTTNAGDSKIHGVLDDYVNFQINTQSEPTPCNCLGEPKFNPDGSSFTGPWGRPQNDGPAERASSFMLIADSFLSQTKNASYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSNASPGKKYNPGSQEFATLTQSIAAAADRFMSTVQYHQNPNGSLSEEFDRSSGYMTGARDLTWSHAAFITAAQARAGSPSF

SEQ ID NO: 45 - protein, 449 aa, synthetic construct
APVDKQSLPTGNSTISSWVSKQEDISFSEMLRNVNPEGTAKGFVAASLSTAGPDYFYTWTRDAALASRVIAYKYNTTNAGNSTIHGVLDDYVNFQINTQSEPTPCNCLGEPKFNPDGSSFTGPWGRPQNDGPAERASSFMLIADSFLSQTKNTSYFTNTLKPAIYKDLDYVVDTWSNPCFDLWEEVNGIHFYTLMVMRRSLLDGANFATRNGDNSKASTYSGVAAKIQARLNSFWDAGKNYITVTQDYKNGVEKPSGLDVSTLIAANVAGMGDGFYTPGSERILATALAFEKSMASLYPLNNNLPSHLGNAIGRYPEDTYNGNGNSQGNPWFLATTAFTELYYRAILEWKNTGVTVTPISKDFFVRFDSSAAPGKKYNPGSQEFATLTQSIAAAADRFMSTVQYHQNPNGSLSEEFDRSSGYMTGARDLTWSHAAFITAAQARAGSPSF

(19) United States
(12) Patent Application Publication
(10) Pub. No.: US 2025/0236858 A1
(43) Pub. Date: Jul. 24, 2025

(54) GLUCOAMYLASE VARIANTS AND METHODS FOR USE THEREOF

(71) Applicant: DANISCO US INC., PALO ALTO, CA (US)

(72) Inventors: Martijn SCHEFFERS, Warmond (NL); Svetlana Laura IANCU, Wageningen (NL); Marco VAN BRUSSEL-ZWIJNEN, LEMELE (NL); Paulien KRUITHOF, Zoetermeer (NL); Robin SORG, Leiden (NL); Zhongmei TANG, Shanghai (CN); Zhenghong ZHANG, Shanghai (CN); Yuepeng SHANG, Shanghai (CN); Barbara KOZAK, Utrecht (NL); Igor NIKOLAEV, Noordwijk (NL); Jonathan LASSILA, San Francisco, CA (US)

(21) Appl. No.: 18/701,413

(22) PCT Filed: Oct. 14, 2022
(86) PCT No.: PCT/US2022/078121

§ 371 Date: Apr. 15, 2024

(30) Foreign Application Priority Data
Oct. 15, 2021 (WO) PCT/CN2021/124148

(51) Int. Cl. C12N 9/34; A23K 20/189; C12G 3/02; C12N 9/26; C12N 9/44; C12N 15/52; C12N 15/75; C12N 15/81; C12P 7/06; C12P 7/16; C12P 19/14

(52) U.S. Cl. CPC C12N 9/2428; A23K 20/189; C12G 3/02; C12N 9/2414; C12N 9/2457; C12N 15/52; C12N 15/75; C12N 15/815; C12P 7/06; C12P 7/16; C12P 19/14; C12Y 302/01001; C12Y 302/01003; C12Y 302/01041

(57) ABSTRACT

Described herein, inter cilia, are glucoamylase variants and methods of using the same for saccharifying a starch substrate. Moreover, the disclosure also relates to a process of producing fermentation products and a method for increasing starch digestibility in an animal as well as a method of producing a fermented beverage using said as well as a. method of producing a fermented beverage using said glucoamylase variants.

### CROSS-REFERENCE TO RELATED APPLICATIONS

This application claims priority to International Patent Application No. PCT/CN2021/124148, filed Oct. 15, 2021, the disclosure of which is incorporated by reference herein in its entirety.

### REFERENCE TO AN ELECTRONIC SEQUENCE LISTING

The contents of the electronic sequence listing (NB42043-WO-PCT2_sequencelisting.xml; Size: 62,000 bytes; and Date of Creation: Oct. 11, 2022) is herein incorporated by reference in its entirety.

### FIELD OF THE INVENTION

The present disclosure relates compositions comprising glucoamylase variants and methods of saccharifying a starch substrate as well as methods for producing fermentation products using the same.

### BACKGROUND

Glucoamylase (1,4-alpha-D-glucan glucohydrolase, EC 3.2.1.3) is an enzyme which catalyzes the release of D-glucose from the non-reducing ends of starch or related oligo- and poly-saccharide molecules. Glucoamylases are produced by several filamentous fungi and yeast.

The major application of glucoamylase is the saccharification of partially processed starch/dextrin to glucose, which is an essential substrate for numerous fermentation processes. The glucose may then be converted directly or indirectly into a fermentation product using a fermenting organism. Examples of commercial fermentation products include alcohols (e.g., ethanol, methanol, butanol, 1,3-propanediol); organic acids (e.g., citric acid, acetic acid, itaconic acid, lactic acid, gluconic acid, gluconate, lactic acid, succinic acid, 2,5-diketo-D-gluconic acid); ketones (e.g., acetone); amino acids (e.g., glutamic acid); gases (e.g., H₂ and CO₂), and more complex compounds.

The end product may also be syrup. For instance, the end product may be glucose, but may also be converted, e.g., by glucose isomerase to fructose or a mixture composed almost equally of glucose and fructose. This mixture, or a mixture further enriched with fructose, is the most commonly used high fructose corn syrup (HFCS) commercialized throughout the world.

Glucoamylase for commercial purposes has traditionally been produced employing filamentous fungi, although a diverse group of microorganisms is reported to produce glucoamylase since they secrete large quantities of the enzyme extracellularly. However, commercially used fungal glucoamylases have certain limitations such as slow catalytic activity or lack of stability that increase process costs.

There continues to be a need for new glucoamylases and glucoamylase variants to improve the efficiency of saccharification and provide a high yield in fermentation products.

### SUMMARY

The present disclosure relates to a recombinant host cell, a composition comprising a glucoamylase and methods of saccharifying the starch substrate using the glucoamylase. Moreover, the disclosure also relates to a process of producing fermentation products and a method for increasing starch digestibility in an animal as well as a method of producing a fermented beverage.

Accordingly, in some aspects, provided herein is a glucoamylase variant or fragment thereof comprising one or more amino acid substitutions at residue positions corresponding to positions 20, 21, 23, 37, 49, 51, 52, 66, 67, 69, 73, 77, 79, 80, 81, 84, 92, 94, 102, 119, 121, 134, 140, 141, 143, 156, 157, 158, 164, 165, 166, 172, 192, 203, 210, 213, 214, 215, 218, 221, 222, 233, 235, 236, 238, 243, 252, 253, 274, 278, 281, 290, 302, 310, 321, 338, 341, 350, 351, 352, 354, 370, 390, 403, 404, 405, 416, 418, 422, 430, 434, 440, 441, 444, 445, and/or 449 of SEQ ID NO:4 and/or an equivalent position in a parent glucoamylase. In some embodiments, the equivalent position is determined by sequence identity and said parent glucoamylase has at least 80% sequence identity and less than 100% sequence identity with SEQ ID NOs:2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37. In some embodiments, the equivalent position is determined by sequence identity and wherein the parent glucoamylase comprises SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37. In some embodiments of any of the embodiments disclosed herein, the parent glucoamylase is a Mucorales-clade glucoamylase. In some embodiments of any of the embodiments disclosed herein, said one or more amino acid substitutions comprise one or more of X020A/E/F/G/P; X021M/S/T/W; X023L/M; X037C/N; X049W; X051K/L/V/Y; X052F/N/P; X66A/C/F/M/P/T/W; X067A/C/M; X069A/C/K; X073N/P; X077M/P/S; X079C/R/T; X080H/K/N; X081N/S; X084L/V; X092C/I/M; X094A/G/Y; X102G; X119A/D/G; X121G/L/M/P/V; X134Q/S/W; X140C/I/Q; X141F/K; X143G; X156C/P; X157A/I; X158A/Y; X164L/T; X165I/T/W; X166A/F/G/H/R; X172G/L; X192F/R; X203C/M/Q/W/Y; X210A/F/G/I/L/M/N/W; X213H/R; X214A/C/E/G/L/T/Y; X215C/D/F/H/R/V/W; X218F/A/H/K/Q/V/Y; X221M/R/T; X222C/M/V; X233M/PT/Y; X235F/Y; X236A/Q/S; G238H/M/N/S/T/V; X243A/P/S; X252F/H/M/T; X253K/V; X274A/D/K; X278A; X281D/P; F290M/V; X302F/H/K/M/P/Q/S/T/V/W; X310T/V/Y; X321D; X338I; X341M/T; X350T/C/E/I; X351E/V; X352D/N/S; X354L/M; X370M/N/R; X390D/E/L; X403G/K/R; X404K/M; X405Q/S/Y; X416C/Y; X418E/W; X422A/F/V; X430A/Q; X434S; X440G/H/L; X441L/S/W; X444L/P/S; X445M/Y; and/or X449L, where X is any amino acid corresponding to the equivalent position in SEQ ID NO:4 and/or an equivalent position in a parent glucoamylase or fragment thereof. In some embodiments, said one or more amino acid substitutions comprise one or more of S020A/E/F/G/P; K021M/S/T/W; E023L/M; E037C; S049W; A051K/L/V/Y; G052F/N/P; X66A/C/F/M/P/T/W; S067A/C/M; V069A/C/K; K073N/P; T077M/P/S; A079C/R/T; G080H/K/N; D081N/S; I084L/V; V092C/I/M; F094A/G/Y; X102G; S119A/D/G; T121G/L/M/P/V; E134Q/S/W; M140C/I/Q; L141F/K; A143G; F156C/P; T157A/I; N158A/Y; I164L/T; Y165I/T/W; K166A/F/G/H/R; V172G/L; Y192F/R; D203C/M/Q/W/Y; R210A/F/G/I/L/M/N/W; D213H/R; N214A/C/E/G/L/Y; S215C/D/F/H/R/V/W; A218F/A/H/K/Q/V/Y; S221M/R/T; G222C/M/V; S233M/PT/Y; W235F/Y; D236A/Q/S; G238H/M/N/S/T/V; T243A/P/S; V252F/H; E253K; G274A/D/K; P278A; E281D/P; F290M/V; N302F/H/K/M/P/Q/S/T/V/W; N310T/V/Y; N321D; F338I; L341M/T; K350C/E/I; N351E/V; T352D/N/S; V354L/M; S370M/N/R; S390D/E/L; Q403G/K/R; Y404K/M; H405Q/S/Y; F416C/Y; R418E/W; Y422A/F/V; T430A/Q; A434S; A440G/H/L; Q441L/S/W; A444L/P/S; G445M/Y; and/or F449L. In some embodiments of any of the embodiments disclosed herein, said variant comprises one or more amino acid substitutions at residue positions corresponding to positions 20, 21, 51, 79, 80, 92, 102, 121, 140, 143, 157, 158, 165, 166, 192, 203, 210, 213, 214, 215, 221, 222, 233, 235, 236, 238, 243, 252, 274, 278, 281, 290, 302, 310, 321, 341, 350, 352, 354, 370, 390, 403, 404, 405, 418, 440, 441, and/or 444 of SEQ ID NO:4 and/or an equivalent position in a parent glucoamylase, and wherein said variant glucoamylase exhibits improved hydrolysis of maltose compared to a parent glucoamylase or fragment thereof lacking one or more of these substitutions. In some embodiments, said one or more amino acid substitutions comprise one or more of X020A/P; X021S; X051L/V; X079C/T; X080K; X092M; X102G; X121G/P/M; X140I/C; X140C; X143G, X157I; X158A; X165W; X166G/R/F; X192F; X203W; X210L; X213R; X214A/E; X215D/C/F; X221R/T/M; X222M/C; X233M/Y/P; X235Y; X236A/Q; X238S/H; X243E/V; X252C/F; X274A; X278A; X281D; X290M/V; X302K/V/P/S/W; X310T/Y; X321D, X341M; X350I/T; X352D; X354L/M; X370M; X390E; X403G/R; X404K; X405Q/S/Y; X418W/E; X440G/H; X441S; and/or X444L, where X is any amino acid corresponding to the equivalent position in SEQ ID NO:4 and/or an equivalent position in a parent glucoamylase or fragment thereof. In some embodiments, said one or more amino acid substitutions comprise one or more of S020A/P; K021S; A051L/V; A079C/T; G080K; V092M; X102G; T121G/P/M; M140I/C; M140C; A143G; T157I; N158A; Y165W; K166G/R/F; Y192F; D203W; R210L; D213R; N214A/E; S215D/C/F; S221R/T/M; G222M/C; S233M/Y/P; W235Y; D236A/Q; G238S/H; T243E/V; V252C/F; G274A; P278A; E281D; F290M/V; N302K/V/P/S/W; N310T/Y; N321D; L341M; K350I/T; T352D; V354L/M; S370M; S390E; Q403G/R; Y404K; H405Q/S/Y; R418W/E; A440G/H; Q441S; and/or A444L. In some embodiments of any of the embodiments disclosed herein, said variant comprises one or more amino acid substitutions at residue positions corresponding to positions 20, 21, 23, 37, 51, 52, 67, 69, 73, 77, 79, 80, 81, 84, 92, 94, 102, 119, 121, 140, 141, 143, 164, 165, 166, 172, 210, 213, 214, 215, 218, 221, 222, 233, 236, 238, 252, 253, 274, 281, 290, 302, 321, 341, 350, 351, 352, 370, 390, 403, 404, 405, 418, 430, 440, 444, and/or 445 of SEQ ID NO:4 and/or an equivalent position in a parent glucoamylase, and wherein said variant glucoamylase exhibits improved hydrolysis of panose compared to a parent glucoamylase or fragment thereof lacking one or more of these substitutions. In some embodiments, said one or more amino acid substitutions comprise one or more of X020P/A; X021T/W/M; X023M/L; X037C; X051L/V; X052N; X067C; X069C/A; X073N; X077S; X079C, X080H/K; X081N; X084V/L; X092C; X094Y; X102G; X119G; X121M/L/G/P; X140C/Q/I; X141F; X143G, X164L; X165W/T; X166R; X172G; X210L; X213H; X214A/E; X215D; X218Q/A/V/Y; X221R; X222M/V/C; X233M/T/P; X236Q; X238H; X252F; X274D/K; X281D; X290M/V; X302S/K/W/Q/V; X321D, X341M/T; X350C/T; X351E; X352D/N; X370M; X390D/L/E; X403G; X403R/K; X404M/K; X405S/Q; X418E/W; X430A; X440G; X444P; and/or X445Y/M where X is any amino acid corresponding to the equivalent position in SEQ ID NO:4 and/or an equivalent position in a parent glucoamylase or fragment thereof. In some embodiments, said one or more amino acid substitutions comprise one or more of S020P/A; K021T/W/M; E023M/L; E037C; A051L/V; G052N; S067C; V069C/A; K073N; T077S; A079C; G080H/K; D081N; 1084V/L; V092C; F094Y; X102G; S119G; T121M/L/G/P; M140C/Q/I; L141F; A143G; 1164L; Y165W/T; K166R; V172G; R210L; D213H; N214A/E; S215D; S218Q/A/V/Y; S221R; G222M/V/C; S233M/T/P; D236Q; G238H; V252F; G274D/K; E281D; F290M/V; N302S/K/W/Q/V; N321D; L341M/T; K350C/T; N351E; T352D/N; S370M; S390D/L/E; Q403G; Q403R/K; Y404M/K; H405S/Q; R418E/W; T430A; A440G; A444P; and/or G445Y/M. In some embodiments of any of the embodiments disclosed herein, said variant comprises one or more amino acid substitutions at residue positions corresponding to positions 20, 51, 69, 73, 77, 79, 80, 81, 84, 94, 119, 121, 134, 140, 141, 143, 156, 158, 165, 166, 203, 210, 214, 215, 218, 221, 222, 233, 235, 236, 252, 253, 274, 278, 281, 290, 302, 310, 321, 341, 350, 352, 354, 370, 390, 416, 418, 434, 440, and/or 445, of SEQ ID NO:4 and/or an equivalent position in a parent glucoamylase, and wherein said variant glucoamylase exhibits improved hydrolysis of pullulan compared to a parent glucoamylase or fragment thereof lacking one or more of these substitutions. In some embodiments, said one or more amino acid substitutions comprise one or more of X020F/A/G; X051K; X069C; X073P/N; X077M/P; X079C/T; X080K; X081N; X084V/L; X094G/A; X119G/D; X121L/M; X134S; X140I/C/Q; X141F; X143G; X156P/C; X158A; X165T; X166H/A/F; X203C/Q/W/Y; X210L/M/F/N/A/I; X214A/Y/L/C; X215V/C/W/D/H; X218V/K/A/Q; X221R; X222M; X233M/P; X235Y; X236Q; X252F/H; X253K; X274K/D; X278A; X281D; X290M; X302P/S/V; X310T; X321D; X341M; X350I/T; X352D/S; X354L; X370M; X390D/L/E; X404K; X416Y; X418E/W; X434S; X440G; and/or X445M, where X is any amino acid corresponding to the equivalent position in SEQ ID NO:4 and/or an equivalent position in a parent glucoamylase or fragment thereof. In some embodiments, said one or more amino acid substitutions comprise one or more of S020F/A/G; A051K; V069C; K073P/N; T077M/P; A079T; G080K; D081N; 1084V/L; F094G/A; S119G/D; T121L/M; E134S; M140I/C/Q; L141F; A143G; F156P/C; N158A; Y165T; K166H/A/F; D203C/Q/W/Y; R210L/M/F/N/A/I; N214A/Y/L/C; S215V/C/W/D/H; S218V/K/A/Q; S221R; G222M; S233M/P; W235Y; D236Q; V252F/H; E253K; G274K/D; P278A; E281D; F290M; N302P/S/V; N310T; N321D; L341M; K350I/T; T352D/S; V354L; S370M; S390D/L/E; Y404K; F416Y; R418E/W; A434S; A440G; and/or G445M. In some embodiments of any of the embodiments disclosed herein, said variant comprises one or more amino acid substitutions at residue positions corresponding to positions 21, 23, 37, 52, 66, 69, 73, 77, 79, 80, 81, 84, 92, 119, 121, 134, 140, 141, 157, 158, 164, 165, 210, 213, 214, 218, 221, 222, 233, 235, 236, 243, 252, 253, 274, 281, 290, 302, 341, 350, 351, 352, 370, 390, 403, 404, 405, 416, 418, 422, 440, 441, 444, and/or 445 of SEQ ID NO:4 and/or an equivalent position in a parent glucoamylase or fragment thereof, and wherein said variant glucoamylase exhibits a higher performance index (PI) for the saccharification of soluble starch compared to a parent glucoamylase lacking one or more of these substitutions. In some embodiments, said one or more amino acid substitutions comprise one or more of X021T; X023M/L; X037C; X052N/F; X066A/F/T; X069C; X073N/P; X077P/M/S; X079R; X080N/H/K; X081N/S; X084L/V; X092I; X119A/G; X121M/L; X134S/Q; X140C/I/Q; X141F/K; X157A; X158A; X164L/T; X165W/T; X210W/G; X213H; X214G/Y; X218Q/F/A; X221R; X222M/V/C; X233M/P; X235F; X236Q; X243A; X252F; X253K; X274D/K/A; X281D; X290M/V; X302W/S/Q/K; X341M/T; X350C/E; X351E; X352D/N; X370M; X390D/L; X403G/R/K; X404I/M/K; X405S; X416C/Y; X418E/W; X422A; X440G; X441L; X444S/P; and/or X445M, where X is any amino acid corresponding to the equivalent position in SEQ ID NO:4 and/or an equivalent position in a parent glucoamylase or fragment thereof. In some embodiments, said one or more amino acid substitutions comprise one or more of K021T; E023M/L; E037C; G052N/F; V066A/F/T; V069C; K073N/P; T077P/M/S; A079R; G080N/H/K; D081N/S; 1084L/V; V092I; S119A/G; T121M/L; E134S/Q; M140C/I/Q; L141F/K; T157A; N158A; 1164L/T; Y165W/T; R210W/G; D213H; N214G/Y; S218Q/F/A; S221R; G222M/V/C; S233M/P; W235F; D236Q; T243A; V252F; E253K; G274D/K/A; E281D; F290M/V; N302W/S/Q/K; L341M/T; K350C/E; N351E; T352D/N; S370M; S390D/L; Q403G/R/K; Y404M/K; H405S; F416C/Y; R418E/W; Y422A; A440G; Q441L; A444S/P; and/or G445M. In some embodiments of any of the embodiments disclosed herein, said variant comprises one or more amino acid substitutions at residue positions corresponding to positions 20, 21, 51, 67, 69, 73, 77, 79, 80, 81, 84, 102, 119, 121, 134, 140, 141, 143, 156, 157, 158, 165, 166, 172, 192, 203, 210, 213, 214, 215, 218, 221, 222, 233, 235, 236, 252, 274, 281, 290, 302, 310, 321, 341, 350, 351, 352, 354, 370, 390, 403, 404, 405, 418, 422, 434, 440, 444, and/or 445 of SEQ ID NO:4 and/or an equivalent position in a parent glucoamylase, and wherein said variant glucoamylase exhibits improved hydrolysis of maltodextrin compared to a parent glucoamylase or fragment thereof lacking one or more of these substitutions. In some embodiments, said one or more amino acid substitutions comprise one or more of X020P/A/E; X021S/M; X051V/L/K/Y; X067A; X069C; X073P/N; X077P/M; X079C/T; X080K/H; X081N; X084L/V; X102G; X119G; X121M/G/V/P; X134S/Q; X140C/I; X141F; X143G; X156P/C; X157I; X158A; X165W/T; X166G; X172G; X192F; X203W/C/M; X210L/M; X213R; X214A/E; X215D/C/F; X218V/Y/Q; X221R/T; X222M/C; X233M/P/Y; X235Y; X236Q/A; X252F; X274K/A/D; X281D; X290M/V; X302P/K/S/V/Q/W; X310T/Y; X341M; X350I/T; X351V; X352D; X354L/M; X370M; X390E/L/D; X403K/R/G; X404K/M; X405Q/S/Y; X418W/E; X422A; X434S; X440G/H; X444P; and/or X445M, where X is any amino acid corresponding to the equivalent position in SEQ ID NO:4 and/or an equivalent position in a parent glucoamylase or fragment thereof. In some embodiments, said one or more amino acid substitutions comprise one or more of S020P/A/E; K021S/M; A051V/L/K/Y; S067A; V069C; K073P/N; T077P/M; A079C/T; G080K/H; D081N; 1084L/V; P102G; S119G; T121M/G/V/P; E134S/Q; M140C/I; L141F; A143G; F156P/C; T157I; N158A; Y165W/T; K166G; V172G; Y192F; D203W/C/M; R210L/M; D213R; N214A/E; S215D/C/F; S218V/Y/Q; S221R/T; G222M/C; S233M/P/Y; W235Y; D236Q/A; V252F; G274K/A/D; E281D; F290M/V; N302P/K/S/V/Q/W; N310T/Y; N321D; L341M; K350I/T; N351V; T352D; V354L/M; S370M; S390E/L/D; Q403K/R/G; Y404K/M; H405Q/S/Y; R418W/E; Y422A; A434S; A440G/H; A444P; and/or G445M. In some embodiments of any of the embodiments disclosed herein, said variant comprises one or more amino acid substitutions at residue positions corresponding to positions 23, 51, 52, 66, 67, 77, 119, 121, 134, 140, 141, 156, 157, 165, 192, 213, 221, 222, 233, 236, 252, 281, 290, 302, 350, 352, 370, 390, 403, 404, 416, and/or 422 of SEQ ID NO:4 and/or an equivalent position in a parent glucoamylase, and wherein said variant glucoamylase exhibits improved thermostability compared to a parent glucoamylase or fragment thereof lacking one or more of these substitutions. In some embodiments, said one or more amino acid substitutions comprise one or more of X023M; X051Y; X052N; X066A/C/F/M/W; X067C; X077P; X119A; X121M; X134S; X140I/C; X141F; X156C; X157I; X165W/I; X192F; X213R; X221R; X222M; X233M; X236Q; X252F; X281D; X290V; X302H/Q/W/S/K; X350E/C; X352D; X370M; X390L/D; X403R; X404K/M; X416Y; and/or X422A/V, where X is any amino acid corresponding to the equivalent position in SEQ ID NO:4 and/or an equivalent position in a parent glucoamylase or fragment thereof. In some embodiments, said one or more amino acid substitutions comprise one or more of E023M; A051Y; G052N; V066A/C/F/M/W; S067C; T077P; S119A; T121M; E134S; M140I/C; L141F; F156C; T157I; Y165W/I; Y192F; D213R; S221R; G222M; S233M; D236Q; V252F; E253F; E281D; F290V; N302H/Q/W/S/K; K350E/C; T352D; S370M; S390L/D; Q403R; Y404K/M; F416Y; and/or Y422A/V. In some embodiments of any of the embodiments disclosed herein, said variant comprises one or more amino acid substitutions at residue positions corresponding to positions 49, 51, 52, 66, 69, 77, 80, 94, 119, 134, 158, 164, 165, 172, 192, 213, 215, 218, 222, 233, 235, 236, 238, 243, 253, 274, 302, 338, 403, 405, 416, 418, 422, 430, 440, 441, 444, 445, and/or 449 of SEQ ID NO:4 and/or an equivalent position in a parent glucoamylase, and wherein said variant glucoamylase exhibits less reversion to saccharides having a degree of polymerization (DP) greater than or equal to 2 compared to a parent glucoamylase or fragment thereof lacking one or more of these substitutions. In some embodiments, said one or more amino acid substitutions comprise one or more of X049W; X051Y; X052P; X66P; X069K/C; X077P/M; X080H; X094A/G; X119D; X134W/Q; X158Y; X164T/L; X165W/I; X172G; X192R/F; X213H; X215R; X218K; X222C; X233T/P; X235F; X236A; X238H/S/N; X243P/S; X253K; X274K/A; X302H/F/P/M/Q/T; X338I; X403G; X405Q/S; X416C/Y; X416Y; X418W/E; X422A; X430A/Q; X440L/H; X441W/L; X444L/P; X445Y; and/or X449L, where X is any amino acid corresponding to the equivalent position in SEQ ID NO:4 and/or an equivalent position in a parent glucoamylase or fragment thereof. In some embodiments, said one or more amino acid substitutions comprise one or more of S049W; A051Y; G052P; V66P; V069K/C; T077P/M; G080H; F094A/G; S119D; E134W/Q; N158Y; 1164T/L; Y165W/I; V172G; Y192R/F; D213H; S215R; S218K; G222C; S233T/P; W235F; D236A; G238H/S/N; T243P/S; E253K; G274K/A; N302H/F/P/M/Q/T; F338I; Q403G; H405Q/S; F416C/Y; F416Y; R418W/E; Y422A; T430A/Q; A440L/H; Q441W/L; A444L/P; G445Y; and/or F449L. In some embodiments of any of the embodiments disclosed herein, said variant comprises one or more amino acid substitutions at residue positions corresponding to positions 21, 23, 51, 52, 66, 67, 79, 81, 92, 119, 140, 158, 164, 172, 192, 210, 213, 214, 215, 218, 221, 222, 233, 235, 236, 238, 243, 253, 281, 290, 302, 310, 351, 352, 354, 370, 403, 416, 422, 430, 441, and/or 445 of SEQ ID NO:4 and/or an equivalent position in a parent glucoamylase, and wherein said variant glucoamylase exhibits improved saccharification yield of glucose compared to a parent glucoamylase lacking one or more of these substitutions. In some embodiments, said one or more amino acid substitutions comprise one or more of X021S/W; X023L; X051Y/K; X052N; X066/A/C/F/M/W; X067M/C/A; X079R; X081S; X092C/M; X119A; X140I; X158A/Y; X164L; X172L/G; X192F; X210W; X213H; X214Y; X215F/C/R; X218H/K/A; X221M/T; X222V; X233T; X235Y/F; X236S/A; X238T/V/M; X243A; X253K; X281P/D; X290V; X302K/Q/H/W; X310V/T; X351V; X352S; X354L; X370R; X403K; X416Y; X422F; X430A; X441L; and/or X445Y, where X is any amino acid corresponding to the equivalent position in SEQ ID NO:4 and/or an equivalent position in a parent glucoamylase. In some embodiments, said one or more amino acid substitutions comprise one or more of K021S/W; E023L; A051Y/K; G052N; V066/A/C/F/M/W; S067M/C/A; A079R; D081S; V092C/M; S119A; M140I; N158A/Y; I164L; V172L/G; Y192F; R210W; D213H; N214Y; S215F/C/R; S218H/K/A; S221M/T; G222V; S233T; W235Y/F; D236S/A; G238T/V/M; T243A; E253K; E281P/D; F290V; N302K/Q/H/W; N310V/T; N351V; T352S; V354L; S370R; Q403K; F416Y; Y422F; T430A; Q441L; and/or G445Y. In some embodiments of any of the embodiments disclosed herein, said variant comprises one or more amino acid substitutions at residue positions corresponding to positions 20, 21, 23, 37, 51, 52, 66, 67, 69, 73, 77, 79, 80, 81, 84, 92, 94, 102, 119, 121, 134, 140, 141, 156, 157, 158, 164, 165, 166, 172, 192, 203, 210, 213, 214, 215, 218, 221, 222, 233, 235, 236, 238, 243, 252, 253, 274, 278, 281, 290, 302, 310, 341, 350, 351, 352, 354, 370, 390, 403, 404, 405, 416, 418, 422, 430, 440, 441, 444, and/or 445, of SEQ ID NO:4 and/or an equivalent position in a parent glucoamylase or fragment thereof, and wherein said variant glucoamylase exhibits two or more of i) improved hydrolysis of maltose; ii) improved hydrolysis of panose; iii) improved hydrolysis of pullulan; iv) improved hydrolysis of soluble starch; v) improved hydrolysis of maltodextrin; vi) improved thermostability; vii) less reversion to saccharides having a DP greater than or equal to 2; and/or viii) improved saccharification yield of glucose compared to a parent glucoamylase lacking one or more of these substitutions. In some embodiments, said one or more amino acid substitutions comprise one or more of X020A/P; X021M/S/T/W; X023L/M; X037C; X051K/L/V/Y; X052N; X066A/C/F/M/W; X067A/C; X069C; X073N/P; X077M/P/S; X079R/T; X080H/K; X081N/S; X084L/V; X092C/M; X094A/G; X102G; X119A/D/G; X121G/L/M/P; X134Q/S; X140C/I/Q; X141F; X156C/P; X157I; X158A/Y; X164L/T; X165I/T/W; X166G/F/R; X172G; X192F; X203C/W; X210L/M/W; X213H/R; X214A/E/Y; X215C/D/F/R; X218A/K/Q/V/Y; X221M/R/T; X222C/M/V; X233M/P/T/Y; X235F/Y; X236A/Q; X238H/S; X243A; X252F; X253K; X274A/D/K; X278A; X281D; X290M/V; X290V; X302H/K/P/Q/S/V/W; X310T/Y; X341M/T; X350C/E/I; X351E/V; X352D/N/S; X354L/M; X370M; X390D/E/L; X403G/K/R; X404K/M; X405Q/S/Y; X416C/Y; X418E/W; X422A; X430A; X440G/H; X441L; X444L/P; and/or X445M/Y, where X is any amino acid corresponding to the equivalent position in SEQ ID NO:4 and/or an equivalent position in a parent glucoamylase or fragment thereof. In some embodiments, said one or more amino acid substitutions comprise one or more of S020A/E/P; K021M/S/T/W; E023L/M; E037C; A051K/L/V/Y; G052N; V066A/C/F/M/W; S067A/C; V069C; K073N/P; T077M/P/S; A079R/T; G080H/K; D081N/S; 1084L/V; V092C/M; F094A/G; P102G; S119A/D/G; T121G/L/M/P; E134Q/S; M140C/I/Q; L141F; F156C/P; T157I; N158A/Y; 1164L/T; Y165I/T/W; K166G/F/R; V172G; Y192F; D203C/W; R210L/M/W; D213H/R; N214A/E/Y; S215C/D/F/R; S218A/K/Q/V/Y; S221M/R/T; G222C/M/V; S233M/P/T/Y; W235F/Y; D236A/Q; G238H/S; T243A; V252F; E253K; G274A/D/K; P278A; E281D; F290M/V; F290V; N302H/K/P/Q/S/V/W; N310T/Y; L341M/T; K350C/E/I; N351E/V; T352D/N/S; V354L/M; S370M; S390D/E/L; Q403G/K/R; Y404K/M; H405Q/S/Y; F416C/Y; R418E/W; Y422A; T430A; A440G/H; Q441L; A444L/P; and/or G445M/Y. In some embodiments of any of the embodiments disclosed herein, said variant comprises two or more amino acid substitutions comprising a) X236S and X281D; b) X215R and X441W; c) X321D and X434S; d) X143G and X434S; e) X079C and X143G; f) X350T and X434S; g) X351E and X403K; h) X052N and X084L; i) X243P and X290V; j) X290V and X350E; k) X302K and X441W; 1) X156C and X404K; m) X067M and X404K; n) X052N and X141F; o) X052N and X351E; p) X233M and X445Y; q) X066C and X233M; r) X218H and X290V; s) X067M and X302H; t) X066C and X119A; u) X243P and X445Y; v) X192F and X243P; w) X156C and X243P; x) X023L and X066C; y) X023L-X119A; z) X020E and X192F; aa) X192F and X310V; bb) X023M and X302H; cc) X192F and X416Y; dd) X119A and X302H; ee) X235Y and X416Y; ff) X052N and X404K; gg) X023M and X449Y; hh) X158A and X172L; ii) X172L and X290V; jj) X023M and X210L; kk) X210L and X449Y; 11) X157I and X281D; mm) X164T and X215R; nn) X140C and X422V; oo) X119A and X302K; or pp) X052N and X416Y, where X is any amino acid corresponding to the equivalent position in SEQ ID NO:4 and/or an equivalent position in a parent glucoamylase or fragment thereof. In some embodiments, said two or more amino acid substitutions comprise a) D236S and E281D; b) S215R and Q441W; c) N321D and A434S; d) A143G and A434S; e) A079C and A143G; f) K350T and A434S; g) N351E and Q403K; h) G052N and I084L; i) T243P and F290V; j) F290V and K350E; k) N302K and Q441W; 1) F156C and Y404K; m) S067M and Y404K; n) G052N and L141F; o) G052N and N351E; p) S233M and G445Y; q) A066C and S233M; r) S218H and F290V; s) S067M and N302H; t) A066C and S119A; u) T243P and G445Y; v) Y192F and T243P; w) F156C and T243P; x) E023L and A066C; y) E023L and S119A; z) S020E and Y192F; aa) Y192F and N310V; bb) E023M and N302H; cc) Y192F and F416Y; dd) S119A and N302H; ee) W235Y and F416Y; ff) G052N and Y404K; gg) E023M and F449Y; hh) N158A and V172L; ii) V172L and F290V; jj) E023M and R210L; kk) R210L and F449Y; 11) T157I and E281D; mm) 1164T and S215R; nn) M140C and Y422V; oo) S119A and N302K; or pp) G052N and F416Y. In some embodiments of any of the embodiments disclosed herein, said variant comprises three or more amino acid substitutions comprising a) X141F, X281D, and X441W; b) X143G, X321D, and X434S; c) X321D, X350T, and X434S; d) X067M, X158A, and X281D; e) X156C, X192F, and X403K; f) X052N, X140C, and X422V; g) X066C, X119A, and X164T; h) X066C, X233M, and X445Y; i) X156C, X192F, and X243P; j) X023L, X066C, and X119A; k) X023M, X119A, and X404K; 1) X310V, X416Y, and X445Y; m) X158A, X221R, and X290V; n) X023M, X052N, and X404K; o) X081S, X157I, and X236S; p) X243P, X302K, and X416Y; q) X140C, X302K, and X422V; or r) X052N, X416Y, and X445Y, where X is any amino acid corresponding to the equivalent position in SEQ ID NO:4 and/or an equivalent position in a parent glucoamylase or fragment thereof. In some embodiments, said three or more amino acid substitutions comprise a) L141F, E281D, and Q441W; b) A143G, N321D, and A434S; c) N321D, K350T, and A434S; d) S067M, N158A, and E281D; e) F156C, Y192F, and Q403K; f) G052N, M140C, and Y422V; g) A066C, S119A, and I164T; h) A066C, S233M, and G445Y; i) F156C, Y192F, and T243P; j) E023L, A066C, and S119A; k) E023M, S119A, and Y404K; 1) N310V, F416Y, and G445Y; m) N158A, S221R, and F290V; n) E023M, G052N, and Y404K; o) D081S, T157I, and D236S; p) T243P, N302K, and F416Y; q) M140C, N302K, and Y422V; or r) G052N, F416Y, and G445Y. In some embodiments of any of the embodiments disclosed herein, said variant comprises four or more amino acid substitutions comprising a) X215R, X236S, X281D, and X441W; b) X052N, X084L, X140C, and X422V; c) X020E, X156C, X192F, and X243P; d) X023M, X221R, and X404K; e) X158A, X172L, X221R, and X290V; f) X140C, X165W, X302K, and X422V; or g) X119A, X253F, X310V, and X403K, where X is any amino acid corresponding to the equivalent position in SEQ ID NO:4 and/or an equivalent position in a parent glucoamylase or fragment thereof. In some embodiments, said four or more amino acid substitutions comprise a) S215R, D236S, E281D, and Q441W; b) G052N, I084L, M140C, and Y422V; c) S020E, F156C, Y192F, and T243P; d) E023M, S119A, S221R, and Y404K; e) N158A, V172L, S221R, and F290V; f) M140C, Y165W, N302K, and Y422V; or g) S119A, E253F, N310V, and Q403K. In some embodiments of any of the embodiments disclosed herein, said variant comprises five or more amino acid substitutions comprising X067M, X157I, X218H, X302H, and X416Y, where X is any amino acid corresponding to the equivalent position in SEQ ID NO:4 and/or an equivalent position in a parent glucoamylase or fragment thereof. In some embodiments, said five or more amino acid substitutions comprise S067M, T157I, S218H, N302H, and F416Y. In some embodiments of any of the embodiments disclosed herein, said variant glucoamylase exhibits one or more of i) improved hydrolysis of maltose; ii) improved hydrolysis of panose; iii) improved hydrolysis of pullulan; iv) a higher PI for the saccharification of soluble starch (i.e. improved hydrolysis of soluble starch or a fragment thereof); v) improved hydrolysis of maltodextrin; vi) improved thermostability; vii) less reversion to saccharides having a DP greater than or equal to 2; and/or viii) improved saccharification yield of glucose compared to a parent glucoamylase lacking these substitutions. In some embodiments of any of the embodiments disclosed herein, said variant comprises substitutions at residue positions corresponding to positions a) 66, 67, and 69; b) 102, 119, and 121; and/or c) 143, 156, 164, 192, and 233 of SEQ ID NO: 3, SEQ ID NO:4 and/or an equivalent position in a parent glucoamylase or fragment thereof. In some embodiments, said variant comprises amino acid substitutions comprising a) X066A/C/F/M/P/T/W, X067A/C/M, and X069A/C/K; b) X102P/G, X119A/D/G, X121G/L/M/P/V; and/or c) X143G, X156C/P, X164L/T, X192F/R, and X233M/PT/Y. In some embodiments, said variant comprises amino acid substitutions comprising a) V066A/C/F/M/P/T/W, S067A/C/M, and V069A/C/K; b) S102P/G, S119A/D/G, T121G/L/M/P/V; and/or c) A143G, F156C/P, I164L/T, Y192F/R, and S233M/PT/Y. In some embodiments of any of the embodiments disclosed herein, the glucoamylase variant further comprises an amino acid substitution at one or more residue positions corresponding to positions 66 and/or 102 of SEQ ID NO:4 and/or an equivalent position in a parent glucoamylase. In some embodiments, said one or more amino acid substitutions comprise X066A/C/F/M/P/T/W and/or X102P/G, where X is any amino acid corresponding to the equivalent position in SEQ ID NO:4 and/or an equivalent position in a parent glucoamylase. In some embodiments, said one or more amino acid substitutions comprise V066A/C/F/M/P/T/W and/or S102P/G. In some embodiments of any of the embodiments disclosed herein, the glucoamylase variant comprises an N-linked glycosylation at position N075 of SEQ ID NO:4 and/or an equivalent position in a parent glucoamylase. In some embodiments of any of the embodiments disclosed herein, the glucoamylase variant further comprises one or more amino acid substitutions at residue positions corresponding to positions 81, 83, 153, 370 or 372 of SEQ ID NO:4 and/or an equivalent position in a parent glucoamylase, and wherein said variant glucoamylase exhibits increased glycosylation and increased thermostability compared to a parent glucoamylase lacking one or more of these substitutions. In some embodiments, said one or more substitutions comprise one or more substitutions at position D81N, K83T, A153T, S370N, and/or A372S.

In other aspects, provided herein is a polynucleotide encoding any of the glucoamylase variant polypeptides disclosed herein.

In another aspect, provided herein is a vector comprising any of the polynucleotides disclosed herein.

In further aspects, provided herein is a host cell comprising any of the polynucleotides disclosed herein or any of the vectors disclosed herein. In some embodiments, the host cell is a bacterial or fungal host cell. In some embodiments of any of the embodiments disclosed herein, the host cell is an Aspergillus spp. a Bacillus spp., or a Trichoderma spp., a Pichia spp., a Myceliophthora spp., or a Saccharomyces spp.

In other aspects, provided herein is an enzyme composition comprising any of the glucoamylase variants disclosed herein. In some embodiments of any of the embodiments disclosed herein, said composition is used in a starch conversion process. In some embodiments of any of the embodiments disclosed herein, said composition is used in an animal feed formulation. In some embodiments of any of the embodiments disclosed herein, said composition is used in an alcohol fermentation process. In some embodiments of any of the embodiments disclosed herein, said composition is used in a process to make a fermented beverage.

In still additional aspects, provided herein is a method of producing a variant glucoamylase in a host cell comprising a) culturing a host cell transformed with any of the vectors disclosed herein in a culture medium under conditions suitable for production of said glucoamylase variant; and b) producing said variant. In some embodiments, the method further comprises recovering the glucoamylase variant from said culture medium. In some embodiments of any of the embodiments disclosed herein, the host cell is a bacterial or fungal host cell. In some embodiments of any of the embodiments disclosed herein, the host cell is an Aspergillus spp. a Bacillus spp., or a Trichoderma spp., a Pichia spp., a Myceliophthora spp., or a Saccharomyces spp.

In another aspect, provided herein is a method of saccharifying a composition comprising starch to produce a composition comprising glucose, wherein said method comprises: a) contacting a starch composition with any of the glucoamylase variant polypeptides disclosed herein; and b) saccharifying the starch composition to produce said glucose composition. In some embodiments, said composition comprising starch comprises liquefied starch, gelatinized starch, or granular starch. In some embodiments of any of the embodiments disclosed herein, the method further comprises c) contacting the starch composition with an alpha-amylase. In some embodiments of any of the embodiments disclosed herein, the method further comprises d) contacting the starch composition with a pullulanase. In some embodiments of any of the embodiments disclosed herein, the method further comprises e) fermenting the glucose composition to produce a fermentation product. In some embodiments, the fermentation product is an alcohol. In some embodiments, the alcohol is ethanol or butanol. In some embodiments of any of the embodiments disclosed herein, the method further comprises f) adding one or more of an additional glucoamylase, hexokinase, xylanase, glucose isomerase, xylose isomerase, phosphatase, phytase, protease, pullulanase, β-amylase, an additional α-amylase, protease, cellulase, hemicellulase, lipase, cutinase, trehalase, isoamylase, redox enzyme, esterase, transferase, pectinase, alpha-glucosidase, beta-glucosidase, lyase, hydrolase, or a combination thereof, to said starch composition. In some embodiments of any of the embodiments disclosed herein, the fermentation is a simultaneous saccharification and fermentation (SSF) reaction.

Each of the aspects and embodiments described herein are capable of being used together, unless excluded either explicitly or clearly from the context of the embodiment or aspect.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### BRIEF DESCRIPTION OF THE DRAWINGS

FIG. 1 provides a multiple amino acid sequence alignment of Mucorales-clade glucoamylases.

FIG. 2 provides a phylogenetic tree of predicted mature Mucorales-clade glucoamylases and other fungal glucoamylases.

FIG. 3 provides an image of the electron density (2fo-fc) shown at the site of Asn75 glycosylation based on the x-ray diffraction structure determined for SvaGA1v2.

FIG. 4 provides and image of the interactions of the glycan chain of the residue Asn75 with the protein based on the x-ray diffraction structure determined for SvaGA1v2. Hydrogen bonding interactions (<3.3 Å) indicated with dashed lines.

FIG. 5 provides and image where highlighted in dark gray are the helix containing the conserved Asp63 side chain as well as performance-improving mutations at positions 66, 67, and 69 based on the x-ray diffraction structure determined for SvaGA1v2.

FIG. 6 provides and image where highlighted in black are the loop at residues 100-129, the expected general acid residue, the conserved Asp side chain, and the amino acid positions of performance-improving mutations, based on the x-ray diffraction structure determined for SvaGA1v2.

FIG. 7 provides an image where the four-helix bundle associated with the glycan chain of residue Asn75 is highlighted in dark gray, while the amino acid positions of several performance-improving mutations are shown in black spheres, based on the x-ray diffraction structure determined for SvaGA1v2. The Asn75 glycan is shown with a stick model colored in black.

### DETAILED DESCRIPTION

The present disclosure relates to a recombinant host cell, a composition comprising a glucoamylase and methods of saccharifying the starch substrate using the glucoamylase. Moreover, the disclosure also relates to a process of producing fermentation products and a method for increasing starch digestibility in an animal as well as a method of producing a fermented beverage.

### I. Definitions

Prior to describing the compositions and methods in detail, the following terms and abbreviations are defined.

Unless otherwise defined, all technical and scientific terms used have their ordinary meaning in the relevant scientific field. Singleton, et al., Dictionary of Microbiology and Molecular Biology, 2d Ed., John Wiley and Sons, New York (1994), and Hale & Markham, Harper Collins Dictionary of Biology, Harper Perennial, NY (1991) provide the ordinary meaning of many of the terms describing the invention.

The term “glucoamylase (1,4-alpha-D-glucan glucohydrolase, EC 3.2.1.3) activity” is defined herein as an enzyme activity, which catalyzes the release of D-glucose from the non-reducing ends of starch or related oligo- and poly-saccharide molecules.

The term “glucoamylase variant,” as used herein, means a non-naturally occurring glucoamylase having at least one (such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50) amino acid substitution in a given parent glucoamylase amino acid sequence.

The term, “wild-type,” with respect to a polypeptide (such as a glucoamylase), refers to a naturally-occurring polypeptide that does not include a human-made substitution, insertion, or deletion at one or more amino acid positions. However, in another embodiment, non-limiting examples of wild-type glucoamylases include SEQ ID NOs: 4-37.

The terms, “parent, “parental,” or “reference” with respect to a polypeptide (such as a glucoamylase), can refer to a wild-type polypeptide or can also refer to a polypeptide that has had one or more amino acid substitutions introduced into it which is then used as a reference to compare performance characteristics of a polypeptide that has had further amino acid substitutions (such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 amino acid substitution) into it. In some embodiments, the parent polypeptide is SEQ ID NOs: 2, 3, 5, or 6. In other embodiments, the parent polypeptide is SEQ ID NOs: 4-37.

The term “amino acid sequence” is synonymous with the terms “polypeptide”, “protein” and “peptide” and are used interchangeably. Where such amino acid sequences exhibit activity, they may be referred to as an “enzyme”. The conventional one-letter or three-letter codes for amino acid residues are used, with amino acid sequences being presented in the standard amino-to-carboxy terminal orientation (i.e., N→C).

The term “mature polypeptide” is defined herein as a polypeptide in its final form following translation and any post-translational modifications, such as N-terminal processing, C-terminal truncation, glycosylation, phosphorylation, etc. In one aspect, the predicted mature polypeptide is SEQ ID NO: 4 based on the analysis of SignalP software version 4.0 (Nordahl Petersen et al. (2011) Nature Methods, 8:785-786). In another aspect, the mature polypeptide comprises amino acid position 20-468 of SEQ ID NO:2. In another aspect, the mature polypeptide comprises amino acid position 21-468 of SEQ ID NO:2. In another aspect, the mature polypeptide comprises amino acid position 22-468 of SEQ ID NO:2. In another aspect, the mature polypeptide comprises amino acid position 23-468 of SEQ ID NO:2. In another aspect, the mature polypeptide comprises amino acid position 24-468 of SEQ ID NO:2. In another aspect, the mature polypeptide comprises amino acid position 25-468 of SEQ ID NO:2.

A “signal sequence” or “signal peptide” is a sequence of amino acids attached to the N-terminal portion of a protein, which facilitates the secretion of the protein outside the cell. The mature form of an extracellular protein lacks the signal sequence, which is cleaved off during the secretion process. In some embodiments, SEQ ID NO: 34 is a signal peptide. In other embodiments, the signal peptide comprises amino acid positions 1-20 of SEQ ID NO:2. In other embodiments, the signal peptide comprises amino acid positions 1-21 of SEQ ID NO:2. In other embodiments, the signal peptide comprises amino acid positions 1-22 of SEQ ID NO:2. In other embodiments, the signal peptide comprises amino acid positions 1-23 of SEQ ID NO:2. In other embodiments, the signal peptide comprises amino acid positions 1-24 of SEQ ID NO:2.

The term “nucleic acid” or “polynucleotide” can be used interchangeable to encompass DNA, RNA, heteroduplexes, and synthetic molecules capable of encoding a polypeptide. Nucleic acids may be single stranded or double stranded, and may be chemically modified. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

The term “coding sequence” means a polynucleotide sequence, which directly specifies the amino acid sequence of its protein product. The boundaries of the coding sequence are generally determined by an open reading frame, which usually begins with the ATG start codon or alternative start codons such as GTG and TTG and ends with a stop codon such as TAA, TAG, and TGA. The coding sequence may be a DNA, cDNA, synthetic, or recombinant nucleotide sequence.

The term “cDNA” is defined herein as a DNA molecule that can be prepared by reverse transcription from a mature, spliced, mRNA molecule obtained from a eukaryotic cell. cDNA lacks intron sequences that may be present in the corresponding genomic DNA. The initial, primary RNA transcript is a precursor to mRNA that is processed through a series of steps before appearing as mature spliced mRNA. These steps include the removal of intron sequences by a process called splicing. cDNA derived from mRNA lacks, therefore, any intron sequences.

A “synthetic” molecule is produced by in vitro chemical or enzymatic synthesis rather than by an organism.

A “host strain” or “host cell” is an organism into which an expression vector, phage, virus, or other DNA construct, including a polynucleotide encoding a polypeptide of interest (e.g., an glucoamylase) has been introduced. Exemplary host strains are microorganism cells (e.g., bacteria, filamentous fungi, and yeast) capable of expressing the polypeptide of interest and/or fermenting saccharides. The term “host cell” includes protoplasts created from cells.

The term “glycosylation” as used herein refers to the attachment of glycans to molecules, for example to proteins, such as variant glucoamylases. Glycosylation may be an enzymatic reaction. The attachment formed may be through covalent bonds. The phrase “highly glycosylated” refers to a molecule such as an enzyme which is glycosylated in many sites and at all or nearly all the available glycosylation sites, for instance N-linked glycosylation sites. Alternatively, or in addition to, the phrase “highly glycosylated” can refer to extensive glycolytic branching (such as, the size and number of glycolytic moieties associated with a particular N-linked glycosylation site) at all or substantially all N-linked glycosylation sites. In some embodiments, the engineered glucoamylase polypeptide is glycosylated at all or substantially all consensus N-linked glycosylation sites (i.e. an NXS/T consensus N-linked glycosylation site, wherein X is any amino acid except for proline). A glucoamylase may have varying degrees of glycosylation. It is known that such glycosylations may improve stability during storage and in applications. In further embodiments, one or more (such as 1, 2, 3, 4, 5, 6, 7, 8, 9, or 10 or more) consensus N-linked glycosylation sites (i.e. an NXS/T consensus N-linked glycosylation site, wherein X is any amino acid except for proline) can be introduced into a glucoamylase (such as a variant glucoamylase) to improve one or more property of the glucoamylase such as, without limitation, improved thermostability, activity, or saccharification yield.

The term “glycan” as used herein refers to a polysaccharide or oligosaccharide, or the carbohydrate section of a glycoconjugate such as a glycoprotein. Glycans may be homo- or heteropolymers of monosaccharide residues. They may be linear or branched molecules.

The term “expression” refers to the process by which a polypeptide is produced based on a nucleic acid sequence. The process includes both transcription and translation.

The term “vector” refers to a polynucleotide sequence designed to introduce nucleic acids into one or more cell types. Vectors include cloning vectors, expression vectors, shuttle vectors, plasmids, phage particles, cassettes and the like.

An “expression vector” refers to a DNA construct comprising a DNA sequence encoding a polypeptide of interest, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

The term “control sequences” is defined herein to include all components necessary for the expression of a polynucleotide encoding a polypeptide of the present invention. Each control sequence may be native or foreign to the nucleotide sequence encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide.

The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequences.

The term “sequence motif” is a nucleotide or amino-acid sequence pattern that is widespread and has been proven or assumed to have a biological significance. In this invention, the sequence motif is an amino-acid sequence motif identified in the Mucorales-clade glucoamylases.

“Biologically active” refer to a sequence having a specified biological activity, such an enzymatic activity.

The term “specific activity” refers to the number of moles of substrate that can be converted to product by an enzyme or enzyme preparation per unit time under specific conditions. Specific activity is generally expressed as units (U)/mg of protein.

The term “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%) In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A, 87:2264-2268, modified as in Karlin and Altschul, 1993. Proc. Natl. Acad. Sci. U.S.A. 90:5873-5877. Such an algorithm is incorporated into the NBLAST and XBLAST programs of Altschul et al., 1990, J. Mol. Biol 215:403. BLAST nucleotide searches can be performed with the NBLAST nucleotide program parameters set, e.g., for score=100, word length=12 to obtain nucleotide sequences homologous to a nucleic acid molecules of the present application. BLAST protein searches can be performed with the XBLAST program parameters set, e.g., to score-50, word length=3 to obtain amino acid sequences homologous to a protein molecule described herein. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Another computer program that can be used to create multiple alignments of protein sequences is MUSCLE. Elements of the MUSCLE algorithm include fast distance estimation using kmer counting, progressive alignment using a new profile function described as log-expectation score, and refinement using tree-dependent restricted partitioning. This program is described in MUSCLE: multiple sequence alignment with high accuracy and high throughput by Robert C. Edgar (2004) published in Nucleic Acids Res. 32: 1792-1797. The percent identity between two sequences can be determined using techniques similar to those described above, with or without Allowing gaps. In calculating percent identity, typically only exact matches are counted.

The term “homologous sequence” is defined herein as a predicted protein having an E value (or expectancy score) of less than 0.001 in a tfasty search (Pearson, W. R., 1999, in Bioinformatics Methods and Protocols, S. Misener and S. A. Krawetz, ed., pp. 185-219) with a glucoamylase, for example, the glucoamylase of SEQ ID NO: 4.

As used herein, an “equivalent position” means a position that is common to two amino acid sequences that is based on an alignment of the amino acid sequence of a parent glucoamylase with a glucoamylase variant as well as alignment of a three-dimensional structure of a parent glucoamylase with that of a variant glucoamylase in three-dimensional space.

As used herein with regard to amino acid residue positions, “corresponding to” or “corresponds to” or “correspond to” or “corresponds” refers to an amino acid residue at the enumerated position in a protein or peptide, or an amino acid residue that is analogous, homologous, or equivalent to an enumerated residue in a protein or peptide. As used herein, “corresponding region” generally refers to an analogous position in a related protein or a reference protein.

As used herein, “performance index” or “PI” refers to calculated activity per unit of an enzyme relative to a parent molecule. In some aspects of any of the embodiments disclosed herein, the parental molecule used in the calculation of the performance index is a glucoamylase. In some embodiments, the parental molecule has a performance index of one, by definition. In other embodiments, a performance index greater than one (PI>1.0) indicates improved activity of a glucoamylase variant compared to the parent molecule.

As used herein, a “reversion sugar” is a sugar that is formed when a monosaccharide condenses with another monosaccharide (occasionally, disaccharide) in the presence of a catalyst (e.g., acid) to form an oligosaccharide, such as (predominantly) a disaccharide or (rarely) a trisaccharide (in a process defined herein as “reversion”). As a result, in many cases the reversion sugar has a bonding linkage that is not present in the original starch composition. Examples of reversion sugars include, for example, xylobiose, (both α- and β-forms of (1,1), (1,2), (1,3), and (1,4)-linked xylobiose), O-α-D-xylopyranosyl-α-D-xylopyranoside, 3-O-α-D-xylopyranosyl-D-xylose, 2-O-α-D-xylopyranosyl-D-xylose, 4-O-α-D-xylopyranosyl-D-xylose, maltose, isomaltose, cellobiose, gentiobiose, 1,6-anhydro-β-D-glucofuranose, kojibiose, sophorose, nigerose, laminarabiose, and any combination thereof. Reversion sugars are typically non-fermentable and are thus considered waste products of sacharification reactions.

The phrase “simultaneous saccharification and fermentation (SSF)” refers to a process in the production of biochemicals in which a microbial organism, such as an ethanologenic microorganism, and at least one enzyme, such as an amylase, are present during the same process step. SSF includes the contemporaneous hydrolysis of starch substrates (granular, liquefied, or solubilized) to saccharides, including glucose, and the fermentation of the saccharides into alcohol or other biochemical or biomaterial in the same reactor vessel.

A “slurry” is an aqueous mixture containing insoluble starch granules in water.

The term “total sugar content” refers to the total soluble sugar content present in a starch composition including monosaccharides, oligosaccharides and polysaccharides.

The term “dry solids” (ds) refer to dry solids dissolved in water, dry solids dispersed in water or a combination of both. Dry solids thus include granular starch, and its hydrolysis products, including glucose.

The term “high DS” refers to aqueous starch slurry with a dry solid content greater than 38% (wt/wt).

“Degree of polymerization (DP)” refers to the number (n) of anhydroglucopyranose units in a given saccharide. Examples of DP1 are monosaccharides, such as glucose and fructose. Examples of DP2 are disaccharides, such as maltose and sucrose. A DP4+(>DP3) denotes polymers with a degree of polymerization of greater than 3.

The term “contacting” refers to the placing of referenced components (including but not limited to enzymes, substrates, and fermenting organisms) in sufficiently close proximity to affect an expect result, such as the enzyme acting on the substrate or the fermenting organism fermenting a substrate.

As used herein, the terms “yeast cells,” “yeast strains,” or simply “yeast” refer to organisms from the Ascomycota and Basidiomycota. Exemplary yeast is budding yeast from the order Saccharomycetales. Particular examples of yeast are Saccharomyces spp., including but not limited to S. cerevisiae. Yeast include organisms used for the production of fuel alcohol as well as organisms used for the production of potable alcohol, including specialty and proprietary yeast strains used to make distinctive-tasting beers, wines, and other fermented beverages.

An “ethanologenic microorganism” refers to a microorganism with the ability to convert a sugar or other carbohydrates to ethanol.

The term “biochemicals” refers to a metabolite of a microorganism, such as citric acid, lactic acid, succinic acid, monosodium glutamate, gluconic acid, sodium gluconate, calcium gluconate, potassium gluconate, glucono delta-lactone, sodium erythorbate, omega 3 fatty acid, butanol, iso-butanol, an amino acid, lysine, itaconic acid, other organic acids, 1,3-propanediol, vitamins, or isoprene or other biomaterial.

The term “pullulanase” also called debranching enzyme (E.C. 3.2.1.41, pullulan 6-glucanohydrolase), is capable of hydrolyzing alpha 1-6 glucosidic linkages in an amylopectin molecule.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. For example, in connection with a numerical value, the term “about” refers to a range of −15% to +15% of the numerical value, unless the term is otherwise specifically defined in context.

As used herein, the singular terms “a,” “an,” and “the” include the plural reference unless the context clearly indicates otherwise.

It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

The term “comprising” and its cognates are used in their inclusive sense; that is, equivalent to the term “including” and its corresponding cognates. It is further noted that the term “comprising,” as used herein, means including, but not limited to, the component(s) after the term “comprising.” The component(s) after the term “comprising” are required or mandatory, but the composition comprising the component(s) can further include other non-mandatory or optional component(s).

It is also noted that the term “consisting essentially of,” as used herein refers to a composition wherein the component(s) after the term is in the presence of other known component(s) in a total amount that is less than 30% by weight of the total composition and do not contribute to or interferes with the actions or activities of the component(s).

It is also noted that the term “consisting of,” as used herein, means including, and limited to, the component(s) after the term “consisting of.” The component(s) after the term “consisting of” are therefore required or mandatory, and no other component(s) are present in the composition.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless defined otherwise herein, all technical and scientific terms used herein have the same meaning as commonly understood by one of ordinary skill in the art to which this invention pertains.

Other definitions of terms may appear throughout the specification.

### II. Variant Polypeptides Having Glucoamylase Activity

In a first aspect, the present invention relates to variant polypeptides comprising an amino acid sequence having preferably at least 60%, at least 70%, at least 75%, at least 80%, at least 85%, at least 90%, at least 92%, at least 93%, at least 94%, at least 95%, at least 96%, at least 97%, at least 98%, and even at least 99%, amino acid sequence identity to the polypeptide of SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37 having glucoamylase activity, and having at least one amino acid substitution (such as any of the substitutions shown in Table 1). In another aspect, provided herein are polypeptides comprising an amino acid sequence having at least 60%, at least 70%, at least 75%, at least 80%, at least 85%, at least 90%, at least 92%, at least 93%, at least 94%, at least 95%, at least 96%, at least 97%, at least 98%, and even at least 99%, amino acid sequence identity to a polypeptide comprising amino acid position 20-468 of SEQ ID NO:2, amino acid position 21-468 of SEQ ID NO:2, amino acid position 22-468 of SEQ ID NO:2, amino acid position 23-468 of SEQ ID NO:2, amino acid position 24-468 of SEQ ID NO:2, or amino acid position 25-468 of SEQ ID NO:2.

In some embodiments, the variant polypeptide comprises an amino acid sequence having at least 60% but less than 100% sequence identity to the polypeptide of SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37. In other embodiments, the polypeptide comprises an amino acid sequence having at least 60% but less than 100% sequence identity to the polypeptide comprising amino acid position 20-468 of SEQ ID NO:2, amino acid position 21-468 of SEQ ID NO:2, amino acid position 22-468 of SEQ ID NO:2, amino acid position 23-468 of SEQ ID NO:2, amino acid position 24-468 of SEQ ID NO:2, or amino acid position 25-468 of SEQ ID NO:2. In some embodiments, the polypeptide is non-naturally occurring (i.e., does not occur in nature and is a product of human ingenuity).

In some embodiments, the glucoamylase variant polypeptides of the present invention are homologous polypeptides comprising amino acid sequences that differ by no more than ten amino acids, no more than nine amino acids, no more than eight amino acids, no more than seven amino acids, no more than six amino acids no more than five amino acids, no more than four amino acids, no more than three amino acids, no more than two amino acids, and even no more than one amino acid from the polypeptide of SEQ ID NO:2, the polypeptide of SEQ ID NO:3, the polypeptide of SEQ ID NO:4, any one of the polypeptides of SEQ ID NOs:5-37, the polypeptide comprising amino acid position 20-468 of SEQ ID NO:2, the polypeptide comprising amino acid position 21-468 of SEQ ID NO:2, the polypeptide comprising amino acid position 22-468 of SEQ ID NO:2, the polypeptide comprising amino acid position 23-468 of SEQ ID NO:2, the polypeptide comprising amino acid position 24-468 of SEQ ID NO:2, or the polypeptide comprising amino acid position 25-468 of SEQ ID NO:2.

In some embodiments, the variant polypeptides of the present invention are the catalytic regions comprising amino acids 18 to 449 of SEQ ID NO: 4, predicted by ClustalX Hypertext Transfer Protocol Secure://world wide web.ncbi.nlm.nih.gov/pubmed/17846036.

In some embodiments, the polypeptides of the present invention have pullulan and/or panose and/or maltodextrin hydrolyzing activity.

In another aspect, the glucoamylase variants disclosed herein can, in some embodiments, comprise conservative substitution(s) of one or several amino acid residues relative to the amino acid sequence of SEQ ID NO:2, the amino acid sequence of SEQ ID NO:3, the amino acid sequence of SEQ ID NO:4, any one of the amino acid sequences of SEQ ID NOs:5-37, the polypeptide comprising amino acid position 20-468 of SEQ ID NO:2, the polypeptide comprising amino acid position 21-468 of SEQ ID NO:2, the polypeptide comprising amino acid position 22-468 of SEQ ID NO:2, the polypeptide comprising amino acid position 23-468 of SEQ ID NO:2, the polypeptide comprising amino acid position 24-468 of SEQ ID NO:2, or the polypeptide comprising amino acid position 25-468 of SEQ ID NO:2. Exemplary conservative amino acid substitutions are listed below. Some conservative substitutions (i.e., mutations) can be produced by genetic manipulation while others are produced by introducing synthetic amino acids into a polypeptide by other means.

| For Amino Acid | Code | Replace with any of |
|---|---|---|
| Alanine | A | D-Ala, Gly, beta-Ala, L-Cys, D-Cys |
| Arginine | R | D-Arg, Lys, D-Lys, homo-Arg, D-homo-Arg, Met, Ile, D-Met, D-Ile, |
| | | Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, b-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, Met, D-Met, Ile, D-Ile, |
| | | Orn, D-Orn |
| Methionine | M | D-Met, S-Me-Cys, Ile, D-Ile, Leu, D-Leu, Val, D-Val |
| Phenylalanine | F | D-Phe, Tyr, D-Thr, L-Dopa, His, D-His, Trp, D-Trp, Trans-3,4, or 5- |
| | | phenylproline, cis-3,4, |
| | | or 5-phenylproline |
| Proline | P | D-Pro, L-I-thioazolidine-4-carboxylic acid, D-or L-1-oxazolidine-4- |
| | | carboxylic acid |
| Serine | S | D-Ser, Thr, D-Thr, allo-Thr, Met, D-Met, Met(O), D-Met(O), L-Cys, D- |
| | | Cys |
| Threonine | T | D-Thr, Ser, D-Ser, allo-Thr, Met, |
| | | D-Met, Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

In some embodiments, the polypeptides of the present invention are the variants of the polypeptide of SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37, the polypeptide comprising amino acid position 20-468 of SEQ ID NO:2, the polypeptide comprising amino acid position 21-468 of SEQ ID NO:2, the polypeptide comprising amino acid position 22-468 of SEQ ID NO:2, the polypeptide comprising amino acid position 23-468 of SEQ ID NO:2, the polypeptide comprising amino acid position 24-468 of SEQ ID NO:2, or the polypeptide comprising amino acid position 25-468 of SEQ ID NO:2, or a fragment thereof having glucoamylase activity. The variant glucoamylase can comprise a deletion, substitution (such as any of the amino acid substitutions show in Table 1), insertion, or addition of one or more amino acid residues relative to the amino acid sequence of SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37 or a homologous sequence thereof. In all cases, the expression “one or more amino acid residues” refers to 10 or more, 20 or more, 30 or more, 40 or more, or 50 or more, i.e., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more amino acid residues. The amino acid substitutions (such as any of the amino acid substitutions show in Table 1), deletions and/or insertions of the polypeptide of SEQ ID NO:2, the polypeptide of SEQ ID NO:3, the polypeptide of SEQ ID NO:4, any one of the polypeptides of SEQ ID NOs:5-37, the polypeptide comprising amino acid position 20-468 of SEQ ID NO:2, the polypeptide comprising amino acid position 21-468 of SEQ ID NO:2, the polypeptide comprising amino acid position 22-468 of SEQ ID NO:2, the polypeptide comprising amino acid position 23-468 of SEQ ID NO:2, the polypeptide comprising amino acid position 24-468 of SEQ ID NO:2, or the polypeptide comprising amino acid position 25-468 of SEQ ID NO:2 can be at most 50, at most 40, at most 30, at most 20, at most 19, at most 18, at most 17 at most 16, at most 15, at most 14, at most 13, at most 12, at most 11, at most 10, at most 9, at most 8, at most 7, at most 6, at most 5, at most 4, at most 3, at most 2, and even at most 1.

In some embodiments, the variant alteration comprises or consists of a substitution at a position corresponding to position 102 of the polypeptide of SEQ ID NO: 4 or at the corresponding position of any one of SEQ ID NOs:5-37. In some embodiments, the amino acid at a position corresponding to position 102 of the polypeptide of SEQ ID NO: 4 or at the corresponding position of any one of SEQ ID NOs:5-37 is substituted with Ala, Arg, Asn, Asp, Cys, Gln, Glu, Gly, His, Leu, Ile, Lys, Met, Phe, Pro, Thr, Trp, Tyr, or Val. In some embodiments, the variant alteration comprises or consists of the substitution S102P of the polypeptide of SEQ ID NO: 4 or at the corresponding position of any one of SEQ ID NOs:5-37. In a further embodiment, the variant comprises or consists of the amino acid sequence of SEQ ID NO:2.

In some embodiments, the variant alteration comprises or consists of a substitution at a position corresponding to position 66 of the polypeptide of SEQ ID NO: 4 or at the corresponding position of any one of SEQ ID NOs:2-37. In some embodiments, the amino acid at a position corresponding to position 66 of the polypeptide of SEQ ID NO: 4 or at the corresponding position of any one of SEQ ID NOs:2-37 is substituted with Ala, Arg, Asn, Asp, Cys, Gln, Glu, Gly, His, Leu, Ile, Lys, Met, Phe, Pro, Ser, Thr, Trp, or Tyr. In some embodiments, the variant alteration comprises or consists of the substitution V66A of the polypeptide of SEQ ID NO: 4 or at the corresponding position of any one of SEQ ID NOs:2-37.

In another embodiment, the variant alteration comprises or consists of a substitution at a position corresponding to positions 66 and position 102 of the polypeptide of SEQ ID NO: 4 or at the corresponding positions of any one of SEQ ID NOs:2-37. In some embodiments, the variant alteration comprises or consists of the substitution V66A and S102P of the polypeptide of SEQ ID NO: 4 or at the corresponding positions of any one of SEQ ID NOs:2-37.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

In one embodiment, a glucoamylase variant disclosed herein having one or more amino acid substitutions can hydrolyze a disaccharide (such as, for example, maltose) to a greater extent (such as any of about a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 100%, 110%, 120%, 130%, 140%, 150% or more, inclusive of values falling in between these percentages, greater extent) compared to a parent glucoamylase lacking one or more of these substitutions. The variant glucoamylase can have one or more amino acid substitutions (such as any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 amino acid substitutions) at residue positions corresponding to positions 20, 21, 51, 66, 79, 80, 92, 102, 121, 140, 143, 157, 158, 165, 166, 192, 203, 210, 213, 214, 215, 221, 222, 233, 235, 236, 238, 243, 252, 274, 278, 281, 290, 302, 310, 321, 341, 350, 352, 354, 370, 390, 403, 404, 405, 418, 440, 441, and/or 444 of SEQ ID NO:4 and/or an equivalent position in a parent glucoamylase (such as, but not limited to, SEQ ID NO:2, SEQ ID NO:3, or any of SEQ ID NOs:5-37). The one or more substitutions at the particular positions can include one or more of X020A/P; X021S; X051L/V; X066A/C/F/M/P/T/W; X079C/T; X080K; X092M; X102G; X121G/P/M; X140I/C; X140C; X143G; X157I; X158A; X165W; X166G/R/F; X192F; X203W; X210L; X213R; X214A/E; X215D/C/F; X221R/T/M; X222M/C; X233M/Y/P; X235Y; X236A/Q; X238S/H; X243E/V; X252C/F; X274A; X278A; X281D; X290M/V; X302K/V/P/S/W; X310T/Y; X321D; X341M; X350I/T; X352D; X354L/M; X370M; X390E; X403G/R; X404K; X405Q/S/Y; X418W/E; X440G/H; X441S; and/or X444L (where X is any amino acid corresponding to the equivalent position in the parent glucoamylase) or one or more of S020A/P; K021S; A051L/V; V066A/C/F/M/P/T/W; A079C/T; G080K; V092M; P102G; T121G/P/M; M140I/C; M140C; T157I; N158A; Y165W; K166G/R/F; Y192F; D203W; R210L; D213R; N214A/E; S215D/C/F; S221R/T/M; G222M/C; S233M/Y/P; W235Y; D236A/Q; G238S/H; T243E/V; V252C/F; G274A; P278A; E281D; F290M/V; N302K/V/P/S/W; N310T/Y; L341M; K350I; T352D; V354L/M; S370M; S390E; Q403G/R; Y404K; H405Q/S/Y; R418W/E; A440G/H; Q441S; and/or A444L. Any assay known in the art can be used to determine hydrolysis of a disaccharide (such as, for example, maltose), including the assay described in the Examples section.

In a further embodiment, a glucoamylase variant disclosed herein having one or more amino acid substitutions can hydrolyze panose to a greater extent (such as any of about a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 100%, 110%, 120%, 130%, 140%, 150% or more, inclusive of values falling in between these percentages, greater extent) compared to a parent glucoamylase lacking one or more of these substitutions. Panose is a trisaccharide constituting a maltose molecule bonded to a glucose molecule by an alpha-1,6-glycosidic bond which is commonly used as a substrate to help characterise the activities of starch degrading enzymes. The variant glucoamylase can have one or more amino acid substitutions (such as any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acid substitutions) at residue positions corresponding to positions 20, 21, 23, 37, 51, 52, 67, 69, 73, 77, 79, 80, 81, 84, 92, 94, 102, 119, 121, 140, 141, 143, 164, 165, 166, 172, 210, 213, 214, 215, 218, 221, 222, 233, 236, 238, 252, 253, 274, 281, 290, 302, 321, 341, 350, 351, 352, 370, 390, 403, 404, 405, 418, 430, 440, 444, and/or 445 of SEQ ID NO:4 and/or an equivalent position in a parent glucoamylase (such as, but not limited to, SEQ ID NO:2, SEQ ID NO:3, or any of SEQ ID NOs:5-37). The one or more substitutions at the particular positions can include one or more of X020P/A; X021T/W/M; X023M/L; X037C; X051L/V; X052N; X067C; X069C/A; X073N; X077S; X079C, X080H/K; X081N; X084V/L; X092C; X094Y; X102G; X119G; X121M/L/G/P; X140C/Q/I; X141F; X143G, X164L; X165W/T; X166R; X172G; X210L; X213H; X214A/E; X215D; X218Q/A/V/Y; X221R; X222M/V/C; X233M/T/P; X236Q; X238H; X252F; X274D/K; X281D; X290M/V; X302S/K/W/Q/V; X321D; X341M/T; X350C/T; X351E; X352D/N; X370M; X390D/L/E; X403G; X403R/K; X404M/K; X405S/Q; X418E/W; X430A; X440G; X444P; and/or X445Y/M; (where X is any amino acid corresponding to the equivalent position in the parent glucoamylase) or one or more of S020P/A; K021T/W/M; E023M/L; E037C; A051L/V; G052N; S067C; V069C/A; K073N; T077S; A079C, G080H/K; D081N; 1084V/L; V092C; F094Y; X102G; S119G; T121M/L/G/P; M140C/Q/I; L141F; A143G, I164L; Y165W/T; K166R; V172G; R210L; D213H; N214A/E; S215D; S218Q/A/V/Y; S221R; G222M/V/C; S233M/T/P; D236Q; G238H; V252F; G274D/K; E281D; F290M/V; N302S/K/W/Q/V; N321D, L341M/T; K350C/T; N351E; T352D/N; S370M; S390D/L/E; Q403G; Q403R/K; Y404M/K; H405S/Q; R418E/W; T430A; A440G; A444P; and/or G445Y/M. Any assay known in the art can be used to determine hydrolysis of panose, including the assay described in the Examples section.

In another embodiment, a glucoamylase variant disclosed herein having one or more amino acid substitutions can hydrolyze pullulan to a greater extent (such as any of about a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 100%, 110%, 120%, 130%, 140%, 150% or more, inclusive of values falling in between these percentages, greater extent) compared to a parent glucoamylase lacking one or more of these substitutions. Pullulan is a polysaccharide polymer consisting of maltotriose units (also known as α-1,4-; α-1,6-glucan). Three glucose units in maltotriose are connected by an α-1,4 glycosidic bond, whereas consecutive maltotriose units are connected to each other by an α-1,6 glycosidic bond. Pullulan is commonly used as a substrate to identify glucoamylases that can hydrolyze alpha 1-6 linkages in starch. The variant glucoamylase can have one or more amino acid substitutions (such as any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 amino acid substitutions) at residue positions corresponding to positions 20, 51, 69, 73, 77, 79, 80, 81, 84, 94, 119, 121, 134, 140, 141, 143, 156, 158, 165, 166, 203, 210, 214, 215, 218, 221, 222, 233, 235, 236, 252, 253, 274, 278, 281, 290, 302, 310, 321, 341, 350, 352, 354, 370, 390, 416, 418, 434, 440, and/or 445 of SEQ ID NO:4 and/or an equivalent position in a parent glucoamylase (such as, but not limited to, SEQ ID NO:2, SEQ ID NO:3, or any of SEQ ID NOs:5-37). The one or more substitutions at the particular positions can include one or more of X020F/A/G; X051K; X069C; X073P/N; X077M/P; X079C/T; X080K; X081N; X084V/L; X094G/A; X119G/D; X121L/M; X134S; X140I/C/Q; X141F; X143G; X156P/C; X158A; X165T; X166H/A/F; X203C/Q/W/Y; X210L/M/F/N/A/I; X214A/Y/L/C; X215V/C/W/D/H; X218V/K/A/Q; X221R; X222M; X233M/P; X235Y; X236Q; X252F/H; X253K; X274K/D; X278A; X281D; X290M; X302P/S/V; X310T; X321D; X341M; X350I/T; X352D/S; X354L; X370M; X390D/L/E; X404K; X416Y; X418E/W; X434S; X440G; and/or X445M (where X is any amino acid corresponding to the equivalent position in the parent glucoamylase) or one or more of S020F/A/G; A051K; V069C; K073P/N; T077M/P; A079C/T; G080K; D081N; 1084V/L; F094G/A; S119G/D; T121L/M; E134S; M140I/C/Q; L141F; A143G; F156P/C; N158A; Y165T; K166H/A/F; D203C/Q/W/Y; R210L/M/F/N/A/I; N214A/Y/L/C; S215V/C/W/D/H; S218V/K/A/Q; S221R; G222M; S233M/P; W235Y; D236Q; V252F/H; E253K; G274K/D; P278A; E281D; F290M; N302P/S/V; N310T; N321D; L341M; K350I/T; T352D/S; V354L; S370M; S390D/L/E; Y404K; F416Y; R418E/W; A434S; A440G; and/or G445M. Any assay known in the art can be used to determine hydrolysis of pullulan, including the assay described in the Examples section.

In yet another embodiment, a glucoamylase variant disclosed herein having one or more amino acid substitutions can exhibit a higher performance index (PI) (such as any of about a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 100%, 110%, 120%, 130%, 140%, 150% or more, inclusive of values falling in between these percentages, higher PI) for the saccharification of soluble starch (i.e., improved hydrolysis of soluble starch or fragment thereof) compared to a parent glucoamylase lacking one or more of these substitutions. The variant glucoamylase can have one or more amino acid substitutions (such as any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54 amino acid substitutions) at residue positions corresponding to positions 21, 23, 37, 52, 66, 69, 73, 77, 79, 80, 81, 84, 92, 119, 121, 134, 140, 141, 157, 158, 164, 165, 210, 213, 214, 218, 221, 222, 233, 235, 236, 243, 252, 253, 274, 281, 290, 302, 341, 350, 351, 352, 370, 390, 403, 404, 405, 416, 418, 422, 440, 441, 444, and/or 445 of SEQ ID NO:4 and/or an equivalent position in a parent glucoamylase (such as, but not limited to, SEQ ID NO:2, SEQ ID NO:3, or any of SEQ ID NOs:5-37). The one or more substitutions at the particular positions can include one or more of X021T; X023M/L; X037C; X052N/F; X066A/F/T; X069C; X073N/P; X077P/M/S; X079R; X080N/H/K; X081N/S; X084L/V; X092I; X119A/G; X121M/L; X134S/Q; X140C/I/Q; X141F/K; X157A; X158A; X164L/T; X165W/T; X210W/G; X213H; X214G/Y; X218Q/F/A; X221R; X222M/V/C; X233M/P; X235F; X236Q; X243A; X252F; X253K; X274D/K/A; X281D; X290M/V; X302W/S/Q/K; X341M/T; X350C/E; X351E; X352D/N; X370M; X390D/L; X403G/R/K; X404M/K; X405S; X416C/Y; X418E/W; X422A; X440G; X441L; X444S/P; and/or X445M (where X is any amino acid corresponding to the equivalent position in the parent glucoamylase) or one or more of K021T; E023M/L; E037C; G052N/F; V066A/F/T; V069C; K073N/P; T077P/M/S; A079R; G080N/H/K; D081N/S; 1084L/V; V092I; S119A/G; T121M/L; E134S/Q; M140C/I/Q; L141F/K; T157A; N158A; 1164L/T; Y165W/T; R210W/G; D213H; N214G/Y; S218Q/F/A; S221R; G222M/V/C; S233M/P; W235F; D236Q; T243A; V252F; E253K; G274D/K/A; E281D; F290M/V; N302W/S/Q/K; L341M/T; K350C/E; N351E; T352D/N; S370M; S390D/L; Q403G/R/K; Y404M/K; H405S; F416C/Y; R418E/W; Y422A; A440G; Q441L; A444S/P; and/or G445M. PI can be determined based on the assay described in the Examples section.

In other embodiments, a glucoamylase variant disclosed herein having one or more amino acid substitutions can exhibit improved hydrolysis (such as any of about a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 100%, 110%, 120%, 130%, 140%, 150% or more, inclusive of values falling in between these percentages, improved hydrolysis) of maltodextrin compared to a parent glucoamylase lacking one or more of these substitutions. Maltodextrin consists of D-glucose units connected in chains of variable length. The glucose units are primarily linked with α(1→4) glycosidic bonds and maltodextrin is typically composed of a mixture of chains that vary from three to 17 glucose units long. Maltodextrin mimics a glucoamlyase substrate since it is generated using an alpha amylase. The variant glucoamylase can have one or more amino acid substitutions (such as any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 amino acid substitutions) at residue positions corresponding to positions 20, 21, 51, 67, 69, 73, 77, 79, 80, 81, 84, 102, 119, 121, 134, 140, 141, 143, 156, 157, 158, 165, 166, 172, 192, 203, 210, 213, 214, 215, 218, 221, 222, 233, 235, 236, 252, 274, 281, 290, 302, 310, 321, 341, 350, 351, 352, 354, 370, 390, 403, 404, 405, 418, 422, 434, 440, 444, and/or 445, of SEQ ID NO:4 and/or an equivalent position in a parent glucoamylase (such as, but not limited to, SEQ ID NO:2, SEQ ID NO:3, or any of SEQ ID NOs:5-37). The one or more substitutions at the particular positions can include one or more of X020P/A/E; X021S/M; X051V/L/K/Y; X067A; X069C; X073P/N; X077P/M; X079C/T; X080K/H; X081N; X084L/V; X102G; X119G; X121M/G/V/P; X134S/Q; X140C/I; X141F; X143G; X156P/C; X157I; X158A; X165W/T; X166G; X172G; X192F; X203W/C/M; X210L/M; X213R; X214A/E; X215D/C/F; X218V/Y/Q; X221R/T; X222M/C; X233M/P/Y; X235Y; X236Q/A; X252F; X274K/A/D; X281D; X290M/V; X302P/K/S/V/Q/W; X310T/Y; X321D; X341M; X350I/T; X351V; X352D; X354L/M; X370M; X390E/L/D; X403K/R/G; X404K/M; X405Q/S/Y; X418W/E; X422A; X434S; X440G/H; X444P; and/or X445M; (where X is any amino acid corresponding to the equivalent position in the parent glucoamylase) or one or more of S020P/A/E; K021S/M; A051V/L/K/Y; S067A; V069C; K073P/N; T077P/M; A079C/T; G080K/H; D081N; 1084L/V; P102G; S119G; T121M/G/V/P; E134S/Q; M140C/I; L141F; A143G; F156P/C; T157I; N158A; Y165W/T; K166G; V172G; Y192F; D203W/C/M; R210L/M; D213R; N214A/E; S215D/C/F; S218V/Y/Q; S221R/T; G222M/C; S233M/P/Y; W235Y; D236Q/A; V252F; G274K/A/D; E281D; F290M/V; N302P/K/S/V/Q/W; N310T/Y; N321D; L341M; K350I/T; N351V; T352D; V354L/M; S370M; S390E/L/D; Q403K/R/G; Y404K/M; H405Q/S/Y; R418W/E; Y422A; A434S; A440G/H; A444P; and/or G445M. Any assay known in the art can be used to determine hydrolysis of maltodextrin, including the assay described in the Examples section.

In still further embodiments, a glucoamylase variant disclosed herein having one or more amino acid substitutions can exhibit improved thermostability (such as any of about a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 100%, 110%, 120%, 130%, 140%, 150% or more, inclusive of values falling in between these percentages, improved thermostability) compared to a parent glucoamylase lacking one or more of these substitutions. The variant glucoamylase can have one or more amino acid substitutions (such as any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 amino acid substitutions) at residue positions corresponding to positions 23, 51, 52, 66, 67, 77, 119, 121, 134, 140, 141, 156, 157, 165, 192, 213, 221, 222, 233, 236, 252, 281, 290, 302, 350, 352, 370, 390, 403, 404, 416, and/or 422 of SEQ ID NO:4 and/or an equivalent position in a parent glucoamylase (such as, but not limited to, SEQ ID NO:2, SEQ ID NO:3, or any of SEQ ID NOs:5-37). The one or more substitutions at the particular positions can include one or more of X023M; X051Y; X052N; X066A/C/F/M/W; X067C; X077P; X119A; X121M; X134S; X140I/C; X141F; X156C; X157I; X165W/I; X192F; X213R; X221R; X222M; X233M; X236Q; X252F; X281D; X290V; X302H/Q/W/S/K; X350E/C; X352D; X370M; X390L/D; X403R; X404K/M; X416Y; and/or X422A/V (where X is any amino acid corresponding to the equivalent position in the parent glucoamylase) or one or more of E023M; A051Y; G052N; V066A/C/F/M/W; S067C; T077P; S119A; T121M; E134S; M140I/C; L141F; F156C; T157I; Y165W/I; Y192F; D213R; S221R; G222M; S233M; D236Q; V252F; E281D; F290V; N302H/Q/W/S/K; K350E/C; T352D; S370M; S390L/D; Q403R; Y404K/M; F416Y; and/or Y422A/V. Any assay known in the art can be used to determine thermostability of a polypeptide, including the assay described in the Examples section.

In another embodiment, a glucoamylase variant disclosed herein having one or more amino acid substitutions can exhibit improved decreased reversion (such as any of about a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 100%, inclusive of values falling in between these percentages, decreased reversion) compared to a parent glucoamylase lacking one or more of these substitutions. The variant glucoamylase can have one or more amino acid substitutions (such as any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 amino acid substitutions) at residue positions corresponding to positions 49, 51, 52, 66, 69, 77, 80, 94, 119, 134, 158, 164, 165, 172, 192, 213, 215, 218, 222, 233, 235, 236, 238, 243, 253, 274, 302, 338, 403, 405, 416, 418, 422, 430, 440, 441, 444, 445, and/or 449 of SEQ ID NO:4 and/or an equivalent position in a parent glucoamylase (such as, but not limited to, SEQ ID NO:2, SEQ ID NO:3, or any of SEQ ID NOs:5-37). The one or more substitutions at the particular positions can include one or more of XS049W; X051Y; X052P; X066P; X069K/C; X077P/M; X080H; X094A/G; X119D; X134W/Q; X158Y; X164T/L; X165W/I; X172G; X192R/F; X213H; X215R; X218K; X222C; X233T/P; X235F; X236A; X238H/S/N; X243P/S; X253K; X274K/A; X302H/F/P/M/Q/T; X338I; X403G; X405Q/S; X416C/Y; X416Y; X418W/E; X422A; X430A/Q; X440L/H; X441W/L; X444L/P; X445Y; and/or X449L (where X is any amino acid corresponding to the equivalent position in the parent glucoamylase) or one or more of S049W; A051Y; G052P; V066P; V069K/C; T077P/M; G080H; F094A/G; S119D; E134W/Q; N158Y; I164T/L; Y165W/I; V172G; Y192R/F; D213H; S215R; S218K; G222C; S233T/P; W235F; D236A; G238H/S/N; T243P/S; E253K; G274K/A; N302H/F/P/M/Q/T; F338I; Q403G; H405Q/S; F416C/Y; F416Y; R418W/E; Y422A; T430A/Q; A440L/H; Q441W/L; A444L/P; G445Y; and/or F449L. Any assay known in the art can be used to determine formation of reversion sugars during saccharification, including the assay described in the Examples section.

In further embodiments, a glucoamylase variant disclosed herein having one or more amino acid substitutions can exhibit improved saccharification yield (such as any of about a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 100%, 110%, 120%, 130%, 140%, 150% or more, inclusive of values falling in between these percentages, improved saccharification yield) of glucose compared to a parent glucoamylase lacking one or more of these substitutions. The variant glucoamylase can have one or more amino acid substitutions (such as any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43 amino acid substitutions) at residue positions corresponding to positions 21, 23, 51, 52, 66, 67, 79, 81, 92, 119, 140, 158, 164, 172, 192, 210, 213, 214, 215, 218, 221, 222, 233, 235, 236, 238, 243, 253, 281, 290, 302, 310, 351, 352, 354, 370, 403, 416, 422, 430, 441, and/or 445 of SEQ ID NO:4 and/or an equivalent position in a parent glucoamylase (such as, but not limited to, SEQ ID NO:2, SEQ ID NO:3, or any of SEQ ID NOs:5-37). The one or more substitutions at the particular positions can include one or more of X021S/W; X023L; X051Y/K; X052N; X066A/C/F/M/W; X067M/C/A; X079R; X081S; X092C/M; X119A; X140I; X158A/Y; X164L; X172L/G; X192F; X210W; X213H; X214Y; X215F/C/R; X218H/K/A; X221M/T; X222V; X233T; X235Y/F; X236S/A; X238T/V/M; X243A; X253K; X281P/D; X290V; X302K/Q/H/W; X310V/T; X351V; X352S; X354L; X370R; X403K; X416Y; X422F; X430A; X441L; and/or X445Y (where X is any amino acid corresponding to the equivalent position in the parent glucoamylase) or one or more of K021S/W; E023L; A051Y/K; G052N; V066A/C/F/M/W; S067M/C/A; A079R; D081S; V092C/M; S119A; M140I; N158A/Y; I164L; V172L/G; Y192F; R210W; D213H; N214Y; S215F/C/R; S218H/K/A; S221M/T; G222V; S233T; W235Y/F; D236S/A; G238T/V/M; T243A; E253K; E281P/D; F290V; N302K/Q/H/W; N310V/T; N351V; T352S; V354L; S370R; Q403K; F416Y; Y422F; T430A; Q441L; and/or G445Y. Any assay known in the art can be used to determine saccharification yield, including the assay described in the Examples section.

Accordingly, in still further embodiments, a glucoamylase variant disclosed herein having one or more amino acid substitutions can exhibit improvements (such as any of about a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 55%, 60%, 65%, 70%, 75%, 80%, 85%, 90%, 95%, 100%, 110%, 120%, 130%, 140%, 150% or more, inclusive of values falling in between these percentages, improvements of two or more of (such as 2, 3, 4, 5, 6, 7, or 8) i) improved hydrolysis of a disaccharide (such as, for example, maltose); ii) greater hydrolysis of panose; iii) greater hydrolysis of pullulan; iv) a higher PI for the saccharification of soluble starch (i.e., improved hydrolysis of soluble starch or a fragment thereof); v) improved hydrolysis of maltodextrin; vi) improved thermostability; vii) less reversion to saccharides having a DP greater than or equal to 2; and/or viii) improved saccharification yield of glucose compared to a parent glucoamylase lacking one or more of these substitutions. The variant glucoamylase can have one or more amino acid substitutions (such as any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or 71 amino acid substitutions) at residue positions corresponding to positions 20, 21, 23, 37, 51, 52, 66, 67, 69, 73, 77, 79, 80, 81, 84, 92, 94, 102, 119, 121, 134, 140, 141, 156, 157, 158, 164, 165, 166, 172, 192, 203, 210, 213, 214, 215, 218, 221, 222, 233, 235, 236, 238, 243, 252, 253, 274, 278, 281, 290, 302, 310, 341, 350, 351, 352, 354, 370, 390, 403, 404, 405, 416, 418, 422, 430, 440, 441, 444, 445, and/or 449 of SEQ ID NO:4 and/or an equivalent position in a parent glucoamylase (such as, but not limited to, SEQ ID NO:2, SEQ ID NO:3, or any of SEQ ID NOs:5-37). The one or more substitutions at the particular positions can include one or more of X020A/P; X021M/S/T/W; X023L/M; X037C; X051K/L/V/Y; X052N; X066A/C/F/M/W; X067A/C; X069C; X073N/P; X077M/P/S; X079R/T; X080H/K; X081N/S; X084L/V; X092C/M; X094A/G; X102G; X119A/D/G; X121G/L/M/P; X134Q/S; X140C/I/Q; X141F; X156C/P; X157I; X158A/Y; X164L/T; X165I/T/W; X166G/F/R; X172G; X192F; X203C/W; X210L/M/W; X213H/R; X214A/E/Y; X215C/D/F/R; X218A/K/Q/V/Y; X221M/R/T; X222C/M/V; X233M/P/T/Y; X235F/Y; X236A/Q; X238H/S; X243A; X252F; X253K; X274A/D/K; X278A; X281D; X290M/V; X290V; X302H/K/P/Q/S/V/W; X310T/Y; X341M/T; X350C/E/I; X351E/V; X352D/N/S; X354L/M; X370M; X390D/E/L; X403G/K/R; X404I/K/M; X405Q/S/Y; X416C/Y; X418E/W; X422A; X430A; X440G/H; X441L; X444L/P; and/or X445M/Y; (where X is any amino acid corresponding to the equivalent position in the parent glucoamylase) or one or more of S020A/P; K021M/S/T/W; E023L/M; E037C; A051K/L/V/Y; G052N; V066A/C/F/M/W; S067A/C; V069C; K073N/P; T077M/P/S; A079R/T; G080H/K; D081N/S; 1084L/V; V092C/M; F094A/G; P102G; S119A/D/G; T121G/L/M/P; E134Q/S; M140C/I/Q; L141F; F156C/P; T157I; N158A/Y; 1164L/T; Y165I/T/W; K166G/F/R; V172G; Y192F; D203C/W; R210L/M/W; D213H/R; N214A/E/Y; S215C/D/F/R; S218A/K/Q/V/Y; S221M/R/T; G222C/M/V; S233M/P/T/Y; W235F/Y; D236A/Q; G238H/S; T243A; V252F; E253K; G274A/D/K; P278A; E281D; F290M/V; F290V; N302H/K/P/Q/S/V/W; N310T/Y; L341M/T; K350C/E/I; N351E/V; T352D/N/S; V354L/M; S370M; S390D/E/L; Q403G/K/R; Y404I/K/M; H405Q/S/Y; F416C/Y; R418E/W; Y422A; T430A; A440G/H; Q441L; A444L/P; and/or G445M/Y.

Multiple amino acid substitutions (such as any of the substitutions shown in Table 1), deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochem. 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Further provided herein, in additional embodiments, are glucoamylase variants having two or more substitutions at residue positions corresponding to a) X236S and X281D; b) X215R and X441W; c) X321D and X434S; d) X143G and X434S; e) X079C and X143G; f) X350T and X434S; g) X351E and X403K; h) X052N and X084L; i) X243P and X290V; j) X290V and X350E; k) X302K and X441W; 1) X156C and X404K; m) X067M and X404K; n) X052N and X141F; o) X052N and X351E; p) X233M and X445Y; q) X066C and X233M; r) X218H and X290V; s) X067M and X302H; t) X066C and X119A; u) X243P and X445Y; v) X192F and X243P; w) X156C and X243P; x) X023L and X066C; y) X023L-X119A; z) X020E and X192F; aa) X192F and X310V; bb) X023M and X302H; cc) X192F and X416Y; dd) X119A and X302H; ee) X235Y and X416Y; ff) X052N and X404K; gg) X023M and X449Y; hh) X158A and X172L; ii) X172L and X290V; jj) X023M and X210L; kk) X210L and X449Y; 11) X157I and X281D; mm) X164T and X215R; nn) X140C and X422V; oo) X119A and X302K; or pp) X052N and X416Y, where X is any amino acid corresponding to the equivalent position in SEQ ID NO:4 and/or an equivalent position in a parent glucoamylase (such as, but not limited to, SEQ ID NO:2, SEQ ID NO:3, or any of SEQ ID NOs:5-37). In other embodiments, the two or more amino acid substitutions can be a) D236S and E281D; b) S215R and Q441W; c) N321D and A434S; d) A143G and A434S; e) A079C and A143G; f) K350T and A434S; g) N351E and Q403K; h) G052N and I084L; i) T243P and F290V; j) F290V and K350E; k) N302K and Q441W; 1) F156C and Y404K; m) S067M and Y404K; n) G052N and L141F; o) G052N and N351E; p) S233M and G445Y; q) A066C and S233M; r) S218H and F290V; s) S067M and N302H; t) A066C and S119A; u) T243P and G445Y; v) Y192F and T243P; w) F156C and T243P; x) E023L and A066C; y) E023L and S119A; z) S020E and Y192F; aa) Y192F and N310V; bb) E023M and N302H; cc) Y192F and F416Y; dd) S119A and N302H; ee) W235Y and F416Y; ff) G052N and Y404K; gg) E023M and F449Y; hh) N158A and V172L; ii) V172L and F290V; jj) E023M and R210L; kk) R210L and F449Y; 11) T157I and E281D; mm) I164T and S215R; nn) M140C and Y422V; oo) S119A and N302K; or pp) G052N and F416Y. The variant glucoamylase exhibits one or more of i) improved hydrolysis of maltose; ii) improved hydrolysis of panose; iii) improved hydrolysis of pullulan; iv) a higher PI for the saccharification of soluble starch (i.e. improved hydrolysis of soluble starch or a fragment thereof); v) improved hydrolysis of maltodextrin; vi) improved thermostability; vii) less reversion to saccharides having a DP greater than or equal to 2; and/or viii) improved saccharification yield of glucose compared to a parent glucoamylase lacking two or more of these substitutions.

Also provided herein, in additional embodiments, are glucoamylase variants having three or more substitutions at residue positions corresponding to a) X141F, X281D, and X441W; b) X143G, X321D, and X434S; c) X321D, X350T, and X434S; d) X067M, X158A, and X281D; e) X156C, X192F, and X403K; f) X052N, X140C, and X422V; g) X066C, X119A, and X164T; h) X066C, X233M, and X445Y; i) X156C, X192F, and X243P; j) X023L, X066C, and X119A; k) X023M, X119A, and X404K; 1) X310V, X416Y, and X445Y; m) X158A, X221R, and X290V; n) X023M, X052N, and X404K; o) X081S, X157I, and X236S; p) X243P, X302K, and X416Y; q) X140C, X302K, and X422V; or r) X052N, X416Y, and X445Y, where X is any amino acid corresponding to the equivalent position in SEQ ID NO:4 and/or an equivalent position in a parent glucoamylase (such as, but not limited to, SEQ ID NO:2, SEQ ID NO:3, or any of SEQ ID NOs:5-37). In other embodiments, the three or more amino acid substitutions can be a) L141F, E281D, and Q441W; b) A143G, N321D, and A434S; c) N321D, K350T, and A434S; d) S067M, N158A, and E281D; e) F156C, Y192F, and Q403K; f) G052N, M140C, and Y422V; g) A066C, S119A, and I164T; h) A066C, S233M, and G445Y; i) F156C, Y192F, and T243P; j) E023L, A066C, and S119A; k) E023M, S119A, and Y404K; 1) N310V, F416Y, and G445Y; m) N158A, S221R, and F290V; n) E023M, G052N, and Y404K; o) D081S, T157I, and D236S; p) T243P, N302K, and F416Y; q) M140C, N302K, and Y422V; or r) G052N, F416Y, and G445Y. The variant glucoamylase exhibits one or more of i) improved hydrolysis of maltose; ii) improved hydrolysis of panose; iii) improved hydrolysis of pullulan; iv) a higher PI for the saccharification of soluble starch (i.e. improved hydrolysis of soluble starch or a fragment thereof); v) improved hydrolysis of maltodextrin; vi) improved thermostability; vii) less reversion to saccharides having a DP greater than or equal to 2; and/or viii) improved saccharification yield of glucose compared to a parent glucoamylase lacking three or more of these substitutions.

Additionally provided herein, in additional embodiments, are glucoamylase variants having four or more substitutions at residue positions corresponding to a) X215R, X236S, X281D, and X441W; b) X052N, X084L, X140C, and X422V; c) X020E, X156C, X192F, and X243P; d) X023M, X221R, and X404K; e) X158A, X172L, X221R, and X290V; f) X140C, X165W, X302K, and X422V; or g) X119A, X253F, X310V, and X403K, where X is any amino acid corresponding to the equivalent position in SEQ ID NO:4 and/or an equivalent position in a parent glucoamylase (such as, but not limited to, SEQ ID NO:2, SEQ ID NO:3, or any of SEQ ID NOs:5-37). In other embodiments, the four or more amino acid substitutions can be a) S215R, D236S, E281D, and Q441W; b) G052N, I084L, M140C, and Y422V; c) S020E, F156C, Y192F, and T243P; d) E023M, S119A, S221R, and Y404K; e) N158A, V172L, S221R, and F290V; f) M140C, Y165W, N302K, and Y422V; or g) S119A, E253F, N310V, and Q403K. The variant glucoamylase exhibits one or more of i) improved hydrolysis of maltose; ii) improved hydrolysis of panose; iii) improved hydrolysis of pullulan; iv) a higher PI for the saccharification of soluble starch (i.e. improved hydrolysis of soluble starch or a fragment thereof); v) improved hydrolysis of maltodextrin; vi) improved thermostability; vii) less reversion to saccharides having a DP greater than or equal to 2; and/or viii) improved saccharification yield of glucose compared to a parent glucoamylase lacking four or more of these substitutions.

Still further provided herein, in additional embodiments, are glucoamylase variants having five or more substitutions at residue positions corresponding to X067M, X157I, X218H, X302H, and X416Y, where X is any amino acid corresponding to the equivalent position in SEQ ID NO:4 and/or an equivalent position in a parent glucoamylase or fragment thereof (such as, but not limited to, SEQ ID NO:2, SEQ ID NO:3, or any of SEQ ID NOs:5-37). In other embodiments, the four or more amino acid substitutions can be S067M, T157I, S218H, N302H, and F416Y. The variant glucoamylase exhibits one or more of i) improved hydrolysis of maltose; ii) improved hydrolysis of panose; iii) improved hydrolysis of pullulan; iv) a higher PI for the saccharification of soluble starch (i.e. improved hydrolysis of soluble starch or a fragment thereof); v) improved hydrolysis of maltodextrin; vi) improved thermostability; vii) less reversion to saccharides having a DP greater than or equal to 2; and/or viii) improved saccharification yield of glucose compared to a parent glucoamylase lacking five or more of these substitutions.

In further embodiments, one or more (such as 1, 2, 3, 4, 5, 6, 7, 8, 9, or 10 or more) consensus N-linked glycosylation sites (i.e. an NXS/T consensus N-linked glycosylation site, where X can be any amino acid except proline and the N (Asp) is glycosylated) can be introduced into any of the variant glucoamylases disclosed herein or into the glucoamylase of SEQ ID NO:4) to improve one or more property of the glucoamylase such as, without limitation, of i) improved hydrolysis of maltose; ii) improved hydrolysis of panose; iii) improved hydrolysis of pullulan; iv) a higher PI for the saccharification of soluble starch (i.e. improved hydrolysis of soluble starch or a fragment thereof); v) improved hydrolysis of maltodextrin; vi) improved thermostability; vii) less reversion to saccharides having a DP greater than or equal to 2; and/or viii) improved saccharification yield of glucose compared to a parent glucoamylase lacking one or more additional glycosylation sites. Amino acid substitutions to introduce glycosylation sites into the variant glucoamylase polypeptides can be made at residue positions corresponding to positions 81, 83, 153, 370 or 372 of SEQ ID NO:4 and/or an equivalent position in a parent glucoamylase. In some embodiments, said one or more substitutions comprise one or more substitutions at position D81N, K83T, A153T, S370N, and/or A372S.

As shown in Example 7, a substitution at the Asn glycosylation site at position 75 was observed to lead to a drastic reduction in catalytic activity. Accordingly, in some embodiments, the variant glucoamylases disclosed herein have an N-linked glycosylation at position N075 of SEQ ID NO:4 and/or an equivalent position in a parent glucoamylase (such as, but not limited to, SEQ ID NO:2, SEQ ID NO:3, or any of SEQ ID NOs:5-37).

### III. Production of Glucoamylase

The variant glucoamylases disclosed herein can be produced in host cells, for example, by secretion or intracellular expression. A cultured cell material (e.g., a whole-cell broth) comprising a glucoamylase can be obtained following secretion of the glucoamylase into the cell medium. Optionally, the glucoamylase can be isolated from the host cells, or even isolated from the cell broth, depending on the desired purity of the final glucoamylase. A gene encoding a glucoamylase can be cloned and expressed according to methods well known in the art. Suitable host cells include bacterial, fungal (including yeast and filamentous fungi), and plant cells (including algae). Particularly useful host cells include Aspergillus spp. (such as, without limitation, Aspergillus niger or Aspergillus oryzae) a Trichoderma spp. (such as, Trichoderma reesi) or a Myceliopthora spp. (such as Myceliopthora thermophila). Other host cells include bacterial cells, e.g., Bacillus spp. (such as, Bacillus subtilis or B. licheniformis), as well as Streptomyces spp. A suitable yeast host organism can be selected from Schizosaccharomyces species or a species of Saccharomyces, including Saccharomyces cerevisiae or a species belonging to Schizosaccharomyces such as, for example, S. pombe species. A strain of the methylotrophic yeast species, Pichia pastoris, can be used as the host organism.

Additionally, the host may express one or more accessory enzymes, proteins, peptides. These may benefit liquefaction, saccharification, fermentation, SSF, and downstream processes. Furthermore, the host cell may produce ethanol and other biochemicals or biomaterials in addition to enzymes used to digest the various feedstock(s). Such host cells may be useful for fermentation or simultaneous saccharification and fermentation processes to reduce or eliminate the need to add enzymes.

### A. Vectors

A DNA construct comprising a nucleic acid encoding a variant glucoamylase polypeptide disclosed herein can be constructed such that it is suitable to be expressed in a host cell. Because of the known degeneracy in the genetic code, different polynucleotides that encode an identical amino acid sequence can be designed and made with routine skill. It is also known that, depending on the desired host cells, codon optimization may be required prior to attempting expression.

A polynucleotide encoding a variant glucoamylase polypeptide of the present disclosure can be incorporated into a vector. Vectors can be transferred to a host cell using known transformation techniques, such as those disclosed below.

A suitable vector may be one that can be transformed into and/or replicated within a host cell. For example, a vector comprising a nucleic acid encoding a variant glucoamylase polypeptide disclosed herein can be transformed and/or replicated in a bacterial host cell as a means of propagating and amplifying the vector. The vector may also be suitably transformed into an expression host, such that the encoding polynucleotide is expressed as a functional glucoamylase enzyme.

A representative useful vector is pTrex3gM (see, Published US Patent Application 20130323798) and pTTT (see, Published US Patent Application 20110020899), which can be inserted into genome of host. The vectors pTrex3gM and pTTT can both be modified with routine skill such that they comprise and express a polynucleotide encoding a variant glucoamylase polypeptide of the invention.

An expression vector normally comprises control nucleotide sequences such as a promoter, operator, ribosome binding site, translation initiation signal and optionally, a repressor gene or one or more activator genes. Additionally, the expression vector may comprise a sequence coding for an amino acid sequence capable of targeting the glucoamylase to a host cell organelle such as a peroxisome, or to a particular host cell compartment. For expression under the direction of control sequences, the nucleic acid sequence of the variant glucoamylase is operably linked to the control sequences in proper manner with respect to expression.

A polynucleotide encoding a variant glucoamylase polypeptide disclosed herein can be operably linked to a promoter, which allows transcription in the host cell. The promoter may be any DNA sequence that shows transcriptional activity in the host cell of choice and may be derived from genes encoding proteins either homologous or heterologous to the host cell. Examples of promoters for directing the transcription of the DNA sequence encoding a glucoamylase, especially in a bacterial host, include the promoter of the lac operon of E. coli, the Streptomyces coelicolor agarase gene dagA or celA promoters, the promoters of the Bacillus licheniformis amylase gene (amyL), the promoters of the Bacillus stearothermophilus maltogenic amylase gene (amyM), the promoters of the Bacillus amyloliquefaciens amylase (amyQ), the promoters of the Bacillus subtilis xylA and xylB genes, and the like.

For transcription in a fungal host, examples of useful promoters include those derived from the gene encoding Aspergillus oryzae TAKA amylase, Rhizomucor miehei aspartic proteinase, Aspergillus niger neutral α-amylase, Aspergillus niger acid stable α-amylase, Aspergillus niger glucoamylase, Rhizomucor miehei lipase, Aspergillus oryzae alkaline protease, Aspergillus oryzae triose phosphate isomerase, Aspergillus nidulans acetamidase and the like. When a gene encoding a glucoamylase is expressed in a bacterial species such as an E. coli, a suitable promoter can be selected, for example, from a bacteriophage promoter including a T7 promoter and a phage lambda promoter. Along these lines, examples of suitable promoters for the expression in a yeast species include but are not limited to the Gal 1 and Gal 10 promoters of Saccharomyces cerevisiae and the Pichia pastoris AOX1 or AOX2 promoters. Expression in filamentous fungal host cells often involves cbh1, which is an endogenous, inducible promoter from T. reesei. See Liu et al. (2008) Acta Biochim. Biophys. Sin (Shanghai) 40(2): 158-65.

The coding sequence can be operably linked to a signal sequence. The DNA encoding the signal sequence may be a DNA sequence naturally associated with the variant glucoamylase gene of interest to be expressed, or may be from a different genus or species as the variant glucoamylase (i.e. the species from which the variant was derived). A signal sequence and a promoter sequence comprising a DNA construct or vector can be introduced into a fungal host cell and can be derived from the same source. For example, the signal sequence may be the Trichoderma reesei cbh1 signal sequence, which is operably linked to a cbh1 promoter.

An expression vector may also comprise a suitable transcription terminator and, in eukaryotes, polyadenylation sequences operably linked to the DNA sequence encoding a glucoamylase. Termination and polyadenylation sequences may suitably be derived from the same sources as the promoter.

The vector may also comprise a selectable marker, e.g., a gene the product of which complements a defect in the isolated host cell, such as the dal genes from B. subtilis or B. licheniformis, or a gene that confers antibiotic resistance such as, e.g., ampicillin, kanamycin, chloramphenicol or tetracycline resistance. Furthermore, the vector may comprise Aspergillus selection markers such as amdS, argB, niaD and xxsC, a marker giving rise to hygromycin resistance, or the selection may be accomplished by co-transformation, such as known in the art. See e.g., Published International PCT Application WO 91/17243.

### B. Transformation and Culture of Host Cells

An isolated cell, either comprising a DNA construct or an expression vector, is advantageously used as a host cell in the recombinant production of a variant glucoamylase. The cell may be transformed with the DNA construct encoding the enzyme, conveniently by integrating the DNA construct (in one or more copies) in the host chromosome. This integration is generally considered to be an advantage, as the DNA sequence is more likely to be stably maintained in the cell. Integration of the DNA constructs into the host chromosome may be performed according to conventional methods, e.g., by homologous or heterologous recombination. Alternatively, the cell may be transformed with an expression vector in connection with the different types of host cells.

Examples of suitable bacterial host organisms are Gram positive bacterial species such as Bacillaceae including Bacillus subtilis, Bacillus licheniformis, Bacillus lentus, Bacillus brevis, Geobacillus (formerly Bacillus) stearothermophilus, Bacillus alkalophilus, Bacillus amyloliquefaciens, Bacillus coagulans, Bacillus lautus, Bacillus megaterium, and Bacillus thuringiensis; Streptomyces species such as Streptomyces murinus; lactic acid bacterial species including Lactococcus sp. such as Lactococcus lactis; Lactobacillus sp. including Lactobacillus reuteri; Leuconostoc sp.; Pediococcus sp.; and Streptococcus sp. Alternatively, strains of a Gram negative bacterial species belonging to Enterobacteriaceae including E. coli, or to Pseudomonadaceae can be selected as the host organism.

A suitable yeast host organism can be selected from the biotechnologically relevant yeasts species such as but not limited to yeast species such as Pichia sp., Hansenula sp., or Kluyveromyces, Yarrowinia, Schizosaccharomyces species or a species of Saccharomyces, including Saccharomyces cerevisiae or a species belonging to Schizosaccharomyces such as, for example, S. pombe species. A strain of the methylotrophic yeast species, Pichia pastoris, can be used as the host organism. Alternatively, the host organism can be a Hansenula species.

Suitable host organisms among filamentous fungi include species of Aspergillus, e.g., Aspergillus niger, Aspergillus oryzae, Aspergillus tubigensis, Aspergillus awamori, or Aspergillus nidulans. Alternatively, strains of a Fusarium species, e.g., Fusarium oxysporum or of a Rhizomucor species such as Rhizomucor miehei can be used as the host organism. Other suitable strains include Thermomyces and Mucor species. In addition, Trichoderma sp. can be used as a host. A glucoamylase expressed by a fungal host cell can be glycosylated, i.e., will comprise a glycosyl moiety. The glycosylation pattern can be the same or different as present in the wild-type glucoamylase. The type and/or degree of glycosylation may impart changes in enzymatic and/or biochemical properties.

It is advantageous to delete genes from expression hosts, where the gene deficiency can be cured by the transformed expression vector. Known methods may be used to obtain a fungal host cell having one or more inactivated genes. In one non-limiting embodiment, any gene from a Trichoderma sp. or other filamentous fungal host that has been cloned can be deleted, for example, cbh1, cbh2, egl1, and egl2 genes. Gene deletion may be accomplished by inserting a form of the desired gene to be inactivated into a plasmid by methods known in the art.

General transformation techniques are known in the art. See, e.g., Sambrook et al. (2001), supra. The expression of heterologous protein in Trichoderma is described, for example, in U.S. Pat. No. 6,022,725. Reference is also made to Cao et al. (2000) Science 9:991-1001 for transformation of Aspergillus strains. Genetically stable transformants can be constructed with vector systems whereby the nucleic acid encoding a glucoamylase is stably integrated into a host cell chromosome. Transformants are then selected and purified by known techniques. The deletion plasmid is then cut at an appropriate restriction enzyme site(s), internal to the desired gene coding region, and the gene coding sequence or part thereof is replaced with a selectable marker. Flanking DNA sequences from the locus of the gene to be deleted (preferably between about 0.5 to 2.0 kb) remain on either side of the marker gene. An appropriate deletion plasmid will generally have unique restriction enzyme sites present therein to enable the fragment containing the deleted gene, including the flanking DNA sequences and the selectable markers gene to be removed as a single linear piece.

Depending upon the host cell used post-transcriptional and/or post-translational modifications may be made. One non-limiting example of a post-transcriptional and/or post-translational modification is “clipping” or “truncation” of a polypeptide. In another instance, this clipping may result in taking a mature glucoamylase polypeptide and further removing N or C-terminal amino acids (such as about 1, 2, 3, 4, 5, 6, 7, 8, 9, or 10 or more N or C-terminal amino acids) to generate truncated forms of the glucoamylase that retain enzymatic activity.

Other examples of post-transcriptional or post-translational modifications include, but are not limited to, myristoylation, glycosylation, truncation, lipidation and tyrosine, serine or threonine phosphorylation. The skilled person will appreciate that the type of post-transcriptional or post-translational modifications that a protein may undergo may depend on the host organism in which the protein is expressed.

Further sequence modifications of polypeptides post expression may occur. This includes, but is not limited to, oxidation, deglycosylation, glycation, etc. It is known that glycation can affect the activity of glucoamylase when subjected to incubation with glucose or other reducing sugars especially at temperatures above 30° C. and neutral or alkaline pH. Protein engineering to eliminate lysine residues can be used to prevent such modification. An example of this can be found in U.S. Pat. No. 8,507,240. For example, yeast expression can result in highly glycosylated polypeptides resulting in an apparent increased molecular weight. Also, WO2013/119470 (incorporated by reference herein) having international publication date Aug. 15, 2013 relates to phytases having increased stability believed to be due to increased glycosylation.

### C. Expression and Fermentation

A method of producing any of the variant glucoamylases disclosed herein may comprise cultivating a host cell under conditions conducive to the production of the enzyme and recovering the enzyme from the cells and/or culture medium.

The medium used to cultivate the cells may be any conventional medium suitable for growing the host cell and obtaining expression of a variant glucoamylase polypeptide. Suitable media and media components are available from commercial suppliers or may be prepared according to published recipes (e.g., as described in catalogues of the American Type Culture Collection).

Any of the fermentation methods well known in the art can suitably used to ferment the transformed or the derivative fungal strain as described above. In some embodiments, fungal cells are grown under batch or continuous fermentation conditions.

### D. Methods for Enriching and Purification

Separation and concentration techniques are known in the art and conventional methods can be used to prepare a concentrated solution or broth comprising a variant glucoamylase polypeptide of the invention.

After fermentation, a fermentation broth is obtained, the microbial cells and various suspended solids, including residual raw fermentation materials, are removed by conventional separation techniques in order to obtain a glucoamylase solution. Filtration, centrifugation, microfiltration, rotary vacuum drum filtration, ultrafiltration, centrifugation followed by ultrafiltration, extraction, or chromatography, or the like, are generally used.

It may at times be desirable to concentrate a solution or broth comprising a glucoamylase polypeptide to optimize recovery. Use of un-concentrated solutions or broth would typically increase incubation time in order to collect the enriched or purified enzyme precipitate.

### IV. Compositions

The present invention also relates to compositions comprising a polypeptide (such as a glucoamylase, for example, any of the variant glucoamylases disclosed herein) and/or a starch substrate. In some embodiments, a variant glucoamylases comprising an amino acid sequence that is at least about 80%, at least about 85%, at least about 90%, at least about 95%, at least about 96%, at least about 97%, at least about 98%, at least about 99%, identical to that of SEQ ID NO: 4 (or any of SEQ ID NOs:2-30) can also be used in the enzyme composition. Preferably, the compositions are formulated to provide desirable characteristics such as low color, low odor and acceptable storage stability at a temperature of about 4-40° C. and a pH of about 3-7.

The composition may comprise a variant glucoamylase polypeptide of the present invention as the major enzymatic component. Alternatively, the composition may comprise multiple enzymatic activities, such as an aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, alpha-glucosidase, beta-glucosidase, beta-amylase, isoamylase, haloperoxidase, invertase, laccase, lipase, mannosidase, oxidase, pectinolytic enzyme, peptidoglutaminase, peroxidase, phytase, polyphenoloxidase, proteolytic enzyme, pullulanase, ribonuclease, transglutaminase, xylanase or a combination thereof, which may be added in effective amounts well known to the person skilled in the art.

The polypeptide compositions may be prepared in accordance with methods known in the art and may be in the form of a liquid or a dry composition. For instance, the compositions comprising the present variant glucoamylases may be aqueous or non-aqueous formulations, granules, powders, gels, slurries, pastes, etc., which may further comprise any one or more of the additional enzymes listed, herein, along with buffers, salts, preservatives, water, co-solvents, surfactants, and the like. Such compositions may work in combination with endogenous enzymes or other ingredients already present in a slurry, water bath, washing machine, food or drink product, etc., for example, endogenous plant (including algal) enzymes, residual enzymes from a prior processing step, and the like. The polypeptide to be included in the composition may be stabilized in accordance with methods known in the art.

The composition may be cells expressing the polypeptide, including cells capable of producing a product from fermentation. Such cells may be provided in a liquid or in dry form along with suitable stabilizers. Such cells may further express additional polypeptides, such as those mentioned, above.

The dosage of the polypeptide composition of the invention and other conditions under which the composition is used may be determined on the basis of methods known in the art.

The above composition is suitable for use in liquefaction, saccharification, and/or fermentation process, preferably in starch conversion, especially for producing syrup and fermentation products, such as ethanol. The composition is also suitable for use in animal nutrition (such as a component of an animal feed) and fermented beverage production.

### V. Uses and Methods

The present invention is also directed to use of a variant glucoamylase polypeptide or composition of the present invention in a liquefaction, a saccharification and/or a fermentation process. The variant glucoamylase polypeptide or composition may be used in a single process, for example, in a liquefaction process, a saccharification process, or a fermentation process. The variant glucoamylase polypeptide or composition may also be used in a combination of processes for example in a liquefaction and saccharification process, in a liquefaction and fermentation process, or in a saccharification and fermentation process, preferably in relation to starch conversion.

### A. Saccharification

The liquefied starch may be saccharified into a syrup rich in lower DP (e.g., DP1+DP2) saccharides, using alpha-amylases and variant glucoamylases, optionally in the presence of another enzyme(s). The exact composition of the products of saccharification depends on the combination of enzymes used, as well as the type of starch processed. Advantageously, the syrup obtainable using the provided variant glucoamylase may contain a weight percent of DP1 of the total oligosaccharides in the saccharified starch exceeding 90%, e.g., 90%-98% or 95%-97%. The weight percent of DP2 in the saccharified starch may be as low as possible, about less than 3%, e.g., 0-3% or 0-2.8%.

Whereas liquefaction is generally run as a continuous process, saccharification is often conducted as a batch process. Saccharification conditions are dependent upon the nature of the Liquefact and type of enzymes available. In some cases, a saccharification process may involve temperatures of about 60-65° C. and a pH of about 4.0-4.5, e.g., pH 4.3. Saccharification may be performed, for example, at a temperature between about 40° C., about 50° C., or about 55° C. to about 60° C. or about 65° C., necessitating cooling of the Liquefact. The pH may also be adjusted as needed. Saccharification is normally conducted in stirred tanks, which may take several hours to fill or empty. Enzymes typically are added either at a fixed ratio to dried solids, as the tanks are filled, or added as a single dose at the commencement of the filling stage. A saccharification reaction to make a syrup typically is run over about 24-72 hours, for example, 24-48 hours. A pre-saccharification can be added before saccharification in a simultaneous saccharification and fermentation (SSF), for typically 40-90 minutes at a temperature between 30-65° C., typically about 60° C.

### B. Raw Starch Hydrolysis

The present invention provides a use of the variant glucoamylase of the invention for producing glucoses and the like from raw starch or granular starch. Generally, glucoamylase of the present invention either alone or in the presence of an alpha-amylase can be used in raw starch hydrolysis (RSH) or granular starch hydrolysis (GSH) process for producing desired sugars and fermentation products. The granular starch is solubilized by enzymatic hydrolysis below the gelatinization temperature. Such “low-temperature” systems (known also as “no-cook” or “cold-cook”) have been reported to be able to process higher concentrations of dry solids than conventional systems (e.g., up to 45%).

A “raw starch hydrolysis” process (RSH) differs from conventional starch treatment processes, including sequentially or simultaneously saccharifying and fermenting granular starch at or below the gelatinization temperature of the starch substrate typically in the presence of at least a glucoamylase and/or amylase.

The variant glucoamylase of the invention may also be used in combination with an enzyme that hydrolyzes only alpha-(1, 6)-glucosidic bonds in molecules comprising at least four glucosyl residues. Preferably, the variant glucoamylase of the invention is used in combination with pullulanase or isoamylase. The use of isoamylase and pullulanase for debranching of starch, the molecular properties of the enzymes, and the potential use of the enzymes together with glucoamylase is described in G. M. A. van Beynum et al., Starch Conversion Technology, Marcel Dekker, New York, 1985, 101-142.

### C. Fermentation

The soluble starch hydrolysate, particularly a glucose rich syrup, can be fermented by contacting the starch hydrolysate with a fermenting organism typically at a temperature around 32° C., such as from 30° C. to 35° C. “Fermenting organism” refers to any organism, including bacterial and fungal organisms, suitable for use in a fermentation process and capable of producing desired a fermentation product. Especially suitable fermenting organisms are able to ferment, i.e., convert, sugars, such as glucose or maltose, directly or indirectly into the desired fermentation product. Examples of fermenting organisms include yeast, such as Saccharomyces cerevisiae and bacteria, e.g., Zymomonas mobilis, expressing alcohol dehydrogenase and pyruvate decarboxylase. The ethanologenic microorganism can express xylose reductase and xylitol dehydrogenase, which convert xylose to xylulose. Improved strains of ethanologenic microorganisms, which can withstand higher temperatures, for example, are known in the art and can be used. See Liu et al. (2011) Sheng Wu Gong Cheng Xue Bao 27:1049-56. Yeast that can be used for alcohol production include, but are not limited to, Saccharomyces spp., including S. cerevisiae, as well as Kluyveromyces, Lachancea and Schizosaccharomyces spp. Numerous yeast strains are commercially available, many of which have been selected or genetically engineered for desired characteristics, such as high alcohol production, rapid growth rate, and the like. The temperature and pH of the fermentation will depend upon the fermenting organism. Microorganisms that produce other metabolites, such as citric acid and lactic acid, by fermentation are also known in the art. See, e.g., Papagianni (2007) Biotechnol. Adv. 25:244-63; John et al. (2009) Biotechnol. Adv. 27:145-52.

The saccharification and fermentation processes may be carried out as an SSF process. An SSF process can be conducted, in come embodiments, with fungal cells that express and secrete a variant glucoamylase continuously throughout SSF. The fungal cells expressing variant glucoamylase also can be the fermenting microorganism, e.g., an ethanologenic microorganism. Ethanol production thus can be carried out using a fungal cell that expresses sufficient variant glucoamylase so that less or no enzyme has to be added exogenously. The fungal host cell can be selected from an appropriately engineered fungal strains. Fungal host cells that express and secrete other enzymes, in addition to variant glucoamylase, also can be used. Such cells may express amylase and/or a pullulanase, phytase, alpha-glucosidase, isoamylase, beta-amylase cellulase, xylanase, other hemicellulases, protease, beta-glucosidase, pectinase, esterase, redox enzymes, transferase, or other enzymes. Fermentation may be followed by subsequent recovery of ethanol.

### D. Fermentation Products

The term “fermentation product” means a product produced by a process including a fermentation process using a fermenting organism. Fermentation products contemplated according to the invention include alcohols (e.g., arabinitol, butanol, ethanol, glycerol, methanol, ethylene glycol, propylene glycol, butanediol, glycerin, sorbitol, and xylitol); organic acids (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, oxaloacetic acid, propionic acid, succinic acid, and xylonic acid); ketones (e.g., acetone); amino acids (e.g., aspartic acid, glutamic acid, glycine, lysine, serine, and threonine); an alkane (e.g., pentane, hexane, heptane, octane, nonane, decane, undecane, and dodecane); a cycloalkane (e.g., cyclopentane, cyclohexane, cycloheptane, and cyclooctane); an alkene (e.g. pentene, hexene, heptene, and octene); gases (e.g., methane, hydrogen (H₂), carbon dioxide (CO₂), and carbon monoxide (CO)); antibiotics (e.g., penicillin and tetracycline); enzymes; vitamins (e.g., riboflavin, B₁₂, beta-carotene); and hormones.

In a preferred aspect the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred fermentation processes used include alcohol fermentation processes, which are well known in the art. Preferred fermentation processes are anaerobic fermentation processes, which are well known in the art.

### E. Brewing

Processes for making beer are well known in the art. See, e.g., Wolfgang Kunze (2004) “Technology Brewing and Malting” Research and Teaching Institute of Brewing, Berlin (VLB), 3rd edition. Briefly, the process involves: (a) preparing a mash, (b) filtering the mash to prepare a wort, and (c) fermenting the wort to obtain a fermented beverage, such as beer.

The brewing composition comprising a variant glucoamylase, in combination with an amylase and optionally a pullulanase and/or isoamylase, may be added to the mash of step (a) above, i.e., during the preparation of the mash. Alternatively, or in addition, the brewing composition may be added to the mash of step (b) above, i.e., during the filtration of the mash. Alternatively, or in addition, the brewing composition may be added to the wort of step (c) above, i.e., during the fermenting of the wort.

### F. Animal Nutrition

The variant glucoamylases and the compositions described herein can be used as a feed additive for animals to increase starch digestibility. Describe herein is a method for increasing starch digestibility in an animal.

The term “animal” refers to any organism belonging to the kingdom Animalia and includes, without limitation, mammals (excluding humans), non-human animals, domestic animals, livestock, farm animals, zoo animals, breeding stock and the like. For example, there can be mentioned all non-ruminant and ruminant animals. In an embodiment, the animal is a non-ruminant, i.e., a mono-gastric animal. Examples of mono-gastric animals include, but are not limited to, pigs and swine, such as piglets, growing pigs, sows; poultry such as turkeys, ducks, chicken, broiler chicks, layers; fish such as salmon, trout, tilapia, catfish and carps; and crustaceans such as shrimps and prawns. In a further embodiment, the animal is a ruminant animal including, but not limited to, cattle, young calves, goats, sheep, giraffes, bison, moose, elk, yaks, water buffalo, deer, camels, alpacas, llamas, antelope, pronghorn and nilgai.

The terms “animal feed”, “feed”, “feedstuff” and “fodder” are used interchangeably and can comprise one or more feed materials selected from the group comprising a) cereals, such as small grains (e.g., wheat, barley, rye, oats and combinations thereof) and/or large grains such as maize or sorghum; b) byproducts from cereals, such as corn gluten meal, Distillers Dried Grains with Solubles (DDGS) (particularly corn based Distillers Dried Grains with Solubles (cDDGS), wheat bran, wheat middlings, wheat shorts, rice bran, rice hulls, oat hulls, palm kernel, and citrus pulp; c) protein obtained from sources such as soya, sunflower, peanut, lupin, peas, fava beans, cotton, canola, fish meal, dried plasma protein, meat and bone meal, potato protein, whey, copra, sesame; d) oils and fats obtained from vegetable and animal sources; and/or e) minerals and vitamins.

The digestibility of starch in feeds is highly variable and dependent on a number of factors including the physical structure of both the starch and feed matrix. It has been found that starch digestibility in an animal's diet can be improved by the use of at least one glucoamylase as a feed additive.

When used as, or in the preparation of, a feed, such as functional feed, the enzyme or feed additive composition of the present invention may be used in conjunction with one or more of: a nutritionally acceptable carrier, a nutritionally acceptable diluent, a nutritionally acceptable excipient, a nutritionally acceptable adjuvant, a nutritionally active ingredient. For example, there could be mentioned at least one component selected from the group consisting of a protein, a peptide, sucrose, lactose, sorbitol, glycerol, propylene glycol, sodium chloride, sodium sulfate, sodium acetate, sodium citrate, sodium formate, sodium sorbate, potassium chloride, potassium sulfate, potassium acetate, potassium citrate, potassium formate, potassium acetate, potassium sorbate, magnesium chloride, magnesium sulfate, magnesium acetate, magnesium citrate, magnesium formate, magnesium sorbate, sodium metabisulfite, methyl paraben and propyl paraben.

It is also possible that at least one variant glucoamylase (or an enzyme composition comprising at least one variant glucoamylase as described herein) described herein can be homogenized to produce a powder. The powder may be mixed with other components known in the art. Optionally, the feedstuff may also contain additional minerals such as, for example, calcium and/or additional vitamins. In some embodiments, the feedstuff is a corn soybean meal mix.

In an alternative preferred embodiment, an enzyme composition comprising at least one glucoamylase can be formulated to granules as described in WO2007/044968 (referred to as TPT granules) or WO1997/016076 or WO1992/012645 incorporated herein by reference. “TPT” means Thermo Protection Technology. When the feed additive composition is formulated into granules, the granules comprise a hydrated barrier salt coated over the protein core. The advantage of such salt coating is improved thermotolerance, improved storage stability and protection against other feed additives otherwise having adverse effect on the enzyme. Preferably, the salt used for the salt coating has a water activity greater than 0.25 or constant humidity greater than 60% at 20° C. In some embodiments, the salt coating comprises Na₂SO₄.

Alternatively, the composition is in a liquid formulation suitable for consumption preferably such liquid consumption contains one or more of the following: a buffer, salt, sorbitol and/or glycerol.

Any of the glucoamylases described herein for use as a feed additive may be used alone or in combination with at least one direct fed microbial. Categories of DFMs include Bacillus, Lactic Acid Bacteria and Yeasts. Further, any of the glucoamylases described herein for use as a feed additive may be used alone or in combination with at least one essential oil, for example cinnamaldehyde and/or thymol. Still further, any of the glucoamylases described herein for use as a feed additive may be used alone or in combination with at least one additional enzyme. Examples of such enzymes include, without limitation, phytases, xylanases, proteases, amylases, glucanases, or other glucoamylases.

Also disclosed is a method for improving the nutritional value of an animal feed, wherein an effective amount of any of the variant glucoamylases described herein can be added to animal feed.

The phrase, an “effective amount” as used herein, refers to the amount of an active agent (such as any of the variant glucoamylase polypeptides disclosed herein) required to confer improved performance on an animal on one or more metrics, either alone or in combination with one or more other active agents (such as, without limitation, one or more additional enzyme(s), one or more DFM(s), one or more essential oils, etc.).

The term “animal performance” as used herein may be determined by any metric such as, without limitation, the feed efficiency and/or weight gain of the animal and/or by the feed conversion ratio and/or by the digestibility of a nutrient in a feed and/or digestible energy or metabolizable energy in a feed and/or by animals' ability to avoid the negative effects of diseases or by the immune response of the subject.

Animal performance characteristics may include but are not limited to: body weight; weight gain; mass; body fat percentage; height; body fat distribution; growth; growth rate; egg size; egg weight; egg mass; egg laying rate; mineral absorption; mineral excretion, mineral retention; bone density; bone strength; feed conversion rate (FCR); average daily feed intake (ADFI); Average daily gain (ADG) retention and/or a secretion of any one or more of copper, sodium, phosphorous, nitrogen and calcium; amino acid retention or absorption; mineralization, bone mineralization carcass yield and carcass quality.

By “improved animal performance on one or more metric” it is meant that there is increased feed efficiency, and/or increased weight gain and/or reduced feed conversion ratio and/or improved digestibility of nutrients or energy in a feed and/or by improved nitrogen retention and/or by improved ability to avoid the negative effects of necrotic enteritis and/or by an improved immune response in the subject resulting from the use of feed comprising the feed additive composition described herein as compared to a feed which does not comprise said feed additive composition.

All references cited herein are herein incorporated by reference in their entirety for all purposes. In order to further illustrate the compositions and methods, and advantages thereof, the following specific examples are given with the understanding that they are illustrative rather than limiting.

### EXAMPLES

### Example 1

Site Evaluation Library (SEL) Construction, Transformation and Expression of Mucorales-Clade Variant Glucoamylases in Trichoderma reesei

The polynucleotides (codon modified sequences used as expression cassettes) encoding Mucorales-clade variant glucoamylases were synthesized by Generay (Generay Biotech Co., Ltd, Shanghai, China) and inserted into the pI1c expression vector, a derivative vector from pTTT (see, Published US Patent Application 20110020899), that lacks telomeric regions and the acetoamidase marker. Generay Seamless cloning reagents were used as described by vendor. In these studies, the gene sequence of the parent glucoamylase molecule is SEQ ID NO:1, and the translation product is SEQ ID NO:2. The predicted mature sequence of the parent glucoamylase molecule for this site evaluation study is SEQ ID NO:3.

All fungal manipulations, including high throughput transformations, inoculations, fermentations and harvesting were performed in 96 well microtiter plates (MTPs). Plasmids were transformed into suitable T. reesei host strain using the polyethylene glycol (PEG)-protoplast method, omitting plating on solid agar. In brief, transformation mixtures containing approximately 0.5-2 μg of DNA, 2×10⁶ protoplasts and 4 pmol ribonucleoprotein (RNP) in a total volume of 60 μL were treated with 150 μL of 25% PEG solution. Protoplasts were regenerated in a liquid, selective growth medium containing 0.75 M sorbitol to maintain osmotic pressure. Plates with regenerated fungal cultures were grown for 4 days in a shaker incubator with a 50 mm throw at 200 rpm and 28° C. with 80% humidity until fungal mycelia was formed. This material was used to inoculate cultures for expression of glucoamylase variants.

For the expression of glucoamylase proteins, the transformed T. reesei strains were cultured as follows: 40 μl of regenerated transformants were used to inoculate 360 μl of culture medium containing glucose, (NH₄)₂SO₄, PIPPS buffer (pH 5.5), salts and trace elements. The cultures were grown in 96 well MTPs incubated in a shaker incubator with a 50 mm throw at 260 rpm and 28° C. with 80% humidity. After 2 days of pre-culturing, the cultures were transferred to a culture medium similar to one described above, where the glucose was replaced with a glucose/sophorose mixture. The 96 well MTPs were subsequently incubated in a shaker incubator with a 50 mm throw at 260 rpm and 28° C. with 80% humidity. After 5 days of fermentation the cultures were filtered by centrifugation using hydrophilic PVDF membranes to obtain clarified supernatants used for analysis of the recombinant glucoamylase enzymes.

Supernatants from the transformation screen were subjected to protein quantification. A 5 μl aliquot of clarified culture supernatant from MTP cultures was injected on a Zorbax 300 C3 Rapid Resolution HD 2.1×50 mm 1.8-Micron (Agilent) column at 80° C. Glucoamylases were eluted with a 3 min gradient of buffer A (0.1% TFA in water) (and buffer B (0.07% TFA, 70% acetonitril, 30% isopropanol). Glucoamylase protein concentrations were determined using a calibration curve (0-1024 ppm) of a fermentation sample with a known glucoamylase concentration. For the activity assays reported on Table 1 (Example 2 below), samples were normalized in 20 mM sodium acetate buffer pH 4.5 containing 0.005% (v/v) Tween-80 to a concentration of 200 ppm for the saccharification assay, 40 ppm for the reversion assay and 15 ppm for all colorimetric ABTS assays. Calibration curves of the parent molecule with concentration ranging from 0-80 ppm were included in the assay plates for use in performance index (PI) calculations.

A representative sample of culture supernatant of the parent glucoamylase was analysed by mass spectroscopy (MS). Results of the MS analysis shows the presence of multiple polypeptides. The predicted C-terminus of SEQ ID NO: 3 was confirmed while several truncations of the predicted N-terminus were detected. Truncations after N-terminal residues: 1 (SEQ ID NO: 5), residue 2 (SEQ ID NO: 35), residue 3 (SEQ ID NO: 6), residue 5 (SEQ ID NO: 36), and residue 6 (SEQ ID NO: 37) were observed.

### Example 2

### Evaluation of the Enzymatic Activity of Mucorales-Clade Glucoamylase Variants in Saccharification-Relevant Assays

The saccharification performance of variants and the parent molecule (SEQ ID NO:3) was evaluated at pH 4.5 and 62° C. over 48 hr using Maltosweet G 120 as substrate (DE=11−14) purchased from Tate & Lyle. Starting DS content of saccharification blend was 34%. The performance of the glucoamylases was tested at the dosage of 28 μg/gds. For this evaluation, the pullulanase OPTIMAX™ L 1000 (an IFF product) was dosed at 0.438 ASPU/g DS and the alpha-amylase Aspergillus terreus amylase (described in International Patent Application Publication No. WO2014099415, incorporated by reference herein) was dosed at 0.227 SSU/g DS for each incubation. Reactions consisting of 75 μl of maltodextrin and 5 μl of saccharification blends (as described above, glucoamylase, alpha-amylase and pullulanase) were incubated in MTP at pH 4.5, 62° C. for 48 hr with shaking at 900 rpm. All the reactions were quenched by addition of 5 mM H₂SO₄ in a ratio of 1:40 saccharification syrup to acid solution. Quenched aliquots were analyzed by HPLC for product formation using an Agilent 1260 series system with a double Phenomenex Rezex-RFQ Fast Acid H+(8%) 100×7.8 mm column (cat #00D-0223-K0) running at 80° C. for 5.3 minutes. For this purpose, 5 μL sample was injected on the column and carbohydrates were separated with an isocratic gradient of 5 mM H₂SO₄ as the mobile phase at a flow rate of 1.0 mL/min. The oligosaccharide products were detected using a refractive index detector. Standards were run to determine elution times of each degree of polymerization (DP(n)) of sugars of interest (where n represents the number of saccharides, e.g., DP3+, DP3, DP2 and DP1). The total integrated peak area of the identified saccharification products was used to calculate the relative content of each (oligo)saccharide. The performance of GA variants in saccharification was compared against that of the parent molecule (SEQ ID NO:3). To determine the impact of an amino acid substitution on this GA property, an increase in % glucose yield over the parent molecule was determined and the results are shown on Table 1. Values larger than 0.5% are considered a positive effect of the mutation on saccharification.

### Specific Activity Determination Using Colorimetric Read Out

Glucoamylase variant specific activity was determined based on the release rate of glucose from selected substrates using the coupled glucose oxidase/peroxidase (GOX/HRP) and 2,2′-Azino-bis 3-ethylbenzothiazoline-6-sulfonic acid (ABTS) method (described in Anal. Biochem. 105 (1980), 389-397).

A 5 μL aliquot of glucoamylase supernatant was combined in an MTP with 25 μl freshly prepared reagent A solution containing 25 mM sodium acetate buffer (pH 4.5), with final concentrations 25 U/mL HRP (Sigma-Aldrich, Prod.nr: P8375), 62 U/mL GOX (Sigma-Aldrich, Prod.nr: G7141) and 0.005% Tween-80. To initiate the reaction 20 μl of substrate-containing reagent B was added consisting of 5.4 mg/mL ABTS (Sigma-Aldrich, Prod.nr: A1888), 25 mM sodium acetate buffer (pH 4.5) and 0.005% Tween-80. Final substrate concentrations of the various assays were: 4.6 mM maltose (Sigma-Aldrich, Prod.nr: 47288), 30 mM panose (Megazymes, O-PAN), 1% pre-boiled (w/v) pullulan (Sigma-Aldrich, Prod.nr: P4516), 0.5% pre-boiled (w/v) soluble starch (Sigma-Aldrich, Prod.nr: S9765) and 1% (w/v) maltodextrin (Sigma-Aldrich, Prod.nr: 31410). Reagents were mixed vigorously, and the absorbance kinetics were read immediately for 6 minutes at 23 seconds intervals, with continuous stirring, at room temperature using a plate reader from Molecular Devices. The increase of the absorbance rate at 405 nm is proportional to the hydrolytic activity of the enzyme. Reactions were run in triplicate and a linear regression was used to determine the Vo of the samples. The data generated this way was used to calculate the performance of each sample on each assay. The activity rate of each variant divided by the predicted activity rate of the parent GA at an equivalent protein concentration was defined as Performance Index (PI). A PI value equal to or larger than 1.1 over that of the parent was used as cut-off for the selection of mutations with beneficial effect on the specific activity of the molecule. Results are shown in Table 1.

### Thermostability Evaluation of Glucoamylase Variants

Glucoamylase variant samples set to 15 ppm were incubated in 384 well PCR MTPs at 57° C. for 30 min in a Mastercycler Pro 384 (Eppendorf). After incubation, the MTPs were cooled to room temperature and were used to determine the specific activity on maltodextrin substrate using the ABTS method described above. The percent (%) residual activity (activity remaining after heat treatment) of the glucoamylase variants was determined by dividing the activity of the heat-incubated samples with that of non-incubated samples. An improved thermostability of greater than 5% above that of the parent enzyme was used as cut-off in the selection of mutations with beneficial effect on the thermostability of the molecule. The thermostability results are shown in Table 1 as % Residual Activity.

### Reversion Reaction Evaluation of Glucoamylase Variants

Glucose condensation activity (reversion activity) for each GA variant was tested by combining 67 μl of 50% (w/v) glucose in 10 mM sodium acetate buffer pH 4.5 with 13 μl of an enzyme sample in 384 well MTPs and incubated with shaking at 50° C. for 66 hours. The reactions were quenched by mixing 2.5 μl of reaction cocktail with 97.5 μl of 5 mM H₂SO₄. Reaction products (glucose and condensation products such as DP2, DP3 and DP3+) were determined and quantified by HPLC analysis as described above.

The performance of GA variants was compared against that of the parent molecule (SEQ ID NO:3). To determine the impact of an amino acid substitution on this GA property, a reduction in reversion yield (DP2) over the parent molecule expressed as % condensation product was determined and the results are shown on Table 1. Values equal to or larger than 0.4 for reduced reversion are considered a positive effect on reversion under the conditions of this assay.

Note that for residual activity comparisons, the values used are absolute (as measured), while for reversion reactions and for saccharification yields the values are calculated as a difference in percent yields (e.g. Variant A51Y has a 93.2% DP1 yield vs 91.8% DP1 for the parent GA, thus giving a saccharification yield improvement yield of 1.4%).

The Mucorales-clade GA variants described in Table 1 all display at least one improved property when compared to the parental sequence SEQ ID NO:3 indicating a benefit for each of the amino acid substitutions. SEQ ID NO:3 is equivalent to SEQ ID NO:4 (SvaGA1) with Pro at position 102. The instances when cells appear blank on Table 1 indicate that a benefit (improvement over parent) was not observed for that particular instance. For instance, the PIs where <1.0, the thermostability % residual activity was less than 5% improved (compared to parent control on assay plate) and the reduced reversion and improved saccharification results were respectively less than 0.4% or 0.5% above the parent. The asterisk next to % residual activity for thermostability of the parent molecule denotes that this is an average of multiple tests across the evaluations performed for the variant molecules.

TABLE 1
Performance of Mucorales-clade glucoamylase variants compared to SEQ ID NO: 3 parent enzyme

| | | | | | | | Reduced | Improved | | |
|---|---|---|---|---|---|---|---|---|---|---|
| | | | | | | | Reversion | saccharification | | Thermostability, |
| | | | | | | | over the | yield | PI on | % |
| | | | PI on | PI on | PI on | PI on | parent GA, | over parent, | Soluble | Residual |
| Parent-AA | Position | Substitution | Maltodextrin | Maltose | Panose | Pullulan | % DP2 | % glucose | starch | Activity |
| SEQ ID | — | — | 1.0 | 1.0 | 1.0 | 1.0 | 0 | 0 | 1.0 | 30* |
| NO: 3 | | | | | | | | | | |
| S | 020 | A | 1.2 | 1.3 | 1.3 | 1.2 | | | | |
| S | 020 | E | 1.2 | | | | | | | |
| S | 020 | F | | | | 1.3 | | | | |
| S | 020 | G | | | | 1.1 | | | | |
| S | 020 | P | 1.3 | 1.5 | 1.4 | | | | | |
| K | 021 | M | 1.1 | | 1.1 | | | | | |
| K | 021 | S | 1.6 | 1.7 | | | | 1.4 | | |
| K | 021 | T | | | 1.7 | | | | 1.7 | |
| K | 021 | W | | | 1.5 | | | 1.1 | | |
| E | 023 | L | | | 1.5 | | | 2.7 | 1.8 | |
| E | 023 | M | | | 2.1 | | | | 1.9 | 36 |
| E | 037 | C | | | 2.0 | | | | 2.2 | |
| E | 037 | N | | | 1.9 | | | 1.2 | 2.0 | 51 |
| S | 049 | W | | | | | 1.3 | | | |
| A | 051 | K | 1.1 | | | 1.3 | | 1.0 | | |
| A | 051 | L | 1.2 | 1.3 | 1.2 | | | | | |
| A | 051 | V | 1.3 | 1.2 | 1.2 | | | | | |
| A | 051 | Y | 1.1 | | | | 1.0 | 1.4 | | 33 |
| G | 052 | F | | | | | | | 1.2 | |
| G | 052 | N | | | 1.6 | | | 2.5 | 1.9 | 49 |
| G | 052 | P | | | | | 1.4 | | | |
| V | 066 | A | | | | | | 2.4 | 1.1 | 65 |
| V | 066 | C | | | | | | 1.7 | | 53 |
| V | 066 | F | | | | | | 1.7 | 1.6 | 57 |
| V | 066 | M | | | | | | 1.3 | | 54 |
| V | 066 | P | | | | | 1.3 | | | |
| V | 066 | T | | | | | | | 1.2 | |
| V | 066 | W | | | | | | 1.7 | | 53 |
| S | 067 | A | 1.1 | | | | | 0.7 | | |
| S | 067 | C | | | 1.2 | | | 0.7 | | 35 |
| S | 067 | M | | | | | | 1.6 | | |
| V | 069 | A | | | 1.2 | | | | | |
| V | 069 | C | 1.1 | | 1.4 | 1.5 | 0.4 | | 1.3 | |
| V | 069 | K | | | | | 0.9 | | | |
| K | 073 | N | 1.2 | | 1.2 | 1.3 | | | 1.3 | |
| K | 073 | P | 1.4 | | | 1.6 | | | 1.1 | |
| T | 077 | M | 1.4 | | | 1.5 | 0.4 | | 1.5 | |
| T | 077 | P | 1.4 | | | 1.2 | 0.4 | | 1.7 | 27 |
| T | 077 | S | | | 1.2 | | | | 1.2 | |
| A | 079 | C | 1.3 | 1.2 | 1.1 | 1.4 | | | 1.1 | 29 |
| A | 079 | R | | | | | | 0.8 | 1.2 | |
| A | 079 | T | 1.3 | 1.1 | | 1.3 | | | | |
| G | 080 | H | 1.1 | | 1.3 | | 0.5 | | 1.2 | |
| G | 080 | K | 1.2 | 1.1 | 1.2 | 1.3 | | | 1.2 | |
| G | 080 | N | | | | | | | 1.3 | |
| D | 081 | N | 1.2 | | 1.2 | 1.2 | | | 1.2 | |
| D | 081 | S | | | | | | 1.0 | 1.2 | |
| I | 084 | L | 1.2 | | 1.3 | 1.2 | | | 1.4 | |
| I | 084 | V | 1.1 | | 1.6 | 1.4 | | | 1.3 | |
| V | 092 | C | | | 1.2 | | | 1.0 | | |
| V | 092 | I | | | | | | | 1.2 | |
| V | 092 | M | | 1.1 | | | | 0.9 | | |
| F | 094 | A | | | | 1.2 | 1.5 | | | |
| F | 094 | G | | | | 1.3 | 1.5 | | | |
| F | 094 | Y | | | 1.2 | | | | | |
| P | 102 | G | 1.5 | 1.7 | 1.5 | | | | | |
| S | 119 | A | | | | | | 1.1 | 1.2 | 46 |
| S | 119 | D | | | | 1.4 | 1.3 | | | |
| S | 119 | G | 1.3 | | 1.5 | 1.5 | | | 1.1 | |
| T | 121 | G | 1.3 | 1.6 | 1.4 | | | | | |
| T | 121 | L | | | 1.7 | 1.4 | | | 1.6 | |
| T | 121 | M | 1.5 | 1.2 | 2.0 | 1.3 | | | 2.2 | 32 |
| T | 121 | P | 1.1 | 1.5 | 1.1 | | | | | |
| T | 121 | V | 1.1 | | | | | | | |
| E | 134 | Q | 1.2 | | | | 1.0 | | 1.2 | |
| E | 134 | S | 1.6 | | | 1.4 | | | 1.9 | 44 |
| E | 134 | W | | | | | 1.5 | | | |
| M | 140 | C | 1.6 | 1.3 | 1.6 | 1.5 | | | 1.7 | 33 |
| M | 140 | I | 1.4 | 1.5 | 1.4 | 1.6 | | 1.8 | 1.6 | 35 |
| M | 140 | Q | | | 1.6 | 1.5 | | | 1.5 | |
| L | 141 | F | 1.2 | | 1.3 | 1.6 | | | 1.8 | 36 |
| L | 141 | K | | | | | | | 1.2 | |
| A | 143 | G | 1.3 | 1.1 | 1.4 | 1.5 | | | 2.0 | 32 |
| F | 156 | C | 1.1 | | | 1.3 | | | | 46 |
| F | 156 | P | 1.2 | | | 1.4 | | | | |
| T | 157 | A | | | | | | | 1.2 | |
| T | 157 | I | 1.6 | 1.7 | | | | | | 26 |
| N | 158 | A | 1.3 | 1.2 | | 1.4 | | 1.3 | 1.1 | |
| N | 158 | Y | | | | | 0.4 | 1.0 | | |
| I | 164 | L | | | 1.9 | | 0.8 | 1.0 | 2.5 | |
| I | 164 | T | | | | | 1.8 | | 1.8 | |
| Y | 165 | I | | | | | 0.4 | | | 42 |
| Y | 165 | T | 1.3 | | 1.2 | 1.5 | | | 1.3 | |
| Y | 165 | W | 1.4 | 1.3 | 1.4 | | 0.6 | | 1.6 | 43 |
| K | 166 | A | | | | 1.4 | | | | |
| K | 166 | F | | 1.2 | | 1.3 | | | | |
| K | 166 | G | 1.1 | 1.3 | | | | | | |
| K | 166 | H | | | | 2.0 | | | | |
| K | 166 | R | | 1.2 | 1.2 | | | | | |
| V | 172 | G | 1.6 | | 1.5 | | 0.5 | 1.5 | | |
| V | 172 | L | | | | | | 1.6 | | |
| Y | 192 | F | 1.8 | 2.0 | | | 0.4 | 1.3 | | 26 |
| Y | 192 | R | | | | | 1.5 | | | |
| D | 203 | C | 1.2 | | | 1.5 | | | | |
| D | 203 | M | 1.1 | | | | | | | |
| D | 203 | Q | | | | 1.1 | | | | |
| D | 203 | W | 1.3 | 1.1 | | 1.1 | | | | |
| D | 203 | Y | | | | 1.1 | | | | |
| R | 210 | A | | | | 1.2 | | | | |
| R | 210 | F | | | | 1.4 | | | | |
| R | 210 | G | | | | | | | 1.6 | |
| R | 210 | I | | | | 1.2 | | | | |
| R | 210 | L | 1.3 | 1.7 | 1.5 | 1.8 | | | | |
| R | 210 | M | 1.2 | | | 1.5 | | | | |
| R | 210 | N | | | | 1.3 | | | | |
| R | 210 | W | | | | | | 1.9 | 1.7 | |
| D | 213 | H | | | 1.9 | | 0.7 | 1.2 | 3.0 | |
| D | 213 | R | 1.7 | 2.1 | | | | | | 25 |
| N | 214 | A | 1.3 | 1.4 | 1.3 | 1.4 | | | | |
| N | 214 | C | | | | 1.2 | | | | |
| N | 214 | E | 1.2 | 1.3 | 1.3 | | | | | |
| N | 214 | G | | | | | | | 1.2 | |
| N | 214 | L | | | | 1.2 | | | | |
| N | 214 | T | | | | | 1.4 | 1.2 | | |
| N | 214 | Y | | | | 1.3 | | 1.2 | 1.1 | |
| S | 215 | C | 1.2 | 1.1 | | 1.2 | | 1.3 | | |
| S | 215 | D | 1.2 | 1.4 | 1.4 | 1.1 | | | | |
| S | 215 | F | 1.1 | 1.1 | | | | 1.4 | | |
| S | 215 | H | | | | 1.1 | | | | |
| S | 215 | R | | | | | 0.5 | 1.2 | | |
| S | 215 | V | | | | 1.2 | | | | |
| S | 215 | W | | | | 1.2 | | | | |
| S | 218 | A | | | 1.2 | 1.2 | | 1.2 | 1.1 | |
| A | 218 | F | | | | | | | 1.4 | |
| S | 218 | H | | | | | | 1.6 | | |
| S | 218 | K | | | | 1.4 | 0.7 | 1.3 | | |
| S | 218 | Q | 1.1 | | 2.1 | 1.2 | | | 2.3 | |
| S | 218 | V | 1.1 | | 1.1 | 1.5 | | | | |
| S | 218 | Y | 1.1 | | 1.1 | | | | | |
| S | 221 | M | | 1.1 | | | | 1.7 | | |
| S | 221 | R | 1.3 | 1.2 | 1.9 | 1.3 | | | 2.0 | 37 |
| S | 221 | T | 1.1 | 1.1 | | | | 1.1 | | |
| G | 222 | C | 1.1 | 1.1 | 1.2 | | 0.5 | | 1.3 | |
| G | 222 | M | 1.4 | 1.2 | 2.0 | 1.3 | | | 2.1 | 36 |
| G | 222 | V | | | 1.5 | | | 1.3 | 1.4 | |
| S | 233 | M | 1.6 | 1.3 | 2.0 | 1.7 | | | 2.2 | 37 |
| S | 233 | P | 1.3 | 1.1 | 1.3 | 1.6 | 0.5 | | 1.3 | |
| S | 233 | T | | | 1.4 | | 1.5 | 1.0 | | |
| S | 233 | Y | 1.1 | 1.1 | | | | | | |
| W | 235 | F | | | | | 1.2 | 0.8 | 1.4 | |
| W | 235 | Y | 1.1 | 1.1 | | 1.2 | | 1.1 | | |
| D | 236 | A | 1.2 | 1.2 | | | 0.6 | 1.2 | | |
| D | 236 | Q | 1.4 | 1.2 | 2.0 | 1.2 | | | 2.0 | 30 |
| D | 236 | S | | | | | | 1.4 | | |
| G | 238 | H | | 1.2 | 1.2 | | 0.9 | | | |
| G | 238 | M | | | | | | 0.6 | | |
| G | 238 | N | | | | | 0.8 | | | |
| G | 238 | S | | 1.3 | | | 0.8 | | | |
| G | 238 | T | | | | | | 1.6 | | |
| G | 238 | V | | | | | | 0.8 | | |
| T | 243 | A | | | | | | 0.8 | 1.2 | |
| T | 243 | E | | 1.2 | | | | | | |
| T | 243 | P | | | | | 0.7 | | | |
| T | 243 | S | | | | | 0.6 | | | |
| T | 243 | V | | 1.1 | | | | | | |
| V | 252 | C | | 1.2 | | | | | | |
| V | 252 | F | 1.3 | 1.1 | 1.8 | 1.2 | | | 1.8 | 27 |
| V | 252 | H | | | | 1.1 | | | | |
| E | 253 | F | | | 1.8 | | | | 2.2 | 33 |
| E | 253 | K | | | | 1.2 | 0.4 | 1.5 | 1.2 | |
| G | 274 | A | 1.3 | 1.1 | | | 0.5 | | 1.1 | |
| G | 274 | D | 1.2 | | 2.0 | 1.2 | | | 1.9 | |
| G | 274 | K | 1.3 | | 1.3 | 1.6 | 1.0 | | 1.3 | |
| P | 278 | A | | 1.4 | | 1.2 | | | | |
| E | 281 | D | 1.3 | 1.3 | 1.5 | 1.5 | | 1.0 | 1.7 | 31 |
| E | 281 | P | | | | | | 1.0 | | |
| F | 290 | M | 1.3 | 1.3 | 1.7 | 1.5 | | | 2.0 | |
| F | 290 | V | 1.1 | 1.3 | 1.5 | | | 1.5 | 1.6 | 27 |
| N | 302 | F | | | | | 1.2 | | | |
| N | 302 | H | | | | | 1.3 | 1.5 | | 63 |
| N | 302 | K | 1.2 | 1.9 | 1.8 | | | 2.1 | 1.2 | 22 |
| N | 302 | M | | | | | 0.8 | | | |
| N | 302 | P | 1.5 | 1.3 | | 1.8 | 1.0 | | | |
| N | 302 | Q | 1.1 | | 1.5 | | 0.6 | 1.6 | 1.9 | 37 |
| N | 302 | S | 1.2 | 1.2 | 1.9 | 1.4 | | | 2.0 | 29 |
| N | 302 | T | | | | | 0.5 | | | |
| N | 302 | V | 1.1 | 1.3 | 1.3 | 1.3 | | | | |
| N | 302 | W | 1.1 | 1.2 | 1.7 | | | 1.0 | 2.0 | 31 |
| N | 310 | T | 1.1 | 1.1 | | 1.2 | | 1.1 | | |
| N | 310 | V | | | | | | 1.4 | | |
| N | 310 | Y | 1.1 | 1.1 | | | | | | |
| N | 321 | D | 1.3 | 1.3 | 1.8 | 1.2 | | | 1.7 | 31 |
| F | 338 | I | | | | | 0.4 | | | |
| L | 341 | M | 1.3 | 1.3 | 2.0 | 1.3 | | | 2.1 | |
| L | 341 | T | | | 1.6 | | | | 1.6 | |
| K | 350 | C | | | 1.6 | | | | 1.6 | 26 |
| K | 350 | E | | | | | | | 1.2 | 39 |
| K | 350 | I | 1.2 | 1.1 | | 1.3 | | | | |
| K | 350 | T | 1.3 | 1.3 | 1.4 | 1.3 | | | 1.8 | 31 |
| N | 351 | E | | | 1.6 | | | | 1.7 | |
| N | 351 | V | 1.1 | | | | | 0.8 | | |
| T | 352 | D | 1.4 | 1.2 | 1.9 | 1.2 | | | 2.0 | 30 |
| T | 352 | N | | | 1.2 | | | | 1.2 | |
| T | 352 | S | | | | 1.2 | | 1.4 | | |
| V | 354 | L | 1.3 | 1.1 | | 1.3 | | 1.2 | | |
| V | 354 | M | 1.1 | 1.1 | | | | | | |
| S | 370 | M | 1.4 | 1.2 | 1.9 | 1.5 | | | 1.9 | 38 |
| S | 370 | R | | | | | | 0.8 | | |
| S | 390 | D | 1.1 | | 2.1 | 1.3 | | | 2.2 | 31 |
| S | 390 | E | 1.3 | 1.7 | 1.6 | 1.2 | | | | |
| S | 390 | L | 1.2 | | 2.0 | 1.2 | | | 2.1 | 33 |
| Q | 403 | G | 1.1 | 1.1 | 2.0 | | 0.9 | | 2.3 | |
| Q | 403 | K | 1.2 | | 1.4 | | | 0.8 | 1.3 | |
| Q | 403 | R | 1.1 | 1.1 | 1.9 | | | | 2.1 | 37 |
| Y | 404 | I | 1.2 | | 2.1 | 1.3 | | | 2.4 | 36 |
| Y | 404 | K | 1.2 | 1.1 | 2.0 | 1.2 | | | 2.0 | 44 |
| Y | 404 | M | 1.1 | | 2.1 | | | | 2.3 | 33 |
| H | 405 | Q | 1.8 | 1.7 | 1.4 | | 1.6 | | | |
| H | 405 | S | 1.5 | 1.2 | 2.0 | | 0.8 | | 1.4 | |
| H | 405 | Y | 1.5 | 1.1 | | | | | | |
| F | 416 | C | | | | | 1.4 | | 1.2 | |
| F | 416 | Y | | | | 1.2 | 1.2 | 1.5 | 1.2 | 58 |
| R | 418 | E | 1.3 | 1.1 | 2.1 | 1.4 | 0.5 | | 2.2 | |
| R | 418 | W | 1.5 | 1.5 | 1.4 | 1.3 | 1.3 | | 1.4 | |
| Y | 422 | A | 1.2 | | | | 0.6 | | 1.3 | 38 |
| Y | 422 | F | | | | | | 1.6 | | |
| Y | 422 | V | | | | | | | | 23 |
| T | 430 | A | | | 4.8 | | 1.5 | 2.3 | | |
| T | 430 | Q | | | | | 0.9 | | | |
| A | 434 | S | 1.3 | | | 1.3 | | | | 28 |
| A | 440 | G | 1.2 | 1.2 | 2.1 | 1.2 | | | 2.0 | |
| A | 440 | H | 1.1 | 1.2 | | | 1.4 | | | |
| A | 440 | L | | | | | 1.4 | | | |
| Q | 441 | L | | | | | 0.4 | 1.3 | 2.5 | |
| Q | 441 | S | | 1.1 | | | | | | |
| Q | 441 | W | | | | | 1.6 | | | |
| A | 444 | L | | 1.1 | | | 1.2 | | | |
| A | 444 | P | 1.3 | | 1.3 | | 0.9 | | 1.1 | |
| A | 444 | S | | | | | | | 1.1 | |
| G | 445 | M | 1.2 | | 2.1 | 1.3 | | | 2.4 | |
| G | 445 | Y | | | 2.8 | | 1.0 | 1.5 | | |
| F | 449 | L | | | | | 1.3 | | | |
| F | 449 | Y | 1.2 | 1.1 | 2.0 | 1.2 | | | 2.0 | 29 |

### Example 3

### Comparison of Glucoamylase Sequences

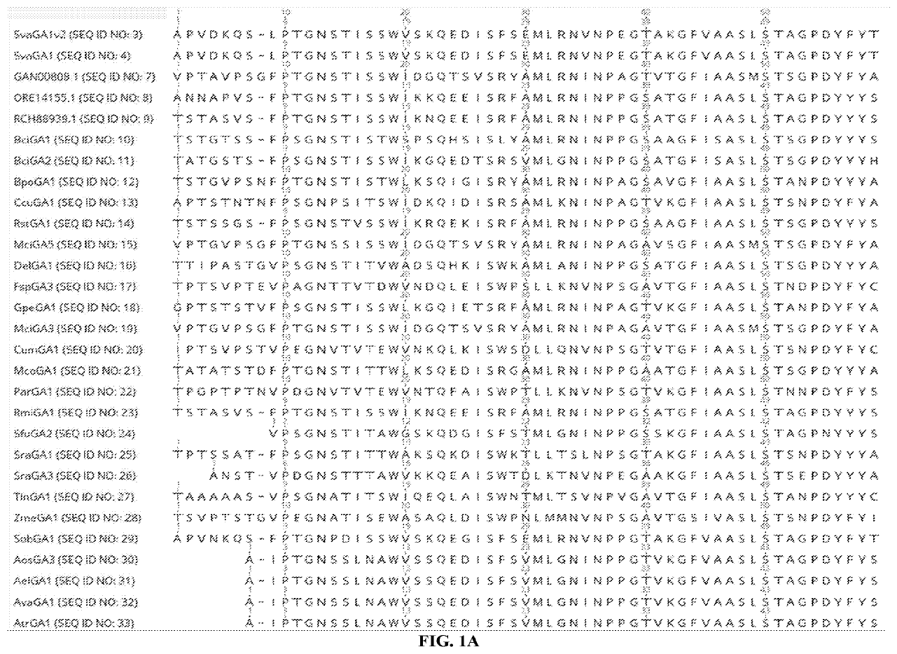

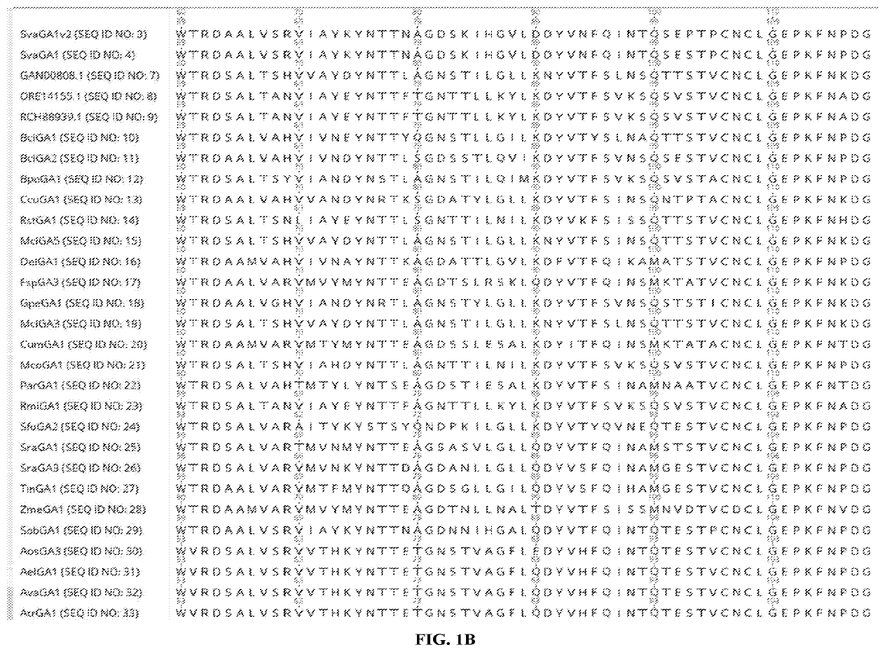

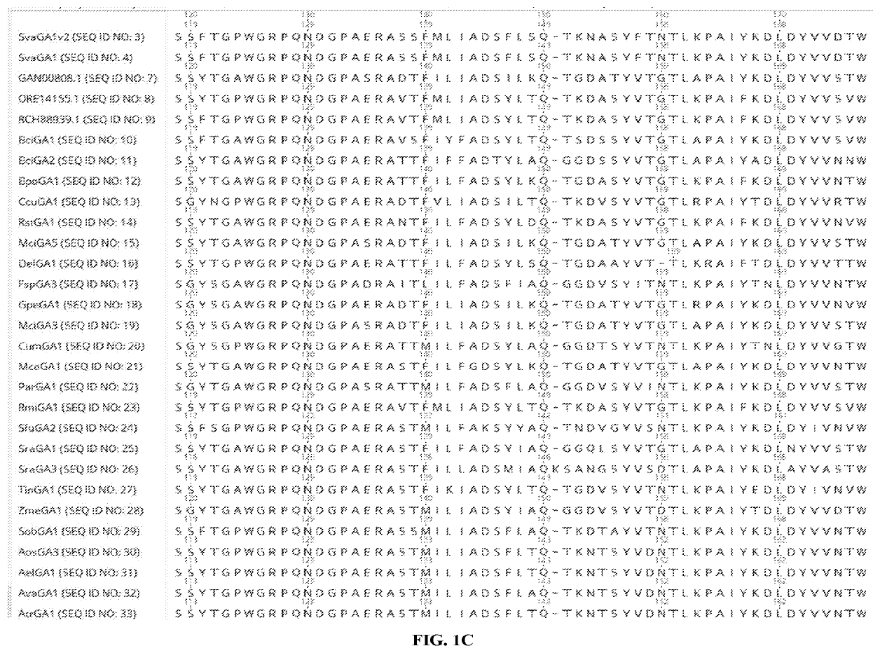

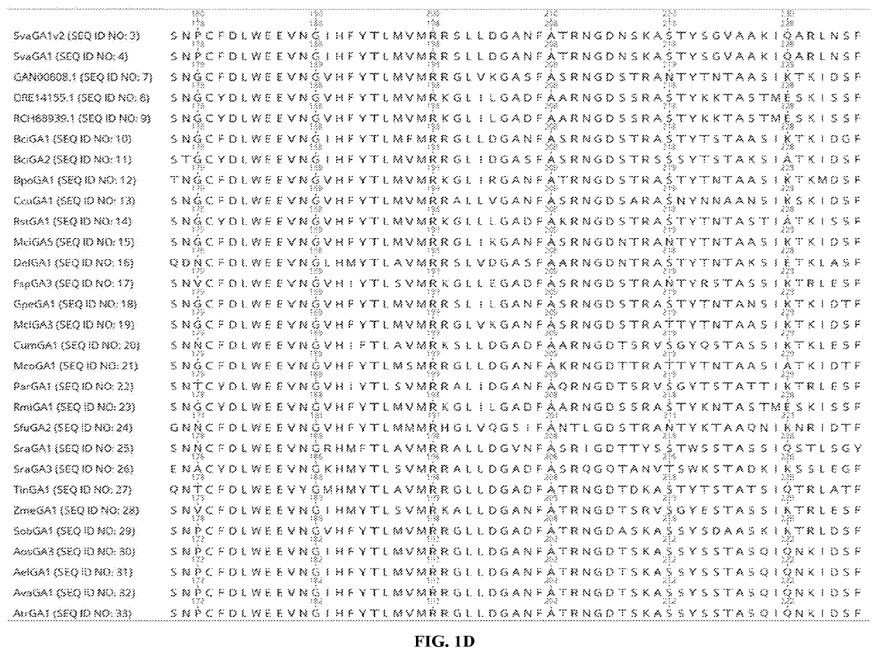

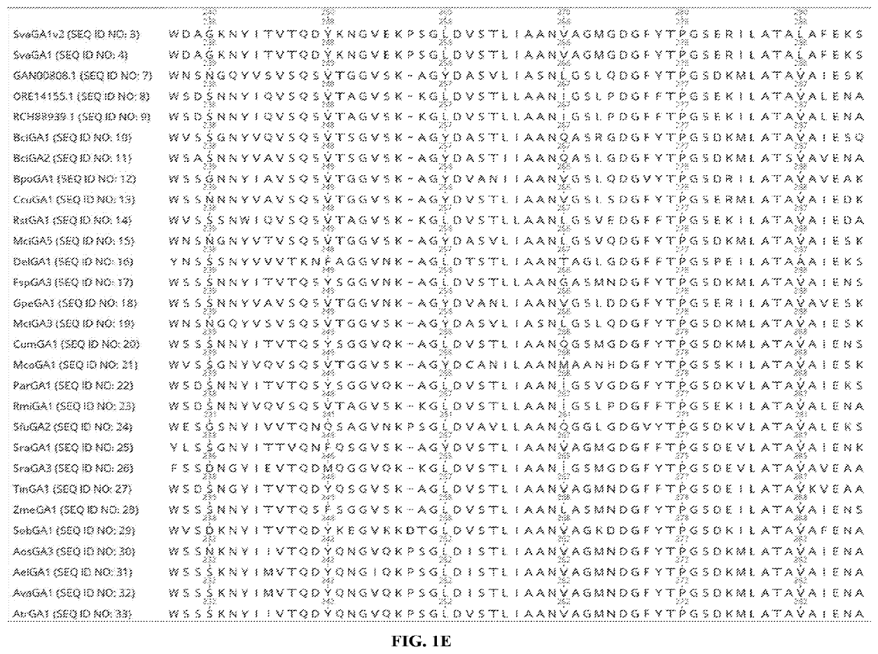

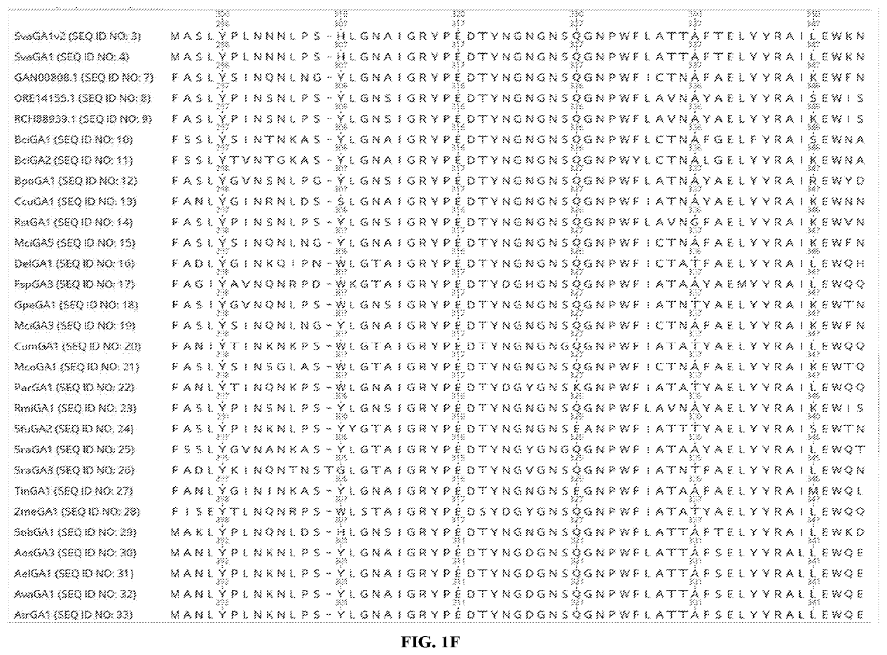

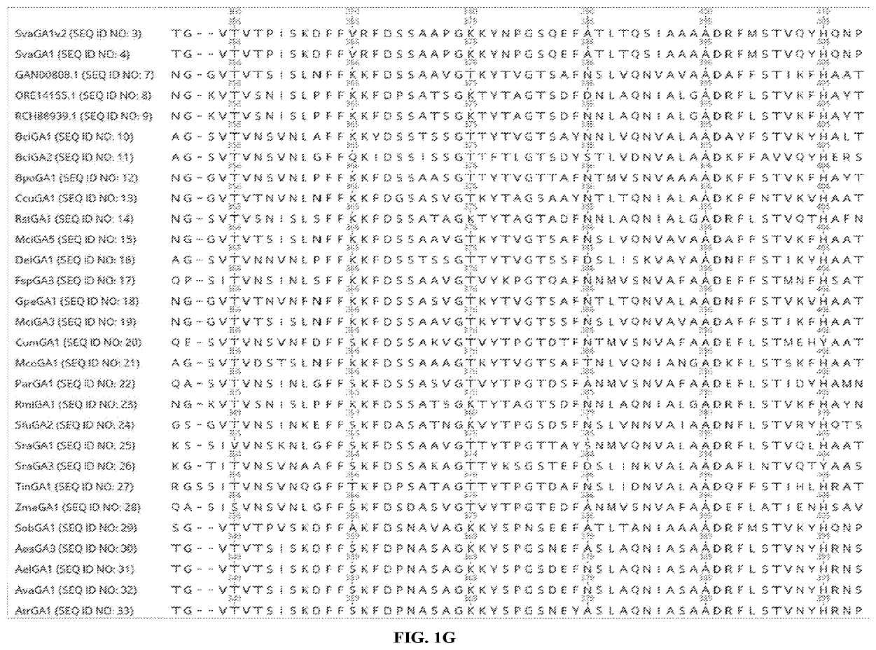

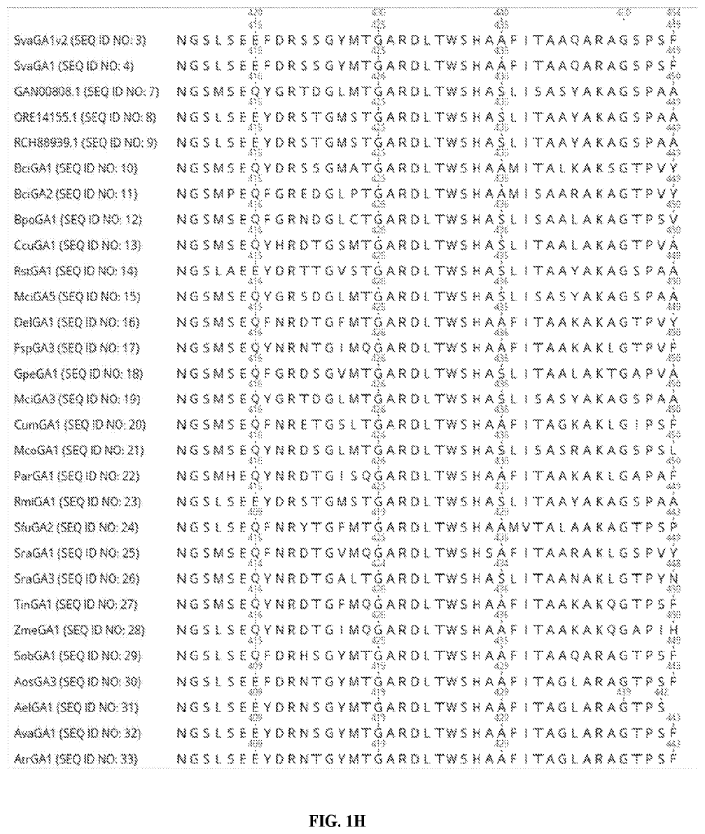

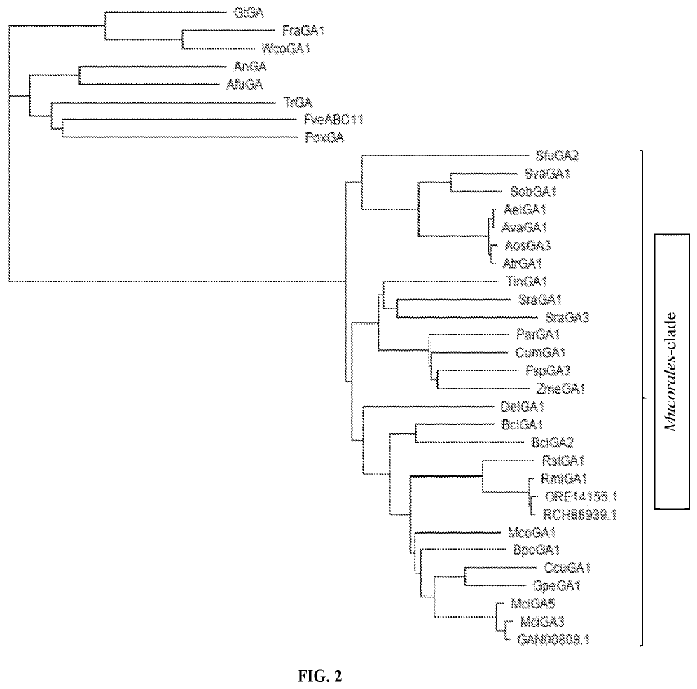

A multiple amino acid sequence alignment was constructed for the predicted mature regions of SEQ ID NO:3 (SvaGA1v2) and SEQ ID NO:4 (SvaGA1) and related glucoamylases: GAN00808.1 (SEQ ID NO:7), ORE14155.1 (SEQ ID NO:8), RCH88939.1 (SEQ ID NO: 9), AelGA1 (SEQ ID NO:31), AosGA3 (SEQ ID NO:30), AtrGA1 (SEQ ID NO: 33), AvaGA1 (SEQ ID NO: 32), BciGA1 (SEQ ID NO:10), BciGA2 (SEQ ID NO: 11), BpoGA1 (SEQ ID NO: 12), CcuGA1 (SEQ ID NO:13), CumGA1 (SEQ ID NO:20), DelGA1 (SEQ ID NO: 16), FspGA3 (SEQ ID NO:17), GpeGA1 (SEQ ID NO:18), MciGA3 (SEQ ID NO: 19), MciGA5 (SEQ ID NO: 15), McoGA1 (SEQ ID NO: 21), ParGA1 (SEQ ID NO:22), RmiGA1 (SEQ ID NO: 23), RstGA1 (SEQ ID NO: 14), SfuGA2 (SEQ ID NO: 24), SobGA1 (SEQ ID NO: 29), SraGA1 (SEQ ID NO: 25), SraGA3 (SEQ ID NO: 26), TinGA1 (SEQ ID NO:27), and ZmeGA1 (SEQ ID NO:28) in order to determine the corresponding amino acid from position 1 to position 449 of SEQ ID NO:3 and 4 with respect to the sequences of these other GAs. These sequences were aligned using MUSCLE alignment tool within Geneious 10.2 software with the default parameters. The multiple sequence alignment is shown on FIG. 1 panels A-H. The percent sequence identity among these GAs calculated from the MUSCLE alignment is shown on Table 2. A phylogenetic tree for these Mucorales-clade GAs and other GAs was generated using Geneious 10.2 software and is shown on FIG. 2.

TABLE 2
Percent identity between various glucoamylases, aligned
within the overlapping region from position
1 to 449 according to SEQ ID NO: 4.

| Glucoamylase | SvaGA1 | SvaGA1v2 (SEQ ID |
|---|---|---|
| identifier | (SEQ ID NO: 4) | NO: 3) |
| SvaGA1 | | 99.8 |
| SvaGA1v2 | 99.8 | |
| GAN00808.1 | 63.2 | 63 |
| ORE14155.1 | 66.4 | 66.2 |
| RCH88939.1 | 66.4 | 66.2 |
| AelGA1 | 78.3 | 78.1 |
| AosGA3 | 78.8 | 78.6 |
| AtrGA1 | 78.8 | 78.6 |
| AvaGA1 | 78.6 | 78.3 |
| BciGA1 | 62.4 | 62.2 |
| BciGA2 | 60.2 | 60 |
| BpoGA1 | 63.9 | 63.6 |
| CcuGA1 | 64.5 | 64.7 |
| CumGA1 | 62.7 | 62.7 |
| DelGA1 | 62.5 | 62.3 |
| FspGA3 | 60.8 | 60.8 |
| GpeGA1 | 64.7 | 64.5 |
| MciGA3 | 62.7 | 62.5 |
| MciGA5 | 64.5 | 64.3 |
| McoGA1 | 63 | 62.7 |
| ParGA1 | 62.1 | 62.1 |
| RmiGA1 | 66.2 | 66 |
| RstGA1 | 65.1 | 64.9 |
| SfuGA2 | 64.8 | 64.6 |
| SobGA1 | 85.3 | 85.1 |
| SraGA1 | 61.6 | 61.3 |
| SraGA3 | 61.2 | 61 |
| TinGA1 | 64.5 | 64.3 |
| ZmeGA1 | 61 | 61 |

### Example 4

Constructions, Transformation and Expression of Combinatorial Library of Variant Glucoamylases in Trichoderma reesei

The DNA expression cassettes of glucoamylase variants containing combinations of selected mutations (Table 1) were obtained by using standard molecular biology techniques using the pIlc vector described above and the Seamless cloning and assembly kit (Invitrogen), according to the manufacturer's protocol.

Preparation of protoplasts and transformations were carried out as described in PCT Publication No. WO2013/102674, and the expression cassettes were inserted at specific locations in the fungal host genome. RNP complexes assembled in vitro were added to transformation mixtures as described in Example 1 that were then plated on 24 well microtiter plates with selective agar minimal medium. Once transformants grew and sporulate sufficiently, they were scraped and re-patched on a fresh selective agar plate. The transformants were further cultured and the culture supernatants were prepared as described in Example 1 to obtain protein quantified samples of the glucoamylase enzymes of interest. Protein quantification was performed as described in Example 1.

### Example 5

### Evaluation of the Enzymatic Activity of Additional Mucorales-Clade Glucoamylase Variants in Saccharification-Relevant Assays

For the activity assays, samples were normalized in 20 mM sodium acetate buffer pH 4.5 containing 0.005% (v/v) Tween-80 to a concentration of 80 ppm for the saccharification assay and reversion assay. A 10 ppm GA solution was made for dosing all colorimetric ABTS assays. Samples of the parent molecule (SEQ ID NO:34) were included in the assay plates for comparison. SEQ ID NO:34 is equivalent to SEQ ID NO:4 (SvaGA1) with Ala at position 66 and Pro at position 102.

The saccharification performance of variants and parent molecule (SEQ ID NO:34) was evaluated at pH 4.5 and 62° C. after 48 hr incubation using Maltosweet G 120 as substrate (DE=11−14) purchased from Tate & Lyle. Starting DS content of saccharification blend was 34%. The performance of the glucoamylases was tested at the dosage of 29 μg/gds. For this evaluation, the pullulanase OPTIMAX™ L 2500 was dosed at 0.454 ASPU/g DS and the alpha-amylase GC626™ was dosed at 0.088 SSU/g DS for each incubation. Reactions consisting of 70 μl of maltodextrin and 12 μl of saccharification blends (glucoamylase, alpha-amylase and pullulanase) were incubated in 384-well MTP at pH 4.5, 62° C. for 48 hr with shaking at 900 rpm. All the reactions were quenched by addition of 5 mM H₂SO₄ in a ratio of 1:33 saccharification syrup to acid solution. Quenched aliquots were analysed by HPLC for product formation using an Agilent 1260 series system with a double Phenomenex Rezex-RFQ Fast Acid H+(8%) 100×7.8 mm column (cat #00D-0223-K0) running at 80° C. for 5.3 minutes. For this purpose, 5 L sample was injected on the column and carbohydrates were separated with an isocratic gradient of 5 mM H₂SO₄ as the mobile phase at a flow rate of 1.0 mL/min. The oligosaccharide products were detected using a refractive index detector. Standards were run to determine elution times of each degree of polymerization (DP(n)) of sugars of interest (where n represents the number of saccharides, e.g., DP3+, DP3, DP2 and DP1). The total integrated peak area of the identified saccharification products was used to calculate the relative content of each (oligo)saccharide. The improved saccharification yield of the variants over the parent is reported in Table 3 as % glucose. A value of at least 0.3% increase in glucose was used as selection cut-off for mutations with positive effect on GA performance in saccharification under the given conditions.

### Specific Activity Determination Using Colorimetric Read Out

Glucoamylase variant specific activity was determined based on the release rate of glucose from selected substrates using the coupled glucose oxidase/peroxidase (GOX/HRP) and 2,2′-Azino-bis 3-ethylbenzothiazoline-6-sulfonic acid (ABTS) method (described in Anal. Biochem. 105 (1980), 389-397).

An aliquot of 10 ppm of glucoamylase culture sample was combined in a 384-well MTP with 50 μl freshly prepared reagent A solution containing 25 mM sodium acetate buffer (pH 4.5), with final concentrations 25 U/mL HRP (Sigma-Aldrich, Prod.nr: P8375), 62 U/mL GOX (Sigma-Aldrich, Prod.nr: G7141) and 0.01% Tween-80. Final enzyme concentrations in the various substrate assays were: 1.67 ppm in maltose assay, 0.43 ppm in maltodextrin assay, 1.43 ppm in panose assay, 0.72 ppm in pullulan assay and 0.32 ppm in soluble starch assay. To initiate the reaction 40 μl of substrate-containing reagent B was added consisting of 3.2 mg/mL ABTS (Sigma-Aldrich, Prod.nr: A1888), 25 mM sodium acetate buffer (pH 4.5) and 0.01% Tween-80. Final substrate concentrations of the various assays were: 4.5 mM maltose (Sigma-Aldrich, Prod.nr: 47288), 15 mM panose (Megazymes, O-PAN), 1.5% (w/v) pre-boiled pullulan (Sigma-Aldrich, Prod.nr: P4516), 0.5% (w/v) pre-boiled soluble starch (Sigma-Aldrich, Prod.nr: S9765) and 0.12% (w/v) maltodextrin (Sigma-Aldrich, Prod.nr: 31410). Reagents were mixed vigorously, and the absorbance kinetics was measured immediately at 405 nm for 3.5 minutes at 17 seconds intervals at room temperature using a plate reader from Molecular Devices. The increase of the absorbance rate at 405 nm is proportional to the hydrolytic activity of the enzyme. Reactions were run in triplicate and a linear regression was used to determine the Vo of the samples. The data generated this way was used to calculate the performance of each sample on each substrate. The performance of each variant is calculated by dividing the Vo of a sample by the Vo of the parent GA This ratio was defined as Performance Index (PI). A PI value equal to or larger than 1.1 over that of the parent GA was used as cut-off for the selection of mutations with beneficial effect on the specific activity of the molecule. Results are shown in Table 3.

### Thermostability Evaluation of Glucoamylase Variants

Glucoamylase variant protein normalized to 10 ppm were incubated in a PCR MTP at 58° C. for 30 min in a Mastercycler Pro 384 (Eppendorf). After incubation, the MTPs were cooled to room temperature and were used to determine the specific activity on maltodextrin-ABTS substrate as described above. The percent (%) residual activity (activity remaining after heat treatment) of the glucoamylase variants was determined by dividing the activity of the heat-incubated samples with that of non-incubated samples. An improved thermostability of greater than 3% above that of the parent enzyme was used as cut-off in the selection of mutations with beneficial effect on the thermostability of the molecule. Results are shown in Table 3.

### Reversion Reaction Evaluation of Glucoamylase Variants

Glucose condensation activity (reversion activity) for each GA variant was tested by combining 70 μl of 45% (w/v) glucose in 20 mM sodium acetate buffer pH 4.5 with 10 μl of enzyme sample in 384 well MTPs and incubated with shaking at 50° C. for 66 hours. The reactions were quenched by mixing 3 μl of reaction cocktail with 97 μl of 5 mM H₂SO₄. Reaction products (glucose and condensation products such as DP2, DP3 and DP3+) were determined and quantified by HPLC analysis as described above.

The performance of GA variants was compared against that of the parent molecule (SEQ ID NO:34). To determine the impact of amino acid substitutions on this GA property, a reduction in reversion yield expressed as % condensation product (DP2) was determined and the results are shown on Table 3. A value of equal or more than 0.1% decrease in DP2 is considered a positive effect on reversion under the given conditions.

Note that for reversion reactions and for saccharification yields the values are calculated as a difference in percent yields (e.g. Variant x has a 95.2% DP1 yield vs 94.8% DP1 for the parent GA, thus giving a saccharification yield improvement yield of 0.4%).

TABLE 3
Performance of multiply substituted Mucorales-clade glucoamylase variants compared to SEQ ID NO: 34 parent enzyme.

| | | | | | Reduced | Improved | | |
|---|---|---|---|---|---|---|---|---|
| | | | | | Reversion | saccharification | | Thermostability, |
| Mutations versus | | | | | over the | yield | PI | % |
| parent GA | PI | PI | PI | PI | parent GA, | over parent, | Soluble | Residual |
| (SEQ ID NO: 34) | Maltodextrin | Maltose | Panose | Pullulan | % DP2 | % glucose | starch | Activity |
| none | 1.0 | 1.0 | 1.0 | 1.0 | 0 | 0 | 1.0 | 64 |
| L141F-E281D-Q441W | | 1.1 | | | 0.2 | | | |
| D236S-E281D | | | 1.2 | | 0.2 | | | 69 |
| S215R-Q441W | | | 1.1 | | | | | |
| A143G-N321D-A434S | 1.1 | | | | 0.2 | | 1.1 | 74 |
| N321D-A434S | 1.2 | | | | 0.1 | | 1.1 | |
| A143G-A434S | 1.2 | | | | 0.2 | | 1.1 | 68 |
| S215R-D236S-E281D-Q441W | | 1.2 | 1.1 | | 0.1 | | | |
| A079C-A143G | 1.1 | | 1.1 | 1.1 | | | 1.1 | |
| N321D-K350T-A434S | 1.1 | | | | 0.2 | | | 73 |
| K350T-A434S | 1.1 | | | | 0.2 | | 1.2 | |
| S067M-N158A-E281D | | | 1.1 | | | 0.4 | | |
| F156C-Y192F-Q403K | | | | | | | | 83 |
| N351E-Q403K | | 1.1 | 1.1 | | | | | 73 |
| G052N-I084L-M140C-Y422V | | | | | 0.2 | | | 89 |
| G052N-I084L | | | | | 0.2 | | | 83 |
| T243P-F290V | | | | | 0.1 | 0.4 | | 68 |
| G052N-M140C-Y422V | | | | | 0.1 | | | 75 |
| F290V-K350E | | | 1.1 | | | 0.4 | | 67 |
| N302K-Q441W | | | | | 0.1 | | | |
| F156C-Y404K | | | | | | | | 67 |
| S067M-Y404K | | | | | | | | 69 |
| G052N-L141F | | | | | 0.2 | | | 82 |
| A066C-S119A-I164T | 1.2 | | 1.2 | | | | 1.1 | |
| G052N-N351E | | | | | 0.1 | | | 76 |
| S233M-G445Y | 1.1 | | | | | | | 69 |
| A066C-S233M-G445Y | 1.1 | | | | | | | |
| A066C-S233M | 1.2 | | | | | | 1.1 | |
| S218H-F290V | | | | | | | | 67 |
| S067M-N302H | | | | | | | | 80 |
| A066C-S119A | 1.1 | | 1.1 | | | 0.4 | 1.1 | |
| T243P-G445Y | 1.1 | | | | | | 1.1 | |
| Y192F-T243P | 1.1 | | 1.1 | | | | 1.1 | |
| F156C-T243P | 1.1 | | 1.1 | | 0.1 | | 1.1 | 72 |
| F156C-Y192F-T243P | 1.1 | | | | | | 1.1 | 76 |
| S020E-F156C-Y192F-T243P | 1.1 | | | | | | 1.4 | |
| E023L-A066C | 1.2 | | | | | | 1.1 | 76 |
| E023L-A066C-S119A | 1.1 | | | | | | 1.1 | |
| E023L-S119A | 1.1 | | | | | | 1.1 | |
| S020E-Y192F | 1.2 | | | | | | 1.2 | |
| E023M-S119A-S221R-Y404K | 1.1 | | | | | 0.4 | | |
| Y192F-N310V | 1.2 | | 1.1 | | | | 1.2 | 73 |
| E023M-N302H | | | 1.2 | | | | | 70 |
| E023M-S119A-Y404K | 1.3 | | | | | | 1.2 | |
| Y192F-F416Y | 1.1 | | 1.1 | | | | | 77 |
| S119A-N302H | | | 1.1 | | | | | 87 |
| N310V-F416Y-G445Y | | | | | | 0.3 | | 80 |
| W235Y-F416Y | | | | | | 0.5 | | 76 |
| N158A-S221R-F290V | | | | | 0.1 | 0.4 | | 70 |
| G052N-Y404K | | | | | 0.2 | | | 83 |
| E023M-F449Y | | | 1.1 | | | | | |
| N158A-V172L-S221R-F290V | | | | | | 0.5 | | |
| N158A-V172L | 1.1 | | 1.1 | | | 0.7 | | |
| V172L-F290V | | | 1.2 | | | 0.7 | | |
| E023M-G052N-Y404K | | | | | 0.2 | 0.6 | | 87 |
| E023M-R210L | | | | | | 0.4 | | |
| R210L-F449Y | | 1.1 | 1.1 | | | | 1.1 | |
| D081S-E281D | | | 1.1 | | | 0.4 | | 75 |
| T157I-E281D | | | 1.2 | | | | | 71 |
| D081S-T157I-D236S | | | 1.2 | | | | | |
| T243P-N302K-F416Y | | | 1.1 | | 0.1 | 0.7 | | 84 |
| I164T-S215R | | | 1.2 | | 0.1 | | | |
| M140C-Y422V | | | | | | | | 78 |
| M140C-Y165W-N302K-Y422V | | | | | 0.2 | | | 87 |
| S119A-N302K | | | | | 0.1 | 0.7 | | 86 |
| M140C-N302K-Y422V | | | | | | | | 82 |
| G052N-F416Y | | | | | 0.1 | | | 70 |
| G052N-F416Y-G445Y | | | | | 0.2 | 0.4 | | 90 |
| S119A-E253F-N310V-Q403K | | | | 1.2 | | 0.4 | | 81 |
| S067M-T157I-S218H-N302H- | | | | | | 0.4 | | 85 |
| F416Y | | | | | | | | |

All the SvaGa1 variants described on Table 3 have significantly improved performance when compared to parent molecule (SvaGA1+66A/102P) in at least one parameter relevant to GA performance.

### Example 6

Effect of N-Glycosylation on Variants of Saksenaea vasiformis B4078 Glucoamylase

To investigate the effect of N-glycosylation on the performance of glucoamylases SvaGA1v2 (SEQ ID NO:3) and SvaGA1v3 (SEQ ID NO:34), several variant sequences were generated using molecular biology techniques know in the art. Amino acid substitutions with the potential to generate new N-glycosylation sites were evaluated in variants corresponding to SEQ ID NO: 38, 39, 40, 41, 42, 43, 44, and 45.

The polynucleotides (codon modified sequences used as expression cassettes) encoding the GA variant sequences were synthesized by Generay (Generay Biotech Co., Ltd, Shanghai, China) and inserted into the pGX256 expression vector, a derivative vector from pTTT (see, U.S. Patent Application Publication No. 20110020899). All plasmids were transformed into a suitable Trichoderma reesei strain using protoplast transformation (Te'o et al., J. Microbiol. Methods 51:393-99, 2002). The transformants were selected and fermented by the methods described in WO 2016/138315. Supernatants from these cultures were used to confirm the Protein expression by SDS-PAGE analysis.

Fungal cell cultures were grown in a defined medium as described by Lv et al. ((2012) Plasmids 67:67-71). Clarified culture broth were collected after 96 hours by centrifugation. The glucoamylases variants were purified by methods known in the art. The column chromatography fractions containing the target Protein were pooled, concentrated and equilibrated to 20 mM sodium acetate pH 5.0, 150 mM sodium chloride using an Amicon Ultra-15 device with 10 K MWCO. The purified samples were approximately 99% pure (by SDS-PAGE analysis) and were stored in 40% glycerol at −80° C. until use.

### Thermostability Evaluation of Glucoamylases

The thermostability of the N-glycosylation variants described on Table 4 was compared with pre-incubations of the enzyme samples (20 ppm) at 55° C. for 40 min. The pre-incubation at 4° C. for 40 min was included and set as 100% activity of each glucoamylase sample. The residual activity of the glucoamylases after pre-incubation was then measured using the coupled glucose oxidase/peroxidase (GOX/HRP) and 2,2′-Azino-bis 3-ethylbenzothiazoline-6-sulfonic acid (ABTS) method (Anal. Biochem. 105 (1980), 389-397). Substrate solutions were prepared by mixing 9 mL of soluble starch (1% in water, w/w) and 1 mL of 0.5 M pH 4.5 sodium acetate buffer in a 15-mL conical tube. Coupled enzyme (GOX/HRP) solution with ABTS was prepared in 50 mM sodium acetate buffer (pH 5.0), with the final concentrations of 2.74 mg/mL ABTS, 0.1 U/mL HRP, and 1 U/mL GOX. The glucoamylase sample after pre-incubation (10 μL) was transferred into a new microtiter plate (Corning 3641) containing 90 μL of substrate solution preincubated at 50° C. for 5 min at 600 rpm. The reactions were carried out at 50° C. for 10 min with shaking (650 rpm) in a thermomixer (Eppendorf), then 10 μL of the reaction mixtures was quickly transferred to new microtiter plates (Corning 3641), respectively, followed by the addition of 90 μL of ABTS/GOX/HRP solution. Absorbance at 405 nm was immediately measured at 11 seconds intervals for 5 min using a SoftMax Pro plate reader (Molecular Device). The output of the reaction rate, Vo, was used to indicate the glucoamylase activity.

Using the method mentioned above, the residual activities of the N-glycosylation variants SvaGA1v2_D81N_K83T (SEQ ID NO:38), SvaGA1v2_A153T (SEQ ID NO:39), SvaGA1v2_S370N_A372S (SEQ ID NO:40), SvaGA1v2_D81N_K83T_A153T (SEQ ID NO:41), SvaGA1v3_D81N_K83T (SEQ ID NO:42), SvaGA1v3_A153T (SEQ ID NO:43), SvaGA1v3_S370N_A372S (SEQ ID NO:44), and SvaGA1v3_D81N_K83T_A153T (SEQ ID NO:45) were determined, compared to the respective parent sequences SvaGA1v2 (SEQ ID NO:3) and SvaGA1v3 (SEQ ID NO:34). Table 4 shows the results of thermostability comparison among the variants designed to evaluate the impact of glycosylation on the Saksenaea vasiformis B4078 glucoamylase backbone. The GA variants were pre-incubated at 55° C. for 40 min followed by residual activity measurement at 50° C. for 10 min at pH 4.5.

TABLE 4
Thermostability comparison for variants of SvaGA1 glucoamylase
when introducing additional N-glycosylation sites.

| | Activity (no | Activity | Residual |
|---|---|---|---|
| | pre- | (with pre- | activity |
| Sample information | incubation) | incubation) | (%) |
| SvaGA1v2_D81N_K83T | 113.7 | 39.0 | 34% |
| SvaGA1v2_A153T | 116.0 | 33.8 | 29% |
| SvaGA1v2_S370N_A372S | 94.9 | 25.0 | 26% |
| SvaGA1v2_D81N_K83T_ A153T | 105.1 | 34.4 | 33% |
| SvaGA1v2 | 111.8 | 24.0 | 21% |
| SvaGA1v3_D81N_K83T | 83.8 | 57.2 | 68% |
| SvaGA1v3_A153T | 79.4 | 56.8 | 72% |
| SvaGA1v3_S370N_A372S | 76.2 | 51.8 | 68% |
| SvaGA1v3_D81N_K83T_A153T | 80.2 | 60.4 | 75% |
| SvaGA1v3 | 83.5 | 54.9 | 66% |

As shown in Table 4, the N-glycosylation variants showed higher residual activity than their parent molecule, especially the variants with substitutions D81N_K83T_A153T. SvaGA1v2_D81N_K83T_A153T retained 33% of its activity while SvaGA1V2 parent retained 21%. SvaGA1v3_D81N_K83T_A153T outperformed its parent molecule, with 75% residual activity maintained versus 66% for the parent SvaGA1v3.

### Example 7

### Structural Elements of Mucorales-Clade Glucoamylase and Variants

A substitution at the Asn glycosylation site at position 75 was observed to lead to a reduction in catalytic activity. The variant SvaGA1v2 N75D shows an approximate 50% reduction in activity when tested at 55° C. for 40 min as compared to parent SvaGA1v2 protein. At the time of submission, no existing glucoamylase structure in the Protein Data Bank (worldwideweb.rcsb.org) contained an N-glycosylation site at Asn75 or the structurally equivalent position. This glycosylation site is focused in the Mucorales clade of glucoamylases described in Example 3. To identify additional glucoamylases with these stabilizing interactions, the nr database of non-redundant protein sequences (National Center for Biotechnology Information) was searched with the SvaGA1v2 (SEQ ID NO:3) sequence as a query using all default BLAST parameters. The top 500 sequences, containing sequences from various fungal and bacterial phyla, were aligned with MOE 2019.0102 (Chemical Computing Group, Montreal, Canada). Sequences containing the N-glycosylation motif N-X-(S/T) (Schwarz & Aebi, (2011) Current Opinion in Structural Biology. 21: 576-582.) at the structurally equivalent position to Asn94 in SvaGA1v2 (SEQ ID NO:3) were identified. The resulting source organisms included 50 species of Mucoromycota fungi and only one other type of organism, a single bacterial species.

To understand the structural basis of the activity improvement from glycosylation at Asn 75, a crystallographic structure was determined. A variant of the glucoamylase from Saksenaea vasiformis (SvaGA1v2) (SEQ ID NO:3) was cloned and expressed using standard methods, as described in PCT Application Publication Nos. WO2011/063308 and WO2016/138315. The fermentation broth was concentrated with a VivaFlow 200 ultrafiltration device (Sartorius Stedim, Goettingen, Germany). After adding (NH₄)₂SO₄ to a final concentration of 1 M, the concentrates were loaded onto a HiPrep™ Phenyl FF 16/10 column (GE Healthcare, Pittsburgh, USA) pre-equilibrated with 20 mM sodium phosphate buffer (pH 7.0, supplemented with 1 M (NH₄)₂SO₄) (Buffer A). After sample loading, the column was washed with Buffer A until the UV (A280) baseline became stable. The protein elution was started with a gradient from 100% Buffer A to 100% 20 mM sodium phosphate (pH 7.0) (Buffer B), for 20 min at a flow rate of 5 mL/min. Fractions containing the target protein were pooled, concentrated, and buffer exchanged into Buffer B via the 10 kDa Amicon Ultra-15 device (Merck Millipore Ltd., Darmstadt, Germany).

Crystals were grown by the hanging drop method using a reservoir solution of 2.13 M ammonium sulfate, 0.1 M sodium acetate trihydrate, pH 4.3. The drop contained 2 μL of 11.5 mg/mL protein and 2 μL of reservoir solution. Cryo-protection included 17% total glycerol in reservoir solution.

Native diffraction data were collected at Stanford Synchrotron Radiation Lightsource (SSRL) on beamline 9-2 using the BLU-ICE data collection environment (Hypertext Transfer Protocol Secure://pubmed.ncbi.nlm.nih.gov/12409628/), performed by Accelero Biostructures, Inc., California. The data sets were collected at 100 K, using a Pilatus 6M detector (Dectris AG, Switzerland). The data were processed to 0.95 Å resolution using XDS (Hypertext Transfer Protocol Secure://pubmed.ncbi.nlm.nih.gov/20124692/).

Molecular replacement was carried out with Phaser (McCoy et al., J. Appl. Cryst. (2007). 40, 658-674) using a homology model as a search model. The homology model was constructed with MOE (Chemical Computing Group, Montreal, Canada) using default parameters with the glucoamylase from Saccharomycopsis fibuligera, PDB code 2FBA. (Sevcik, et al., (2006) FEBS J 273: 2161-2171). Nineteen rounds of rebuilding and refinement with REFMAC 5.8 (Vagin, et al., (2004) Acta Crystallogr. D60: 2284-2295) and Coot (Emsley & Cowtan (2004). Acta Crystallogr. D60, 2126-2132) were performed with isotropic temperature factors, then 3 final rounds of refinement were performed with anisotropic temperature factors. The resolution range for refinement was 0.97 Å to 40.70 Å. After final refinement using anisotropic temperature factors, Rwork=0.12 and Rfree=0.14. In the highest resolution shell (0.97 Å to 0.995 Å), Rwork=0.29 and Rfree=0.31.

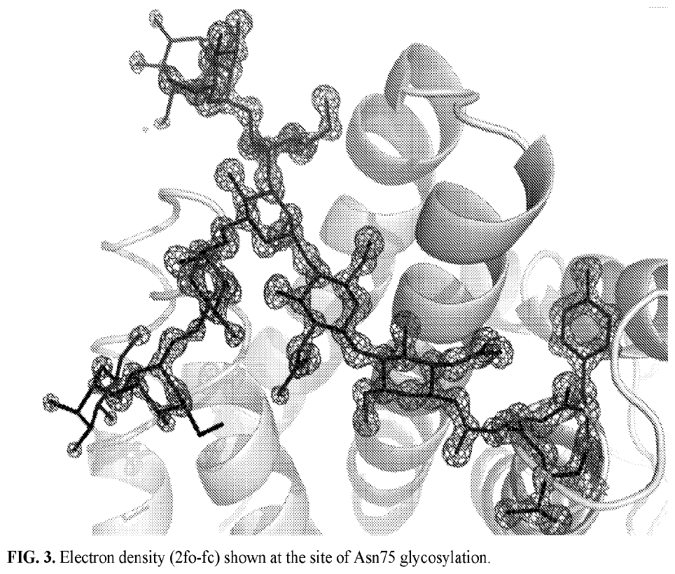

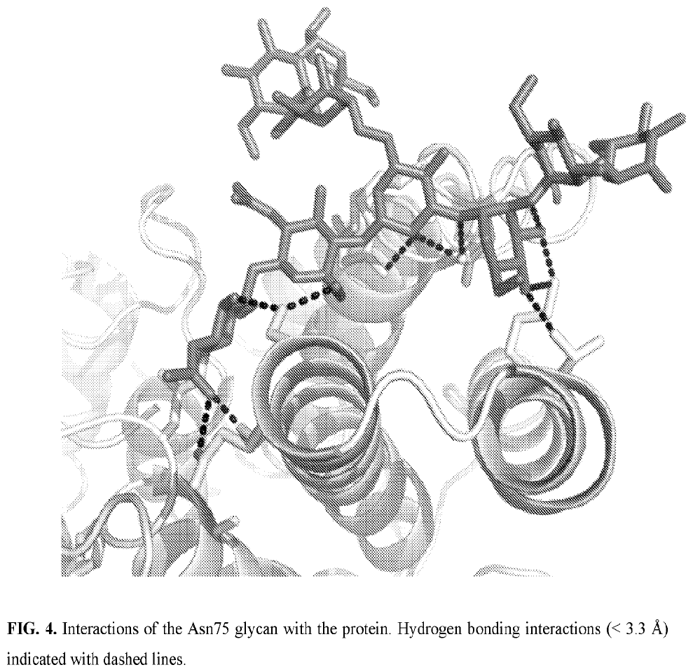

The x-ray diffraction structure determined for SvaGA1v2 (SEQ ID NO: 3) showed clear electron density for the N-glycosylation site at Asn75. At the time of submission, no existing glucoamylase structure in the Protein Data Bank (worldwideweb.rcsb.org) contained an N-glycosylation site at Asn75 or the structurally equivalent position. Electron density for the Asn75 glycan is shown in FIG. 3. The structure shows an extensive network of hydrogen bonding contacts of the glycan to the protein, bridging four helices at the protein surface (FIG. 4). Table 5 identifies atoms and contact distances. Without being constrained to a theory, it is proposed that this extensive hydrogen bonding network between the Asn75 glycan and the protein chain stabilizes these four protein helices, which in turn provide a structural anchor for the substrate binding site and the interactions that stabilize and position the substrate within the catalytic active site.

TABLE 5
Hydrogen bonding contact distances
between protein and Asn75 glycan atoms

| Region | Residue | Å |
|---|---|---|
| Helix around 130-150 | Gln149 NE2-NAG 1 O7 | 2.9 |
| | Ser148 OG-NAG 1 O3 | 2.7 |
| | Ser148 OG-NAG 2 O6 | 2.7 |
| Helix around 153-172 | Thr157 OG1-MAN 4 O4 | 2.8 |
| | Lys 161 NZ-MAN 4 O4 | 3.2 |
| | Lys 161 NZ-MAN 4 O3 | 3.1 |
| Helix around 192-211 | Asn211 OD1-BMA 3 O3 | 3.1 |
| | Asn211 ND2-BMA 3 O2 | 2.7 |
| | Asn211 O-MAN 4 O3 | 2.7 |
| | Arg210 NH1-BMA 3 O2 | 2.9 |

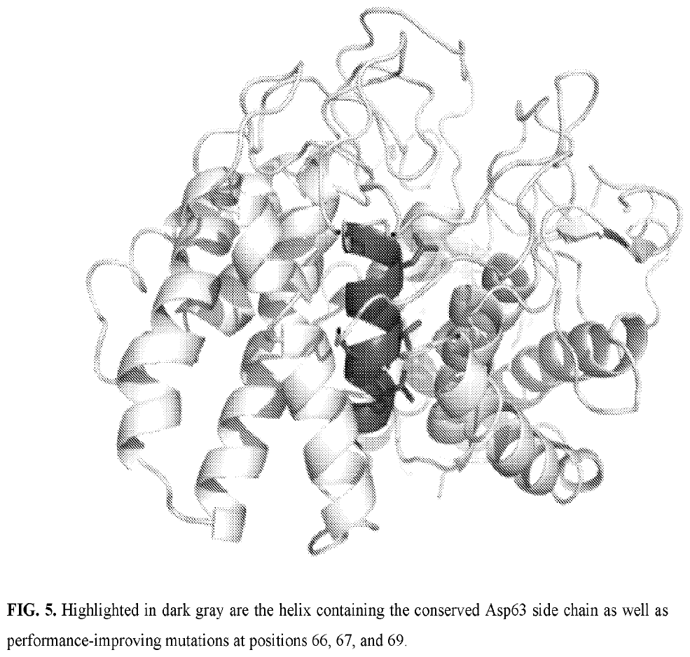

The structure of SvaGA1v2 (SEQ ID NO: 3) also suggests rationale for increases in performance of enzyme variants as described in Examples 2 and 5. In particular, a helix spanning residues 60-72 contains the conserved active site residue Asp62 which is expected to stabilize developing positive charge in the catalytic cycle in a well-studied glucoamylase (Aleshin et al., (1996) Biochemistry 35: 8319-8328). Mutations to residues of this helix, including at positions 66, 67, and 69, are shown in Examples 2 and 5 to improve application performance. Without being bound to theory, these mutations may alter the position of the helix in the active site and therefore adjust the precise placement of Asp62 for improved stabilization of developing positive charge. The helix, the conserved Asp62 side chain, and the sites of performance-improving mutations are shown in FIG. 5.

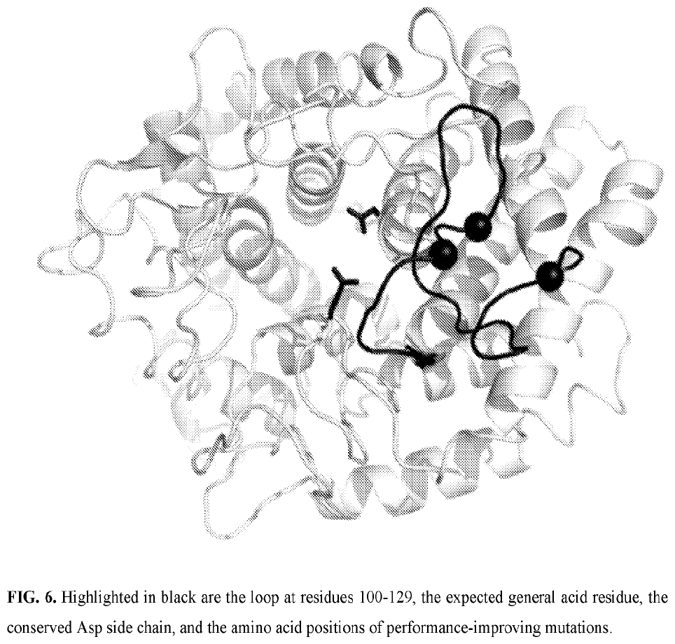

Additional performance-improving mutations are located on a substrate-binding loop comprising residues 100-129. Mutations in position 102, 119, and 121 in particular are shown in Examples 2 and 5 to improve performance of the enzyme. The loop that these residues lie on, residues 100-129, forms a significant portion of the surface area of the active site. The loop is directly adjacent to the helix that carries the conserved Asp62 side chain, and it further makes hydrogen bonding contacts to Glu184, the expected general acid in the glucoamylase catalytic mechanism. Without being constrained to a theory, changes in the conformation of the loop caused by mutations in these positions may lead to alterations in the precise positioning of the general acid relative to the substrate that improve application performance. FIG. 6 shows the position of the loop, the expected general acid residue, and the positions of performance-improving mutations.

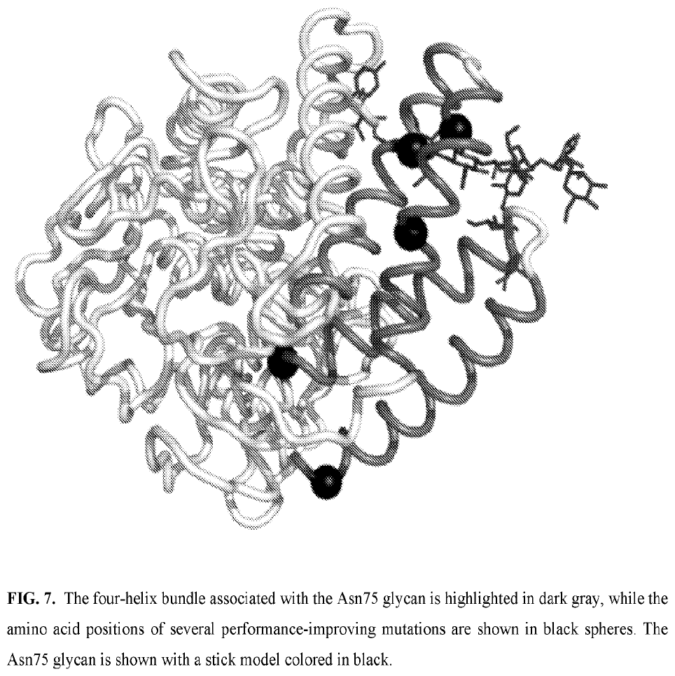

Another set of performance-improving mutations lie on the bundle of four helices contacted by the Asn75 glycan. Mutations in positions 143, 156, 164, 192, and 233 led to increased performance, as shown in Example 5. These mutations are shown in FIG. 7, with the four-helix bundle associated with the glycan chain of the Asn75 residue highlighted in dark gray. The four-helix bundle provides a solid structural core which anchors the substrate binding loops in the active site.

What is claimed is:

1. A glucoamylase variant or fragment thereof comprising one or more amino acid substitutions at residue positions corresponding to positions 20, 21, 23, 37, 49, 51, 52, 66, 67, 69, 73, 77, 79, 80, 81, 84, 92, 94, 102, 119, 121, 134, 140, 141, 143, 156, 157, 158, 164, 165, 166, 172, 192, 203, 210, 213, 214, 215, 218, 221, 222, 233, 235, 236, 238, 243, 252, 253, 274, 278, 281, 290, 302, 310, 321, 338, 341, 350, 351, 352, 354, 370, 390, 403, 404, 405, 416, 418, 422, 430, 434, 440, 441, 444, 445, and/or 449 of SEQ ID NO:4 and/or an equivalent position in a parent glucoamylase.

2. The glucoamylase variant of claim 1, wherein the equivalent position is determined by sequence identity and said parent glucoamylase has at least 80% sequence identity and less than 100% sequence identity with SEQ ID NOs:2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37.

3. The glucoamylase variant of claim 2, wherein the equivalent position is determined by sequence identity and wherein the parent glucoamylase comprises SEQ ID NOs: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37.

4. The glucoamylase variant of any one of claims 1-3, wherein the parent glucoamylase is a Mucorales-clade glucoamylase.

5. The glucoamylase variant of any one of claims 1-4, wherein said one or more amino acid substitutions comprise one or more of X020A/E/F/G/P; X021M/S/T/W; X023L/M; X037C/N; X049W; X051K/L/V/Y; X052F/N/P; X66A/C/F/M/P/T/W; X067A/C/M; X069A/C/K; X073N/P; X077M/P/S; X079C/R/T; X080H/K/N; X081N/S; X084L/V; X092C/I/M; X094A/G/Y; X102G; X119A/D/G; X121G/L/M/P/V; X134Q/S/W; X140C/I/Q; X141F/K; X143G; X156C/P; X157A/I; X158A/Y; X164L/T; X165I/T/W; X166A/F/G/H/R; X172G/L; X192F/R; X203C/M/Q/W/Y; X210A/F/G/I/L/M/N/W; X213H/R; X214A/C/E/G/L/T/Y; X215C/D/F/H/R/V/W; X218F/A/H/K/Q/V/Y; X221M/R/T; X222C/M/V; X233M/PT/Y; X235F/Y; X236A/Q/S; G238H/M/N/S/T/V; X243A/P/S; X252F/H/M/T; X253K/V; X274A/D/K; X278A; X281D/P; F290M/V; X302F/H/K/M/P/Q/S/T/V/W; X310T/V/Y; X321D; X338I; X341M/T; X350T/C/E/I; X351E/V; X352D/N/S; X354L/M; X370M/N/R; X390D/E/L; X403G/K/R; X404K/M; X405Q/S/Y; X416C/Y; X418E/W; X422A/F/V; X430A/Q; X434S; X440G/H/L; X441L/S/W; X444L/P/S; X445M/Y; and/or X449L, where X is any amino acid corresponding to the equivalent position in SEQ ID NO:4 and/or an equivalent position in a parent glucoamylase or fragment thereof.

6. The glucoamylase variant of claim 5, wherein said one or more amino acid substitutions comprise one or more of S020A/E/F/G/P; K021M/S/T/W; E023L/M; E037C; S049W; A051K/L/V/Y; G052F/N/P; X66A/C/F/M/P/T/W; S067A/C/M; V069A/C/K; K073N/P; T077M/P/S; A079C/R/T; G080H/K/N; D081N/S; 1084L/V; V092C/I/M; F094A/G/Y; X102G; S119A/D/G; T121G/L/M/P/V; E134Q/S/W; M140C/I/Q; L141F/K; A143G; F156C/P; T157A/I; N158A/Y; 1164L/T; Y165I/T/W; K166A/F/G/H/R; V172G/L; Y192F/R; D203C/M/Q/W/Y; R210A/F/G/I/L/M/N/W; D213H/R; N214A/C/E/G/L/Y; S215C/D/F/H/R/V/W; A218F/A/H/K/Q/V/Y; S221M/R/T; G222C/M/V; S233M/PT/Y; W235F/Y; D236A/Q/S; G238H/M/N/S/T/V; T243A/P/S; V252F/H; E253K; G274A/D/K; P278A; E281D/P; F290M/V; N302F/H/K/M/P/Q/S/T/V/W; N310T/V/Y; N321D; F338I; L341M/T; K350C/E/I; N351E/V; T352D/N/S; V354L/M; S370M/N/R; S390D/E/L; Q403G/K/R; Y404K/M; H405Q/S/Y; F416C/Y; R418E/W; Y422A/F/V; T430A/Q; A434S; A440G/H/L; Q441L/S/W; A444L/P/S; G445M/Y; and/or F449L.

7. The glucoamylase variant of any one of claims 1-6, wherein said variant comprises one or more amino acid substitutions at residue positions corresponding to positions 20, 21, 51, 79, 80, 92, 102, 121, 140, 143, 157, 158, 165, 166, 192, 203, 210, 213, 214, 215, 221, 222, 233, 235, 236, 238, 243, 252, 274, 278, 281, 290, 302, 310, 321, 341, 350, 352, 354, 370, 390, 403, 404, 405, 418, 440, 441, and/or 444 of SEQ ID NO:4 and/or an equivalent position in a parent glucoamylase, and wherein said variant glucoamylase exhibits improved hydrolysis of maltose compared to a parent glucoamylase or fragment thereof lacking one or more of these substitutions.

8. The glucoamylase variant of claim 7, wherein said one or more amino acid substitutions comprise one or more of X020A/P; X021S; X051L/V; X079C/T; X080K; X092M; X102G; X121G/P/M; X140I/C; X140C; X143G, X157I; X158A; X165W; X166G/R/F; X192F; X203W; X210L; X213R; X214A/E; X215D/C/F; X221R/T/M; X222M/C; X233M/Y/P; X235Y; X236A/Q; X238S/H; X243E/V; X252C/F; X274A; X278A; X281D; X290M/V; X302K/V/P/S/W; X310T/Y; X321D, X341M; X350I/T; X352D; X354L/M; X370M; X390E; X403G/R; X404K; X405Q/S/Y; X418W/E; X440G/H; X441S; and/or X444L, where X is any amino acid corresponding to the equivalent position in SEQ ID NO:4 and/or an equivalent position in a parent glucoamylase or fragment thereof.

9. The glucoamylase variant of claim 8, wherein said one or more amino acid substitutions comprise one or more of S020A/P; K021S; A051L/V; A079C/T; G080K; V092M; X102G; T121G/P/M; M140I/C; M140C; A143G; T157I; N158A; Y165W; K166G/R/F; Y192F; D203W; R210L; D213R; N214A/E; S215D/C/F; S221R/T/M; G222M/C; S233M/Y/P; W235Y; D236A/Q; G238S/H; T243E/V; V252C/F; G274A; P278A; E281D; F290M/V; N302K/V/P/S/W; N310T/Y; N321D; L341M; K350I/T; T352D; V354L/M; S370M; S390E; Q403G/R; Y404K; H405Q/S/Y; R418W/E; A440G/H; Q441S; and/or A444L.

10. The glucoamylase variant of any one of claims 1-6, wherein said variant comprises one or more amino acid substitutions at residue positions corresponding to positions 20, 21, 23, 37, 51, 52, 67, 69, 73, 77, 79, 80, 81, 84, 92, 94, 102, 119, 121, 140, 141, 143, 164, 165, 166, 172, 210, 213, 214, 215, 218, 221, 222, 233, 236, 238, 252, 253, 274, 281, 290, 302, 321, 341, 350, 351, 352, 370, 390, 403, 404, 405, 418, 430, 440, 444, and/or 445 of SEQ ID NO:4 and/or an equivalent position in a parent glucoamylase, and wherein said variant glucoamylase exhibits improved hydrolysis of panose compared to a parent glucoamylase or fragment thereof lacking one or more of these substitutions.

11. The glucoamylase variant of claim 10, wherein said one or more amino acid substitutions comprise one or more of X020P/A; X021T/W/M; X023M/L; X037C; X051L/V; X052N; X067C; X069C/A; X073N; X077S; X079C, X080H/K; X081N; X084V/L; X092C; X094Y; X102G; X119G; X121M/L/G/P; X140C/Q/I; X141F; X143G, X164L; X165W/T; X166R; X172G; X210L; X213H; X214A/E; X215D; X218Q/A/V/Y; X221R; X222M/V/C; X233M/T/P; X236Q; X238H; X252F; X274D/K; X281D; X290M/V; X302S/K/W/Q/V; X321D, X341M/T; X350C/T; X351E; X352D/N; X370M; X390D/L/E; X403G; X403R/K; X404M/K; X405S/Q; X418E/W; X430A; X440G; X444P; and/or X445Y/M where X is any amino acid corresponding to the equivalent position in SEQ ID NO:4 and/or an equivalent position in a parent glucoamylase or fragment thereof.

12. The glucoamylase variant of claim 11, wherein said one or more amino acid substitutions comprise one or more of S020P/A; K021T/W/M; E023M/L; E037C; A051L/V; G052N; S067C; V069C/A; K073N; T077S; A079C; G080H/K; D081N; 1084V/L; V092C; F094Y; X102G; S119G; T121M/L/G/P; M140C/Q/I; L141F; A143G; 1164L; Y165W/T; K166R; V172G; R210L; D213H; N214A/E; S215D; S218Q/A/V/Y; S221R; G222M/V/C; S233M/T/P; D236Q; G238H; V252F; G274D/K; E281D; F290M/V; N302S/K/W/Q/V; N321D; L341M/T; K350C/T; N351E; T352D/N; S370M; S390D/L/E; Q403G; Q403R/K; Y404M/K; H405S/Q; R418E/W; T430A; A440G; A444P; and/or G445Y/M.

13. The glucoamylase variant of any one of claims 1-6, wherein said variant comprises one or more amino acid substitutions at residue positions corresponding to positions 20, 51, 69, 73, 77, 79, 80, 81, 84, 94, 119, 121, 134, 140, 141, 143, 156, 158, 165, 166, 203, 210, 214, 215, 218, 221, 222, 233, 235, 236, 252, 253, 274, 278, 281, 290, 302, 310, 321, 341, 350, 352, 354, 370, 390, 416, 418, 434, 440, and/or 445, of SEQ ID NO:4 and/or an equivalent position in a parent glucoamylase, and wherein said variant glucoamylase exhibits improved hydrolysis of pullulan compared to a parent glucoamylase or fragment thereof lacking one or more of these substitutions.

14. The glucoamylase variant of claim 13, wherein said one or more amino acid substitutions comprise one or more of X020F/A/G; X051K; X069C; X073P/N; X077M/P; X079C/T; X080K; X081N; X084V/L; X094G/A; X119G/D; X121L/M; X134S; X140I/C/Q; X141F; X143G; X156P/C; X158A; X165T; X166H/A/F; X203C/Q/W/Y; X210L/M/F/N/A/I; X214A/Y/L/C; X215V/C/W/D/H; X218V/K/A/Q; X221R; X222M; X233M/P; X235Y; X236Q; X252F/H; X253K; X274K/D; X278A; X281D; X290M; X302P/S/V; X310T; X321D; X341M; X350I/T; X352D/S; X354L; X370M; X390D/L/E; X404K; X416Y; X418E/W; X434S; X440G; and/or X445M, where X is any amino acid corresponding to the equivalent position in SEQ ID NO:4 and/or an equivalent position in a parent glucoamylase or fragment thereof.

15. The glucoamylase variant of claim 14, wherein said one or more amino acid substitutions comprise one or more of S020F/A/G; A051K; V069C; K073P/N; T077M/P; A079T; G080K; D081N; 1084V/L; F094G/A; S119G/D; T121L/M; E134S; M140I/C/Q; L141F; A143G; F156P/C; N158A; Y165T; K166H/A/F; D203C/Q/W/Y; R210L/M/F/N/A/I; N214A/Y/L/C; S215V/C/W/D/H; S218V/K/A/Q; S221R; G222M; S233M/P; W235Y; D236Q; V252F/H; E253K; G274K/D; P278A; E281D; F290M; N302P/S/V; N310T; N321D; L341M; K350I/T; T352D/S; V354L; S370M; S390D/L/E; Y404K; F416Y; R418E/W; A434S; A440G; and/or G445M.

16. The glucoamylase variant of any one of claims 1-6, wherein said variant comprises one or more amino acid substitutions at residue positions corresponding to positions 21, 23, 37, 52, 66, 69, 73, 77, 79, 80, 81, 84, 92, 119, 121, 134, 140, 141, 157, 158, 164, 165, 210, 213, 214, 218, 221, 222, 233, 235, 236, 243, 252, 253, 274, 281, 290, 302, 341, 350, 351, 352, 370, 390, 403, 404, 405, 416, 418, 422, 440, 441, 444, and/or 445 of SEQ ID NO:4 and/or an equivalent position in a parent glucoamylase or fragment thereof, and wherein said variant glucoamylase exhibits a higher performance index (PI) for the saccharification of soluble starch compared to a parent glucoamylase lacking one or more of these substitutions.

17. The glucoamylase variant of claim 16, wherein said one or more amino acid substitutions comprise one or more of X021T; X023M/L; X037C; X052N/F; X066A/F/T; X069C; X073N/P; X077P/M/S; X079R; X080N/H/K; X081N/S; X084L/V; X092I; X119A/G; X121M/L; X134S/Q; X140C/I/Q; X141F/K; X157A; X158A; X164L/T; X165W/T; X210W/G; X213H; X214G/Y; X218Q/F/A; X221R; X222M/V/C; X233M/P; X235F; X236Q; X243A; X252F; X253K; X274D/K/A; X281D; X290M/V; X302W/S/Q/K; X341M/T; X350C/E; X351E; X352D/N; X370M; X390D/L; X403G/R/K; X404I/M/K; X405S; X416C/Y; X418E/W; X422A; X440G; X441L; X444S/P; and/or X445M, where X is any amino acid corresponding to the equivalent position in SEQ ID NO:4 and/or an equivalent position in a parent glucoamylase or fragment thereof.

18. The glucoamylase variant of claim 17, wherein said one or more amino acid substitutions comprise one or more of K021T; E023M/L; E037C; G052N/F; V066A/F/T; V069C; K073N/P; T077P/M/S; A079R; G080N/H/K; D081N/S; 1084L/V; V092I; S119A/G; T121M/L; E134S/Q; M140C/I/Q; L141F/K; T157A; N158A; 1164L/T; Y165W/T; R210W/G; D213H; N214G/Y; S218Q/F/A; S221R; G222M/V/C; S233M/P; W235F; D236Q; T243A; V252F; E253K; G274D/K/A; E281D; F290M/V; N302W/S/Q/K; L341M/T; K350C/E; N351E; T352D/N; S370M; S390D/L; Q403G/R/K; Y404M/K; H405S; F416C/Y; R418E/W; Y422A; A440G; Q441L; A444S/P; and/or G445M.

19. The glucoamylase variant of any one of claims 1-6, wherein said variant comprises one or more amino acid substitutions at residue positions corresponding to positions 20, 21, 51, 67, 69, 73, 77, 79, 80, 81, 84, 102, 119, 121, 134, 140, 141, 143, 156, 157, 158, 165, 166, 172, 192, 203, 210, 213, 214, 215, 218, 221, 222, 233, 235, 236, 252, 274, 281, 290, 302, 310, 321, 341, 350, 351, 352, 354, 370, 390, 403, 404, 405, 418, 422, 434, 440, 444, and/or 445 of SEQ ID NO:4 and/or an equivalent position in a parent glucoamylase, and wherein said variant glucoamylase exhibits improved hydrolysis of maltodextrin compared to a parent glucoamylase or fragment thereof lacking one or more of these substitutions.

20. The glucoamylase variant of claim 19, wherein said one or more amino acid substitutions comprise one or more of X020P/A/E; X021S/M; X051V/L/K/Y; X067A; X069C; X073P/N; X077P/M; X079C/T; X080K/H; X081N; X084L/V; X102G; X119G; X121M/G/V/P; X134S/Q; X140C/I; X141F; X143G; X156P/C; X157I; X158A; X165W/T; X166G; X172G; X192F; X203W/C/M; X210L/M; X213R; X214A/E; X215D/C/F; X218V/Y/Q; X221R/T; X222M/C; X233M/P/Y; X235Y; X236Q/A; X252F; X274K/A/D; X281D; X290M/V; X302P/K/S/V/Q/W; X310T/Y; X341M; X350I/T; X351V; X352D; X354L/M; X370M; X390E/L/D; X403K/R/G; X404K/M; X405Q/S/Y; X418W/E; X422A; X434S; X440G/H; X444P; and/or X445M, where X is any amino acid corresponding to the equivalent position in SEQ ID NO:4 and/or an equivalent position in a parent glucoamylase or fragment thereof.

21. The glucoamylase variant of claim 20, wherein said one or more amino acid substitutions comprise one or more of S020P/A/E; K021S/M; A051V/L/K/Y; S067A; V069C; K073P/N; T077P/M; A079C/T; G080K/H; D081N; 1084L/V; P102G; S119G; T121M/G/V/P; E134S/Q; M140C/I; L141F; A143G; F156P/C; T157I; N158A; Y165W/T; K166G; V172G; Y192F; D203W/C/M; R210L/M; D213R; N214A/E; S215D/C/F; S218V/Y/Q; S221R/T; G222M/C; S233M/P/Y; W235Y; D236Q/A; V252F; G274K/A/D; E281D; F290M/V; N302P/K/S/V/Q/W; N310T/Y; N321D; L341M; K350I/T; N351V; T352D; V354L/M; S370M; S390E/L/D; Q403K/R/G; Y404K/M; H405Q/S/Y; R418W/E; Y422A; A434S; A440G/H; A444P; and/or G445M.

22. The glucoamylase variant of any one of claims 1-6, wherein said variant comprises one or more amino acid substitutions at residue positions corresponding to positions 23, 51, 52, 66, 67, 77, 119, 121, 134, 140, 141, 156, 157, 165, 192, 213, 221, 222, 233, 236, 252, 281, 290, 302, 350, 352, 370, 390, 403, 404, 416, and/or 422 of SEQ ID NO:4 and/or an equivalent position in a parent glucoamylase, and wherein said variant glucoamylase exhibits improved thermostability compared to a parent glucoamylase or fragment thereof lacking one or more of these substitutions.

23. The glucoamylase variant of claim 22, wherein said one or more amino acid substitutions comprise one or more of X023M; X051Y; X052N; X066A/C/F/M/W; X067C; X077P; X119A; X121M; X134S; X140I/C; X141F; X156C; X157I; X165W/I; X192F; X213R; X221R; X222M; X233M; X236Q; X252F; X281D; X290V; X302H/Q/W/S/K; X350E/C; X352D; X370M; X390L/D; X403R; X404K/M; X416Y; and/or X422A/V, where X is any amino acid corresponding to the equivalent position in SEQ ID NO:4 and/or an equivalent position in a parent glucoamylase or fragment thereof.

24. The glucoamylase variant of claim 23, wherein said one or more amino acid substitutions comprise one or more of E023M; A051Y; G052N; V066A/C/F/M/W; S067C; T077P; S119A; T121M; E134S; M140I/C; L141F; F156C; T157I; Y165W/I; Y192F; D213R; S221R; G222M; S233M; D236Q; V252F; E253F; E281D; F290V; N302H/Q/W/S/K; K350E/C; T352D; S370M; S390L/D; Q403R; Y404K/M; F416Y; and/or Y422A/V.

25. The glucoamylase variant of any one of claims 1-6, wherein said variant comprises one or more amino acid substitutions at residue positions corresponding to positions 49, 51, 52, 66, 69, 77, 80, 94, 119, 134, 158, 164, 165, 172, 192, 213, 215, 218, 222, 233, 235, 236, 238, 243, 253, 274, 302, 338, 403, 405, 416, 418, 422, 430, 440, 441, 444, 445, and/or 449 of SEQ ID NO:4 and/or an equivalent position in a parent glucoamylase, and wherein said variant glucoamylase exhibits less reversion to saccharides having a degree of polymerization (DP) greater than or equal to 2 compared to a parent glucoamylase or fragment thereof lacking one or more of these substitutions.

26. The glucoamylase variant of claim 25, wherein said one or more amino acid substitutions comprise one or more of X049W; X051Y; X052P; X66P; X069K/C; X077P/M; X080H; X094A/G; X119D; X134W/Q; X158Y; X164T/L; X165W/I; X172G; X192R/F; X213H; X215R; X218K; X222C; X233T/P; X235F; X236A; X238H/S/N; X243P/S; X253K; X274K/A; X302H/F/P/M/Q/T; X338I; X403G; X405Q/S; X416C/Y; X416Y; X418W/E; X422A; X430A/Q; X440L/H; X441W/L; X444L/P; X445Y; and/or X449L, where X is any amino acid corresponding to the equivalent position in SEQ ID NO:4 and/or an equivalent position in a parent glucoamylase or fragment thereof.

27. The glucoamylase variant of claim 26, wherein said one or more amino acid substitutions comprise one or more of S049W; A051Y; G052P; V66P; V069K/C; T077P/M; G080H; F094A/G; S119D; E134W/Q; N158Y; 1164T/L; Y165W/I; V172G; Y192R/F; D213H; S215R; S218K; G222C; S233T/P; W235F; D236A; G238H/S/N; T243P/S; E253K; G274K/A; N302H/F/P/M/Q/T; F338I; Q403G; H405Q/S; F416C/Y; F416Y; R418W/E; Y422A; T430A/Q; A440L/H; Q441W/L; A444L/P; G445Y; and/or F449L.

28. The glucoamylase variant of any one of claims 1-6, wherein said variant comprises one or more amino acid substitutions at residue positions corresponding to positions 21, 23, 51, 52, 66, 67, 79, 81, 92, 119, 140, 158, 164, 172, 192, 210, 213, 214, 215, 218, 221, 222, 233, 235, 236, 238, 243, 253, 281, 290, 302, 310, 351, 352, 354, 370, 403, 416, 422, 430, 441, and/or 445 of SEQ ID NO:4 and/or an equivalent position in a parent glucoamylase, and wherein said variant glucoamylase exhibits improved saccharification yield of glucose compared to a parent glucoamylase lacking one or more of these substitutions.

29. The glucoamylase variant of claim 28, wherein said one or more amino acid substitutions comprise one or more of X021S/W; X023L; X051Y/K; X052N; X066/A/C/F/M/W; X067M/C/A; X079R; X081S; X092C/M; X119A; X140I; X158A/Y; X164L; X172L/G; X192F; X210W; X213H; X214Y; X215F/C/R; X218H/K/A; X221M/T; X222V; X233T; X235Y/F; X236S/A; X238T/V/M; X243A; X253K; X281P/D; X290V; X302K/Q/H/W; X310V/T; X351V; X352S; X354L; X370R; X403K; X416Y; X422F; X430A; X441L; and/or X445Y, where X is any amino acid corresponding to the equivalent position in SEQ ID NO:4 and/or an equivalent position in a parent glucoamylase.

30. The glucoamylase variant of claim 29, wherein said one or more amino acid substitutions comprise one or more of K021S/W; E023L; A051Y/K; G052N; V066/A/C/F/M/W; S067M/C/A; A079R; D081S; V092C/M; S119A; M140I; N158A/Y; I164L; V172L/G; Y192F; R210W; D213H; N214Y; S215F/C/R; S218H/K/A; S221M/T; G222V; S233T; W235Y/F; D236S/A; G238T/V/M; T243A; E253K; E281P/D; F290V; N302K/Q/H/W; N310V/T; N351V; T352S; V354L; S370R; Q403K; F416Y; Y422F; T430A; Q441L; and/or G445Y.

31. The glucoamylase variant of any one of claims 1-29, wherein said variant comprises one or more amino acid substitutions at residue positions corresponding to positions 20, 21, 23, 37, 51, 52, 66, 67, 69, 73, 77, 79, 80, 81, 84, 92, 94, 102, 119, 121, 134, 140, 141, 156, 157, 158, 164, 165, 166, 172, 192, 203, 210, 213, 214, 215, 218, 221, 222, 233, 235, 236, 238, 243, 252, 253, 274, 278, 281, 290, 302, 310, 341, 350, 351, 352, 354, 370, 390, 403, 404, 405, 416, 418, 422, 430, 440, 441, 444, and/or 445, of SEQ ID NO:4 and/or an equivalent position in a parent glucoamylase or fragment thereof, and wherein said variant glucoamylase exhibits two or more of i) improved hydrolysis of maltose; ii) improved hydrolysis of panose; iii) improved hydrolysis of pullulan; iv) improved hydrolysis of soluble starch; v) improved hydrolysis of maltodextrin; vi) improved thermostability; vii) less reversion to saccharides having a DP greater than or equal to 2; and/or viii) improved saccharification yield of glucose compared to a parent glucoamylase lacking one or more of these substitutions.

32. The glucoamylase variant of claim 31, wherein said one or more amino acid substitutions comprise one or more of X020A/P; X021M/S/T/W; X023L/M; X037C; X051K/L/V/Y; X052N; X066A/C/F/M/W; X067A/C; X069C; X073N/P; X077M/P/S; X079R/T; X080H/K; X081N/S; X084L/V; X092C/M; X094A/G; X102G; X119A/D/G; X121G/L/M/P; X134Q/S; X140C/I/Q; X141F; X156C/P; X157I; X158A/Y; X164L/T; X165I/T/W; X166G/F/R; X172G; X192F; X203C/W; X210L/M/W; X213H/R; X214A/E/Y; X215C/D/F/R; X218A/K/Q/V/Y; X221M/R/T; X222C/M/V; X233M/P/T/Y; X235F/Y; X236A/Q; X238H/S; X243A; X252F; X253K; X274A/D/K; X278A; X281D; X290M/V; X290V; X302H/K/P/Q/S/V/W; X310T/Y; X341M/T; X350C/E/I; X351E/V; X352D/N/S; X354L/M; X370M; X390D/E/L; X403G/K/R; X404K/M; X405Q/S/Y; X416C/Y; X418E/W; X422A; X430A; X440G/H; X441L; X444L/P; and/or X445M/Y, where X is any amino acid corresponding to the equivalent position in SEQ ID NO:4 and/or an equivalent position in a parent glucoamylase or fragment thereof.

33. The glucoamylase variant of claim 32, wherein said one or more amino acid substitutions comprise one or more of S020A/E/P; K021M/S/T/W; E023L/M; E037C; A051K/L/V/Y; G052N; V066A/C/F/M/W; S067A/C; V069C; K073N/P; T077M/P/S; A079R/T; G080H/K; D081N/S; 1084L/V; V092C/M; F094A/G; P102G; S119A/D/G; T121G/L/M/P; E134Q/S; M140C/I/Q; L141F; F156C/P; T157I; N158A/Y; 1164L/T; Y165I/T/W; K166G/F/R; V172G; Y192F; D203C/W; R210L/M/W; D213H/R; N214A/E/Y; S215C/D/F/R; S218A/K/Q/V/Y; S221M/R/T; G222C/M/V; S233M/P/T/Y; W235F/Y; D236A/Q; G238H/S; T243A; V252F; E253K; G274A/D/K; P278A; E281D; F290M/V; F290V; N302H/K/P/Q/S/V/W; N310T/Y; L341M/T; K350C/E/I; N351E/V; T352D/N/S; V354L/M; S370M; S390D/E/L; Q403G/K/R; Y404K/M; H405Q/S/Y; F416C/Y; R418E/W; Y422A; T430A; A440G/H; Q441L; A444L/P; and/or G445M/Y.

34. The glucoamylase variant of any one of claims 1-6, wherein said variant comprises two or more amino acid substitutions comprising a) X236S and X281D; b) X215R and X441W; c) X321D and X434S; d) X143G and X434S; e) X079C and X143G; f) X350T and X434S; g) X351E and X403K; h) X052N and X084L; i) X243P and X290V; j) X290V and X350E; k) X302K and X441W; 1) X156C and X404K; m) X067M and X404K; n) X052N and X141F; o) X052N and X351E; p) X233M and X445Y; q) X066C and X233M; r) X218H and X290V; s) X067M and X302H; t) X066C and X119A; u) X243P and X445Y; v) X192F and X243P; w) X156C and X243P; x) X023L and X066C; y) X023L-X119A; z) X020E and X192F; aa) X192F and X310V; bb) X023M and X302H; cc) X192F and X416Y; dd) X119A and X302H; ee) X235Y and X416Y; ff) X052N and X404K; gg) X023M and X449Y; hh) X158A and X172L; ii) X172L and X290V; jj) X023M and X210L; kk) X210L and X449Y; 11) X157I and X281D; mm) X164T and X215R; nn) X140C and X422V; oo) X119A and X302K; or pp) X052N and X416Y, where X is any amino acid corresponding to the equivalent position in SEQ ID NO:4 and/or an equivalent position in a parent glucoamylase or fragment thereof.

35. The glucoamylase variant of claim 34, wherein said two or more amino acid substitutions comprise a) D236S and E281D; b) S215R and Q441W; c) N321D and A434S; d) A143G and A434S; e) A079C and A143G; f) K350T and A434S; g) N351E and Q403K; h) G052N and I084L; i) T243P and F290V; j) F290V and K350E; k) N302K and Q441W; 1) F156C and Y404K; m) S067M and Y404K; n) G052N and L141F; o) G052N and N351E; p) S233M and G445Y; q) A066C and S233M; r) S218H and F290V; s) S067M and N302H; t) A066C and S119A; u) T243P and G445Y; v) Y192F and T243P; w) F156C and T243P; x) E023L and A066C; y) E023L and S119A; z) S020E and Y192F; aa) Y192F and N310V; bb) E023M and N302H; cc) Y192F and F416Y; dd) S119A and N302H; ee) W235Y and F416Y; ff) G052N and Y404K; gg) E023M and F449Y; hh) N158A and V172L; ii) V172L and F290V; jj) E023M and R210L; kk) R210L and F449Y; 11) T157I and E281D; mm) I164T and S215R; nn) M140C and Y422V; oo) S119A and N302K; or pp) G052N and F416Y.

36. The glucoamylase variant of any one of claims 1-6, wherein said variant comprises three or more amino acid substitutions comprising a) X141F, X281D, and X441W; b) X143G, X321D, and X434S; c) X321D, X350T, and X434S; d) X067M, X158A, and X281D; e) X156C, X192F, and X403K; f) X052N, X140C, and X422V; g) X066C, X119A, and X164T; h) X066C, X233M, and X445Y; i) X156C, X192F, and X243P; j) X023L, X066C, and X119A; k) X023M, X119A, and X404K; 1) X310V, X416Y, and X445Y; m) X158A, X221R, and X290V; n) X023M, X052N, and X404K; o) X081S, X157I, and X236S; p) X243P, X302K, and X416Y; q) X140C, X302K, and X422V; or r) X052N, X416Y, and X445Y, where X is any amino acid corresponding to the equivalent position in SEQ ID NO:4 and/or an equivalent position in a parent glucoamylase or fragment thereof.

37. The glucoamylase variant of claim 36, wherein said three or more amino acid substitutions comprise a) L141F, E281D, and Q441W; b) A143G, N321D, and A434S; c) N321D, K350T, and A434S; d) S067M, N158A, and E281D; e) F156C, Y192F, and Q403K; f) G052N, M140C, and Y422V; g) A066C, S119A, and I164T; h) A066C, S233M, and G445Y; i) F156C, Y192F, and T243P; j) E023L, A066C, and S119A; k) E023M, S119A, and Y404K; 1) N310V, F416Y, and G445Y; m) N158A, S221R, and F290V; n) E023M, G052N, and Y404K; o) D081S, T157I, and D236S; p) T243P, N302K, and F416Y; q) M140C, N302K, and Y422V; or r) G052N, F416Y, and G445Y.

38. The glucoamylase variant of any one of claims 1-6, wherein said variant comprises four or more amino acid substitutions comprising a) X215R, X236S, X281D, and X441W; b) X052N, X084L, X140C, and X422V; c) X020E, X156C, X192F, and X243P; d) X023M, X221R, and X404K; e) X158A, X172L, X221R, and X290V; f) X140C, X165W, X302K, and X422V; or g) X119A, X253F, X310V, and X403K, where X is any amino acid corresponding to the equivalent position in SEQ ID NO:4 and/or an equivalent position in a parent glucoamylase or fragment thereof.

39. The glucoamylase variant of claim 38, wherein said four or more amino acid substitutions comprise a) S215R, D236S, E281D, and Q441W; b) G052N, I084L, M140C, and Y422V; c) S020E, F156C, Y192F, and T243P; d) E023M, S119A, S221R, and Y404K; e) N158A, V172L, S221R, and F290V; f) M140C, Y165W, N302K, and Y422V; or g) S119A, E253F, N310V, and Q403K.

40. The glucoamylase variant of any one of claims 1-6, wherein said variant comprises five or more amino acid substitutions comprising X067M, X157I, X218H, X302H, and X416Y, where X is any amino acid corresponding to the equivalent position in SEQ ID NO:4 and/or an equivalent position in a parent glucoamylase or fragment thereof.

41. The glucoamylase variant of claim 40, wherein said five or more amino acid substitutions comprise S067M, T157I, S218H, N302H, and F416Y.

42. The glucoamylase variant of any one of claims 34-41, wherein said variant glucoamylase exhibits one or more of i) improved hydrolysis of maltose; ii) improved hydrolysis of panose; iii) improved hydrolysis of pullulan; iv) improved hydrolysis of soluble starch; v) improved hydrolysis of maltodextrin; vi) improved thermostability; vii) less reversion to saccharides having a DP greater than or equal to 2; and/or viii) improved saccharification yield of glucose compared to a parent glucoamylase lacking these substitutions.

43. The glucoamylase variant of any one of claims 1-6, wherein said variant comprises substitutions at residue positions corresponding to positions a) 66, 67, and 69; b) 102, 119, and 121; and/or c) 143, 156, 164, 192, and 233 of SEQ ID NO: 3, SEQ ID NO:4 and/or an equivalent position in a parent glucoamylase or fragment thereof.

44. The glucoamylase of claim 43, wherein said variant comprises amino acid substitutions comprising a) X066A/C/F/M/P/T/W, X067A/C/M, and X069A/C/K; b) X102P/G, X119A/D/G, X121G/L/M/P/V; and/or c) X143G, X156C/P, X164L/T, X192F/R, and X233M/PT/Y.

45. The glucoamylase of claim 44, wherein said variant comprises amino acid substitutions comprising a) V066A/C/F/M/P/T/W, S067A/C/M, and V069A/C/K; b) S102P/G, S119A/D/G, T121G/L/M/P/V; and/or c) A143G, F156C/P, 1164L/T, Y192F/R, and S233M/PT/Y.

46. The glucoamylase variant of any one of claims 1-45, further comprising an amino acid substitution at one or more residue positions corresponding to positions 66 and/or 102 of SEQ ID NO:4 and/or an equivalent position in a parent glucoamylase.

47. The glucoamylase variant of claim 46, wherein said one or more amino acid substitutions comprise X066A/C/F/M/P/T/W and/or X102P/G, where X is any amino acid corresponding to the equivalent position in SEQ ID NO:4 and/or an equivalent position in a parent glucoamylase.

48. The glucoamylase variant of claim 47, wherein said one or more amino acid substitutions comprise V066A/C/F/M/P/T/W and/or S102P/G.

49. The glucoamylase variant of any one of claims 1-48, comprising an N-linked glycosylation at position N075 of SEQ ID NO:4 and/or an equivalent position in a parent glucoamylase.

50. The glucoamylase variant of any one of claims 1-49, further comprising one or more amino acid substitutions at residue positions corresponding to positions 81, 83, 153, 370 or 372 of SEQ ID NO:4 and/or an equivalent position in a parent glucoamylase, and wherein said variant glucoamylase exhibits increased glycosylation and increased thermostability compared to a parent glucoamylase lacking one or more of these substitutions.

51. The glucoamylase of claim 50, wherein said one or more substitutions comprise one or more substitutions at position D81N, K83T, A153T, S370N, and/or A372S.

52. A polynucleotide encoding the glucoamylase variant of any one of claims 1-51.

53. A vector comprising the polynucleotide of claim 52.

54. A host cell comprising the polynucleotide of claim 52 or the vector of claim 53.

55. The host cell of claim 54, wherein the host cell is a bacterial or fungal host cell.

56. The host cell of claim 54 or claim 55, wherein the host cell is an Aspergillus spp. a Bacillus spp., or a Trichoderma spp., a Pichia spp., a Myceliophthora spp., or a Saccharomyces spp.

57. An enzyme composition comprising the glucoamylase variant of any one of claims 1-51.

58. The enzyme composition of claim 57, wherein said composition is used in a starch conversion process.

59. The enzyme composition of claim 57, wherein said composition is used in an animal feed formulation.

60. The enzyme composition of claim 57, wherein said composition is used in an alcohol fermentation process.

61. The enzyme composition of claim 57, wherein said composition is used in a process to make a fermented beverage.

62. A method of producing a variant glucoamylase in a host cell comprising a) culturing a host cell transformed with the vector of claim 53 in a culture medium under conditions suitable for production of said glucoamylase variant; and b) producing said variant.

63. The method of claim 62, further comprising recovering the glucoamylase variant from said culture medium.

64. The method of claim 62 or claim 63, wherein the host cell is a bacterial or fungal host cell.

65. The method of any one of claims 62-64, wherein the host cell is an Aspergillus spp. a Bacillus spp., or a Trichoderma spp., a Pichia spp., a Myceliophthora spp., or a Saccharomyces spp.

66. A method of saccharifying a composition comprising starch to produce a composition comprising glucose, wherein said method comprises: a) contacting a starch composition with the glucoamylase variant of any one of claims 1-51; and b) saccharifying the starch composition to produce said glucose composition.

67. The method of claim 67, wherein said composition comprising starch comprises liquefied starch, gelatinized starch, or granular starch.

68. The method of claim 66 or claim 67, wherein the method further comprises c) contacting the starch composition with an alpha-amylase.

69. The method of any one of claims 66-68, wherein the method further comprises d) contacting the starch composition with a pullulanase.

70. The method of any one of claims 66-69, further comprising e) fermenting the glucose composition to produce a fermentation product.

71. The method of claim 70, wherein the fermentation product is an alcohol.

72. The method of claim 71, wherein the alcohol is ethanol or butanol.

73. The method of any one of claims 66-72, further comprising f) adding one or more of an additional glucoamylase, hexokinase, xylanase, glucose isomerase, xylose isomerase, phosphatase, phytase, protease, pullulanase, β-amylase, an additional α-amylase, protease, cellulase, hemicellulase, lipase, cutinase, trehalase, isoamylase, redox enzyme, esterase, transferase, pectinase, alpha-glucosidase, beta-glucosidase, lyase, hydrolase, or a combination thereof, to said starch composition.

74. The method of any one of claims 66-73, wherein the fermentation is a simultaneous saccharification and fermentation (SSF) reaction.
